(19) United States
(12) Patent Application Publication
(10) Pub. No.: US 2018/0362968 A1
(43) Pub. Date: Dec. 20, 2018

(54) METHODS OF LIBRARY CONSTRUCTION FOR TARGET POLYNUCLEOTIDES

(71) Applicant: SomaGenics, Inc., Santa Cruz, CA (US)

(72) Inventors: Sergei A. KAZAKOV, San Jose, CA (US); Sergio BARBERAN-SOLER, Santa Cruz, CA (US); Ryan HOGANS, Santa Cruz, CA (US); Brian H. JOHNSTON, Scotts Valley, CA (US)

(21) Appl. No.: 16/007,769

(22) Filed: Jun. 13, 2018

Related U.S. Application Data
(60) Provisional application No. 62/519,371, filed on Jun. 14, 2017.

(51) Int. Cl. C12N 15/10

(52) U.S. Cl. CPC C12N 15/1068

(57) ABSTRACT

Disclosed herein are methods of constructing a library of target polynucleotides. The present invention finds use in a variety of genomic research and diagnostic applications, including medical, agricultural, food and biodefense fields. Polynucleotides of interest may represent biomarkers of infection (e.g., viral and bacterial), or diseases such as cancer, genetic disorders, and metabolic disorders.

### CROSS-REFERENCE

This application claims the benefit of U.S. Provisional Application No. 62/519,371, filed Jun. 14, 2017, the entire content of which is hereby incorporated by reference.

### GOVERNMENT SUPPORT

This invention was made with government support under Small Business Innovation Research grant 1R43GM115124-01 awarded by the National Institute of Health. The government has certain rights in the invention.

### SEQUENCE LISTING

The instant application contains a Sequence Listing which has been filed electronically in ASCII format and is hereby incorporated by reference in its entirety. Said ASCII copy, created on Jul. 11, 2018, is named 40220-712_201_SL.txt and is 30,265 bytes in size.

### FIELD OF THE INVENTION

The present invention is in the field of molecular and cell biology. More specifically, it concerns methods and compositions that find use in the identification, detection, quantification, expression profiling of small polynucleotides and fragments of large polynucleotides (RNA and DNA) of interest (target polynucleotides), both naturally occurring and synthetic. The present invention finds use in a variety of genomic research and diagnostic applications, including medical, agricultural, food and biodefense fields. Polynucleotides of interest may represent biomarkers of infection (e.g., viral and bacterial), or diseases such as cancer, genetic disorders, and metabolic disorders.

### SUMMARY OF THE INVENTION

Disclosed herein, in some aspects, are methods for detecting a target polynucleotide amongst a plurality of sample polynucleotides in a sample, comprising: ligating a first adapter to a first end of the target polynucleotide via a splint-independent ligation reaction to produce a single-adapter-polynucleotide ligation product (SAP); either: ligating a second adapter to a second end of the SAP to produce a double-adapter-polynucleotide ligation product (DAP); and optionally hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; or circularizing the SAP by intramolecular ligation of the SAP ends to produce a circular single adapter-polynucleotide ligation product (CSAP); and optionally hybridizing the primer to the CSAP and extending by the polymerase to produce the primer extension product comprising the sequence complementary to the target polynucleotide; and optionally amplifying the primer extension product to produce the amplified primer extension product comprising sequence(s) corresponding and/or complementary to the target polynucleotide; hybridizing a target-specific oligonucleotide probe (TSP) to at least a portion of the DAP, CSAP, primer extension product, or amplified primer extension product to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and optionally amplifying the released product to produce an amplified released product; and detecting the released product or amplified released product, wherein the amount of the released product or amplified released product correlates with the amount of the target polynucleotide. In some instances, the target polynucleotide is DNA and the released product or amplified released product comprises a sequence that corresponds to a sequence of the target polynucleotide. In some instances, the target polynucleotide is DNA and the released product or amplified released product comprises a sequence that is complementary to a sequence of the target polynucleotide. In some instances, the target polynucleotide is RNA and the released product or amplified released product comprises a sequence that corresponds to a sequence of the target polynucleotide. In some instances, the target polynucleotide is RNA and the released product or amplified released product comprises a sequence that is complementary to a sequence of the target polynucleotide. In some instances, methods comprise ligating the second adapter to the second end of the SAP to produce the DAP. In some instances, hybridizing the TSP occurs before ligating of the second adapter. In some instances, hybridizing the TSP occurs directly before ligating of the second adapter. In some instances, ligating of the second adapter occurs before hybridizing the TSP. In some instances, ligating of the second adapter occurs directly before hybridizing the TSP. In some instances, hybridizing the TSP occurs before circularizing. In some instances, hybridizing the TSP occurs directly before circularizing. In some instances, circularizing occurs before hybridizing the TSP. In some instances, circularizing occurs directly before hybridizing the TSP. In some instances, methods comprise hybridizing a first TSP before circularizing and hybridizing a second TSP after circularizing. In some instances, hybridizing the TSP comprises hybridizing of one TSP oligonucleotide for each product produced in step (a) and/or (b). In some instances, hybridizing of the TSP comprises hybridizing two or more TSP oligonucleotides to the same product produced in step (a) and/or (b). In some instances, methods comprise ligating the second adapter in step (i) and/or circularizing in step (ii) via a splint-independent reaction. In some instances, methods comprise ligating the second adapter in step (i) and/or circularizing in step (ii) via splint-dependent reaction, wherein the TSP oligonucleotide serves as a splint. In some instances, amplifying does not occur in step (b). In some instances, methods comprise amplifying the released product. In some instances, methods comprise sequencing the released product. In some instances, detecting comprises performing a microarray detection of the released product. In some instances, detecting comprises performing RT-qPCR, qPCR, PCR arrays or digital PCR on the released product. In some instances, detecting comprises detecting a plurality of target polynucleotides in the sample. In some instances, detecting comprises detecting a plurality of target polynucleotides in the sample simultaneously. In some instances, the TSP is at least partially complementary to the target polynucleotide. In some instances, the TSP is at least partially complementary to the first adapter or second adapter. In some instances, said first adapter is ligated to the 5′ end of the target polynucleotide. In some instances, said first adapter is ligated to the 3′ end of the target polynucleotide. In some instances, said adapter comprises a 5′-proximal segment and a 3′-proximal segment, and wherein at least one of the 5′ proximal segment or the 3′ proximal segment comprises a sequencing adapter. In some instances, hybridizing with the TSP occurs in solution followed by capture of the hybridized TSP on a solid support in a later step or steps. In some instances, hybridizing with the TSP occurs on a solid support. In some instances, said TSP hybridizes only to target polynucleotide-specific sequences. In some instances, said TSP hybridizes to at least a portion of both target polynucleotide and at least a portion of the first or second adapter of the SAP. In some instances, the TSP hybridizes to at least one adapter-polynucleotide ligation product that is 50 or fewer nucleotides or base pairs in length. In some instances, detecting comprises hybridizing a first primer comprising a sequence at least partially complementary to the 5′-proximal segment of said first adapter or second adapter. In some instances, detecting comprises hybridizing a first primer comprising a sequence at least partially complementary to the 3′-proximal segment of said first adapter or second adapter. In some instances, methods comprise extending the primer with the polymerase to produce a plurality of primer extension products, wherein each of said primer extension products is complementary to at least a portion of one target polynucleotide of the sample, and wherein the primer extension products are flanked by at least a portion of a sequence corresponding to or complementary to the sequencing adapter. In some instances, methods comprise amplifying said plurality of primer extension products using a second primer and a third primer, wherein the sequence of the third primer is at least partially complementary to the 3′-proximal segment of said adapter, to produce amplicon(s). In some instances, methods comprise using the amplicons as a sequencing library. In some instances, methods comprise amplifying said plurality of primer extension products using a second primer and a third primer, wherein the sequence of the third primer is at least partially complementary to the 5′-proximal segment of said adapter, to produce amplicon(s) comprising the sequencing library. In some instances, the first primer and the second primer have the same sequence. In some instances, methods comprise hybridizing and extending at least one primer to the first or second adapter of the DAP or CSAP by a polymerase to produce a complementary DNA (cDNA) or other primer extension product. In some instances, the target polynucleotide comprises naturally occurring RNA and synthetic RNA. In some instances, the target polynucleotide comprises circular RNA. In some instances, the target polynucleotide comprises single-stranded RNA. In some instances, the target polynucleotide comprises double-stranded RNA. In some instances, the target polynucleotide comprises naturally occurring DNA and synthetic DNA. In some instances, the target polynucleotide comprises circular DNA. In some instances, the target polynucleotide comprises single-stranded DNA. In some instances, target polynucleotide comprises double-stranded DNA. In some instances, methods comprise ligating a first adapter to a first end of the target polynucleotide via a splint-independent ligation reaction to produce a single-adapter-polynucleotide ligation product (SAP); circularizing the SAP by intramolecular ligation of the SAP ends to produce a circular single adapter-polynucleotide ligation product (CSAP); and hybridizing the primer to the CSAP and extending by the polymerase to produce the primer extension product comprising the sequence complementary to the target polynucleotide; hybridizing a target-specific oligonucleotide probe (TSP) to at least a portion of the primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and amplifying the released product to produce an amplified released product; and detecting the amplified released product, wherein the amount of the released product or amplified released product correlates with the amount of the target polynucleotide. In some instances, methods comprise ligating a first adapter to a first end of the target polynucleotide via a splint-independent ligation reaction to produce a single-adapter-polynucleotide ligation product (SAP); circularizing the SAP by intramolecular ligation of the SAP ends to produce a circular single adapter-polynucleotide ligation product (CSAP); and hybridizing a target-specific oligonucleotide probe (TSP) to at least a portion of the CSAP to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product and amplifying the released product to produce an amplified product wherein the amplifying comprises hybridizing the primer to the released product and extending by the polymerase to produce the primer extension product comprising the sequence complementary to the target polynucleotide; and detecting the amplified product, wherein the amount of the amplified product correlates with the amount of the target polynucleotide. In some instances, methods comprise ligating a first adapter to a first end of the target polynucleotide via a splint-independent ligation reaction to produce a single-adapter-polynucleotide ligation product (SAP); hybridizing a target-specific oligonucleotide probe (TSP) to at least a portion of the SAP to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and optionally amplifying the released product to produce an amplified released product; and either: ligating a second adapter to a second end of the SAP to produce a double-adapter-polynucleotide ligation product (DAP); and optionally hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; or circularizing the SAP by intramolecular ligation of the SAP ends to produce a circular single adapter-polynucleotide ligation product (CSAP); and optionally hybridizing the primer to the CSAP and extending by the polymerase to produce the primer extension product comprising the sequence complementary to the target polynucleotide; and optionally amplifying the primer extension product to produce the amplified primer extension product comprising sequence(s) corresponding and/or complementary to the target polynucleotide; detecting the released product or amplified released product, wherein the amount of the released product or amplified released product correlates with the amount of the target polynucleotide. In some instances, methods comprise ligating a first (or single) adapter to a first end of the target polynucleotide via a splint-independent ligation reaction to produce a single-adapter-polynucleotide ligation product (SAP); hybridizing a target-specific oligonucleotide probe (TSP) to at least a portion of the SAP to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; circularizing the SAP by intramolecular ligation of the SAP ends to produce a circular single adapter-polynucleotide ligation product (CSAP); and hybridizing the primer to the CSAP and extending by the polymerase to produce the primer extension product comprising the sequence complementary to the target polynucleotide; and amplifying the primer extension product to produce the amplified primer extension product comprising sequence(s) corresponding and/or complementary to the target polynucleotide; and detecting the released product or amplified released product, wherein the amount of the released product or amplified released product correlates with the amount of the target polynucleotide. In some instances, methods comprise ligating a first adapter to a first end of the target polynucleotide via a splint-independent ligation reaction to produce a single-adapter-polynucleotide ligation product (SAP); either: ligating a second adapter to a second end of the SAP to produce a double-adapter-polynucleotide ligation product (DAP); or circularizing the SAP by intramolecular ligation of the SAP ends to produce a circular single adapter-polynucleotide ligation product (CSAP); hybridizing a target-specific oligonucleotide probe (TSP) to at least a portion of the DAP or CSAP to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and hybridizing a primer to the released product and extending by the polymerase to produce the primer extension product comprising the sequence complementary to the target polynucleotide; and optionally amplifying the primer extension product to produce an amplified released product; and detecting the primer extension product or amplified released product, wherein the amount of the primer extension product or amplified released product correlates with the amount of the target polynucleotide.

### BRIEF DESCRIPTION OF THE DRAWINGS

FIG. 1A-FIG. 1E. Schematic representations of target-specific oligonucleotide probes (TSPs) comprising a group that allows their non-covalent or covalent attachment (or immobilization) to a solid support. FIGS. 1 A-1C: Examples of TSPs carrying a hapten group (Z) such as biotin or digoxigenin attached to one of the TSP ends or internally via non-nucleotide and/or oligonucleotide linkers that can bind with high affinity to surface-bound hapten-specific proteins such as streptavidin or a hapten-specific antibody. FIGS. 1 D-1E: Examples of TSPs extended at one end by an oligonucleotide segment that is complementary to a capture oligonucleotide probe attached to a solid support such as magnetic beads.

FIG. 2. Schematic representations of isolation of target polynucleotides from a pool of sample polynucleotides using TSPs. Single-stranded (or denatured double-stranded) RNA and/or DNA polynucleotides are hybridized with TSPs that are specific to target polynucleotides. The number of target polynucleotides (and target-specific probes) may vary from one to several thousand. Capture of TSP-polynucleotide hybrids on a solid support (e.g., magnetic beads) allows concentrating the target polynucleotides from diluted samples and/or washing off non-target polynucleotides and other solutes, including inhibitors of certain enzymatic reactions that may be present in samples. The concentrated and purified target polynucleotides are then released into solution for processing, such as ligation of adapter(s) and circularization, and analysis.

FIG. 3A-FIG. 3B. Schemes to exemplify the sequential ligation of 3′-adapter and 5′-adapter to the ends of sample polynucleotides and capture of target polynucleotide-adapter ligation products. FIG. 3A: Capture of target polynucleotides ligated to 3′-adapter and separation of the ligation product from the unligated adapter to avoid the formation of adapter dimers in the subsequent adapter ligation step. FIG. 3B: Capture of target polynucleotides ligated to both 3′-adapter and 5′-adapter, and separation of the ligation products from the unligated adapter(s) as well as adapter dimers.

FIG. 4A-FIG. 4D. Schemes to exemplify the sequential ligation of a 5′-adapter and a 3′-adapter to the ends of sample polynucleotides and capture of these polynucleotide-adapter ligation products. For each scheme, following capture of target polynucleotides ligated to a 5′-adapter or to a 5′-adapter and a 3′-adapter, they are separated from non-target polynucleotides, adapter dimers and unligated adapters. The captured products are then released into solution. FIG. 4A: A scheme demonstrating splint-independent ligation of a 5′-adapter to the 5′ end of a target polynucleotide. FIG. 4B: A scheme demonstrating splint-independent ligation of a 3′-adapter to the 3′-end of target polynucleotides ligated to 5′-adapter. FIG. 4C: A scheme demonstrating splint-dependent ligation of a 3′-adapter to the 3′-end of target polynucleotides ligated to a 5′-adapter, wherein said splint comprises a TSP that is complementary to a 3′-end proximal segment of the target polynucleotide and to a 5′-end proximal segment of the 3′-adapter, thereby aligning these ends head-to-tail within the duplex formed with the splint. FIG. 4D: A scheme comprising splint-dependent ligation of a 3′-adapter to the 3′-end of target polynucleotides ligated to 5′-adapter, wherein said splint comprises a TSP that comprises: (i) a 3′-end proximal segment, which is complementary to a 3′-end segment of the target polynucleotide; (ii) a 5′-end proximal segment, which is complementary to a 5′-end proximal segment of the 3′ adapter; and (iii) a linker connecting the 3′-end proximal segment and the 5′-end proximal segment of the TSP, wherein the linker is not complementary to one or more nucleotide(s) at the 3′ end of the polynucleotide and wherein the linker is not complementary to one or more of the nucleotide(s) at the 3′ end of the polynucleotide.

FIG. 5A-FIG. 5B. Schemes to exemplify the capture of target-specific cDNAs (complementary DNAs) after reverse transcription of polynucleotide-adapter ligation products. In the schemes shown, both polynucleotides and 5′-adapter comprise RNA nucleotides while the 3′-adapter comprises either DNA (FIG. 5A) or RNA nucleotides (FIG. 5B). After reverse transcription and degradation of RNA templates (e.g., by RNase H), the cDNAs comprising antisense sequences of target polynucleotides are captured and separated from cDNA products from non-target polynucleotides and adapter dimers.

FIG. 6. Schemes to exemplify the capture of target-specific cDNAs after RT-PCR or PCR amplification of polynucleotide-adapter ligation products. The reverse transcription and optional degradation of RNA templates may only be required if target polynucleotides and/or one or both adapters comprise RNA nucleotides. PCR amplification of polynucleotides ligated with two (5′- and 3′-) adapters in the presence of an excess of one of the primers generates single-stranded amplicons that are captured and separated from the amplification products related to non-target polynucleotides and adapter dimers.

FIG. 7. Schemes to exemplify the preparation of strand-specific sequencing libraries from cDNAs comprising sequences of 5′-adapter, target polynucleotides and 3′-adapter. The adapters comprise sequences that are compatible with PCR primers specific for the NGS method used for sequencing.

FIG. 8A-FIG. 8B. Schemes to exemplify the ligation of a single combo adapter (CAD) to the ends of sample polynucleotides and capture of target polynucleotide-CAD ligation products. The CAD comprises sequences of the 3′-adapter and 5′-adapter presented in FIGS. 3-7, but in opposite order from that of the adapter dimer (compare with FIG. 4B). Optionally, these 3′- and 5′-adapter sequences within the CAD can be separated by one or more template-deficient modifications that stop primer extension by a polymerase. The CAD can be ligated either to the 3′-end (FIG. 8A) or 5′-end (FIG. 8B) of the polynucleotide to form polynucleotide-CAD ligation products (PCADs). Different combinations of terminal groups at the polynucleotide and CAD ends allow different enzymatic ligation steps. Some terminal groups also can serve as reversible blocking groups to prevent circularization (and multimerization) of the polynucleotide and/or CAD that may compete with ligation of polynucleotide with CAD. Capture of target polynucleotides ligated to the CAD allows separation of the PCADs from the unligated CAD.

FIG. 9A-FIG. 9C. Schemes to exemplify the circularization of polynucleotide-CAD ligation products and capture of circularized target polynucleotide-CAD ligation products. FIG. 9A: Splint-independent circularization of the polynucleotide-CAD ligation products (PCADs) and unligated CAD creates templates with the same order of 5′- and 3′-adapters relative to polynucleotide insert as the two-adapter ligation approach (see FIG. 3B). To allow the circularization of the PCADs, the reversible blocking groups at the available ends of polynucleotide and CAD segments should be repaired (e.g., by phosphorylation or de-phosphorylation). Such repair also may allow circularization and multimerization of CADs that may be present in access relative to polynucleotide-CAD ligation products. To prevent the circularization of unligated CAD, the CAD end that participates in ligation to the polynucleotide can be enzymatically or chemically blocked. FIG. 9B: A scheme depicting splint-dependent circularization of 3′-adapter, wherein a TSP serving as a splint (or template) is complementary to a 3′-end proximal segment of the target polynucleotide and to a 5′-end proximal segment of the 3′-adapter, thereby aligning these ends head-to-tail within the duplex formed with the splint. FIG. 9C: The circularized polynucleotide-CAD ligation products can be captured and purified from circular non-target polynucleotide-CAD ligation products and circular CAD similar to their linear counterparts (see, e.g., FIG. 3B).

FIG. 10A-FIG. 10B. Schemes to exemplify the capture of target-specific cDNAs after reverse transcription of the circular polynucleotide-combo adapter ligation products (PCADs). In the schemes shown, both polynucleotides and 5′-adapter comprise RNA nucleotides while the 3′-adapter comprises either DNA (FIG. 10A) or RNA nucleotides (FIG. 10B). Unrestricted primer extension on the circular PCAD template can result in synthesis by rolling-circle amplification (RCA) of multimeric cDNAs comprising multiple repeats of the adapter and polynucleotide sequences. Alternatively (as shown in these figures), the PCAD may comprise a CAD with template-deficient modification(s) as described in FIG. 8. In the latter case, primer extension on the circular PCAD template stops at the template-deficient modification(s) after one round, thus preventing RCA. This product of primer extension (cDNA) comprises sequences complementary to the PCAD and contains sequences of a single polynucleotide inserted between the sequencing adapters exactly in the same order as they appear in conventional methods of sequencing library preparation using ligation of two separate adapters to each polynucleotide (see, e.g., FIG. 5). After reverse transcription and degradation of RNA templates (e.g., by RNase H), the cDNAs comprising antisense sequences of target polynucleotides are captured and separated from cDNA products from non-target polynucleotides and adapter dimers similar to what is shown in FIG. 5. By limiting the method to a single round of primer extension, the methods disclosed herein provide several advantages. One advantage is the generation of standard-length PCR amplicons directly compatible with next generation sequencing (see, e.g., FIG. 7). Another advantage is reduced sequencing bias for sample polynucleotides varying in sequence and length since these various polynucleotides can be amplified by RCA with different efficiency.

FIG. 11A-FIG. 11B. Schemes to exemplify the preparation of target miRNA sequencing libraries (as described, e.g., in Example 1). After ligation of the combo adapter (CAD) to miRNAs, target-specific oligonucleotides are used to capture the target miRNA-CAD ligation products and purify them from unligated CAD and non-target miRNA-CAD ligation products as described, e.g., in FIG. 8A. The purified target miRNA-CAD ligation products are then released into solution, circularized and RT-PCR-amplified to generate a sequencing library that is free from CAD and non-target miRNA amplicons.

FIG. 12. Targeted sequencing of selected miRNAs in plasma samples. The upper panel shows results from a standard non-targeted sequencing approach that profiles all cell-free miRNAs isolated from the plasma samples. The lower panel shows results of miRNA sequencing from the same plasma samples using a targeted-sequencing approach for eight selected miRNAs (see Example 11).

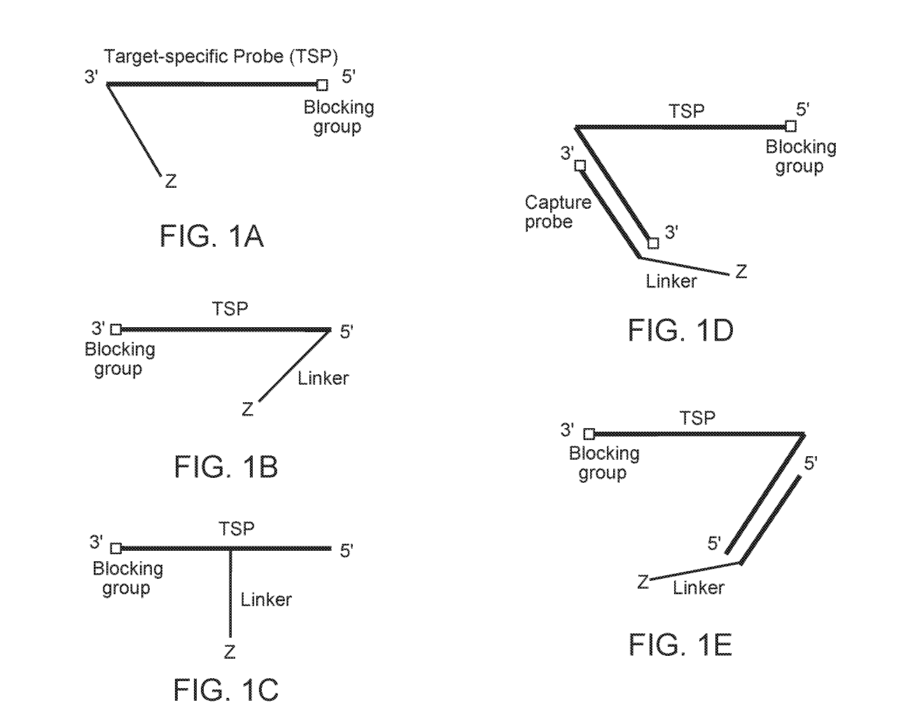

### DETAILED DESCRIPTION OF THE INVENTION

The decreasing cost of sequencing has made it an attractive and powerful tool for quantifying levels of polynucleotides, particularly RNA, in biological samples. However, when species of interest are present at lower abundance, sequencing must be done at greater depth, which is costly, because most of the reads generated derive from the more abundant species. Targeted sequencing overcomes this problem but there is a lack of convenient, accurate methods of targeted sequencing for small RNA (under 250 nucleotides). Hybridization of a TSP with target polynucleotides and/or one or more product(s) comprising sequences specific to target polynucleotides and capture of the hybridization products on a solid support allows concentration of these nucleic acid species from dilute samples and/or washing away of unrelated polynucleotides and other solutes, including inhibitors of certain enzymatic reactions that may be present in samples. Examples of unrelated polynucleotides include ribosomal RNA, tRNA and their fragments, and/or overexpressed non-coding RNAs. Also, target-specific capture of sense or antisense strands of DNA or double-stranded RNAs (e.g., viral RNA) would allow strand specific detection of target polynucleotides. The concentrated and purified nucleic acids comprising sequences specific to target polynucleotides are then released into solution for further procedures such as ligation of adapter(s), circularization, hybridization with primers, primer extension, amplification and detection.

The main problem in detecting target polynucleotides using a hybridization with a TSP is the low fidelity (or sequence-specificity) of hybridization, especially under conditions where efficiency of hybridization is maximized for higher sensitivity. For microarrays or other hybridization-based assay that rely on hybridization to simultaneously capture and detect target sequences highly-specific hybridization is essential. In contrast, methods disclose herein, which are useful for detection of target polynucleotides, require neither highly-specific nor highly efficient hybridization for either hybridization step.

In contrast to conventional targeted sequencing approaches that use target-specific probes to capture, release and then sequence long target polynucleotides by standard methods, methods disclosed herein comprise preparing sequencing libraries of target polynucleotides that comprise capture and purification of one or more of the products of target polynucleotide processing (such as SAP, DAP, CSAP, their primer extension products, and/or products of their amplification). These methods are based on the unexpected result that more than one hybridization step is usually required for optimal detection of the target polynucleotides, because a single hybridization step never allows 100% capture and purification of the target polynucleotides or products of target polynucleotide processing.

Methods disclosed herein are especially useful for highly multiplexed analysis of multiple target polynucleotides and for analysis of samples with ultra-low levels of target polynucleotides such as single cells or cell-free biofluid samples. The present methods allow detection of multiple target polynucleotides at levels that could not be reliably detected by other methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

### Certain Terminologies

Unless defined otherwise, all technical and scientific terms used herein have the same meaning as is commonly understood by one of skill in the art to which the claimed subject matter belongs. It is to be understood that the foregoing general description and the following examples are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. For example, “about 5 μL” means “about 5 μL” and also “5 μL.” Generally, the term “about” includes an amount that would be expected to be within experimental error. The term “about” includes values that are within 10% less to 10% greater of the value provided. For example, “about 50%” means “between 45% and 55%.” Also, by way of example, “about 30” means “between 27 and 33.”

The section headings used herein are for organizational purposes only and are not to be construed as limiting the subject matter described.

The terms “5′-proximal segment” and “3′-proximal segment” refer to independent parts of the combo adapters disclosed herein, wherein the 5′-proximal segment comprises the 5′-end of the combo adapter and the 3′-proximal segment comprises the 3′-end of the combo adapter, respectively, and wherein the 5′-proximal and 3′-proximal segments are linked to each other either by at least one nucleotide, internucleotide bond or non-nucleotide linker. The 5′ proximal segment or the 3′ proximal segment may be about one to about a hundred nucleotides long. In some embodiments, the 5′ proximal segment or the 3′ proximal segment are about 5 to about 70 nucleotides long. In some embodiments, the 5′ proximal segment or the 3′ proximal segment are about 15 to about 40 nucleotides long. In some embodiments, the 5′ proximal segment or the 3′ proximal segment are about 20 to about 27 nucleotides long. In some embodiments, the 5′ proximal segment and the 3′ proximal segment are of about the same length. In some embodiments, the 5′ proximal segment and the 3′ proximal segment are of the same length. In some embodiments, the 5′ proximal segment and the 3′ proximal segment are different lengths. In some embodiments, the 5′ proximal segment or the 3′ proximal segment consist of one nucleotide to 100 nucleotides. In some embodiments, the 5′ proximal segment or the 3′ proximal segment consist of 5 to 70 nucleotides. In some embodiments, the 5′ proximal segment or the 3′ proximal segment consist of 15 to 40 nucleotides. In some embodiments, the 5′ proximal segment or the 3′ proximal segment consist of 20 to 27 nucleotides.

The term “sequencing adapter” refers to nucleotide sequences which have to be added to one or both ends of a sample polynucleotide or its fragment in order for the sample polynucleotide or its fragment to be sequenced. Sequencing can occur either directly (without amplification) or after amplification using extended (combo) primers wherein either the sequencing adapter or extended primers comprise a primer binding site, a capture oligonucleotide binding site, a polymerase binding site, a sequencing bar-code, an indexing sequence, at least one random nucleotide, a unique molecular identifier (UMI), sequencing flow-cell binding sites, and combinations thereof.

The term “combo primer” refers to a primer comprising at its 3′ end a sequence [that is] specific (complementary or corresponding) to the 5′- or 3′-proximal segment of the CAD and has a 5′-end extension accommodating one or more additional sequences (e.g., sequencing index, bar-code, randomized sequence, unique molecular identifier (UMI), sequencing primer binding site or flow-cell binding site, or a combination thereof). The term “combo primer” may also be referred to herein as a “combo PCR primer,” “combo reverse primer,” “combo forward primer,” and an “extended (combo) primer.”

The term “detection sequences” refers to nucleotide sequences that allow a sample polynucleotide or its fragment to be detected either directly or after amplification, using detection techniques known in the art.

The terms “5′-end” and “3′-end” of a nucleic acid are standard terms of molecular biology known in the art, wherein these terms refer to the 5′ and 3′ carbons on the sugar terminal residues.

The terms “splint-dependent ligation” and “template-dependent ligation” may be used interchangeably herein and refer to ligation of the ends of “donor” and acceptor nucleic acid(s) that are brought to proximity by hybridization to the same splint or template nucleic acid. Such ligation reactions require complete or partial complementarity between the splint (or template) nucleic acid to both “donor” and acceptor nucleic acid(s).

The term non-nucleotide residue refers to a residue that is not chemically classified as nucleic acid residue. The non-nucleotide residue may be synthetically inserted (serve as a linker or a spacer) between nucleic acid residues or be attached to nucleic acid ends (terminal groups). Examples of non-nucleotide residues include (but are not limited to): disulfide (S—S), 3′ Thiol Modifier C3 S—S, a propanediol (C3 Spacer), a hexanediol (six carbon glycol spacer), a triethylene glycol (Spacer 9) and hexaethylene glycol (Spacer 18).

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

Disclosed herein, in some aspects, are methods for detecting a target polynucleotide amongst a plurality of sample polynucleotides in a sample, comprising: ligating a first adapter to a first end of the target polynucleotide via a splint-independent ligation reaction to produce a single-adapter-polynucleotide ligation product (SAP); either: ligating a second adapter to a second end of the SAP to produce a double-adapter-polynucleotide ligation product (DAP); and optionally hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; or circularizing the SAP by intramolecular ligation of the SAP ends to produce a circular single adapter-polynucleotide ligation product (CSAP); and optionally hybridizing the primer to the CSAP and extending by the polymerase to produce the primer extension product comprising the sequence complementary to the target polynucleotide; and optionally amplifying the primer extension product to produce the amplified primer extension product comprising sequence(s) corresponding and/or complementary to the target polynucleotide; hybridizing a target-specific oligonucleotide probe (TSP) to at least a portion of the DAP, CSAP, primer extension product, or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and optionally amplifying the released product to produce an amplified released product; and detecting the released product or amplified released product, wherein the amount of the released product or amplified released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and optionally hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; hybridizing a TSP to at least a portion of the DAP, primer extension product, or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and optionally amplifying the released product to produce an amplified released product; and detecting the released product or amplified released product, wherein the amount of the released product or amplified released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; hybridizing a TSP to at least a portion of the DAP, primer extension product, or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and optionally amplifying the released product to produce an amplified released product; and detecting the released product or amplified released product, wherein the amount of the released product or amplified released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and hybridizing a TSP to at least a portion of the DAP to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and optionally amplifying the released product to produce an amplified released product; and detecting the released product or amplified released product, wherein the amount of the released product or amplified released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and hybridizing a primer to the DAP and extending by a polymerase to produce a primer extension product comprising a sequence complementary to the target polynucleotide; and amplifying the primer extension product to produce an amplified primer extension product comprising sequence(s) corresponding and/or complementary to the target polynucleotide; hybridizing a TSP to at least a portion of the amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and optionally amplifying the released product to produce an amplified released product; and detecting the released product or amplified released product, wherein the amount of the released product or amplified released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and optionally hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; hybridizing a TSP to at least a portion of the DAP, primer extension product, or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and amplifying the released product to produce an amplified released product; and detecting the amplified released product, wherein the amount of the amplified released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and optionally hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; hybridizing a TSP to at least a portion of the DAP, primer extension product, or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and detecting the released product, wherein the amount of the released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; hybridizing a TSP to at least a portion of the DAP, primer extension product, or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and optionally amplifying the released product to produce an amplified released product; and detecting the released product or amplified released product, wherein the amount of the released product or amplified released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; hybridizing a TSP to at least a portion of the DAP, primer extension product, or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and amplifying the released product to produce an amplified released product; and detecting the amplified released product, wherein the amount of the amplified released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and hybridizing a TSP to at least a portion of the DAP, to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and detecting the released product, wherein the amount of the released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and hybridizing a primer to the DAP and extending by a polymerase to produce a primer extension product comprising a sequence complementary to the target polynucleotide; and amplifying the primer extension product to produce an amplified primer extension product comprising sequence(s) corresponding and/or complementary to the target polynucleotide; hybridizing a TSP to at least a portion of the amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and amplifying the released product to produce an amplified released product; and detecting the amplified released product, wherein the amount of the amplified released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and hybridizing a primer to the DAP and extending by a polymerase to produce a primer extension product comprising a sequence complementary to the target polynucleotide; and amplifying the primer extension product to produce an amplified primer extension product comprising sequence(s) corresponding and/or complementary to the target polynucleotide; hybridizing a TSP to at least a portion of the amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and detecting the released product, wherein the amount of the released product correlates with the amount of the target polynucleotide.

In some instances, methods disclosed herein comprise ligating a first adapter to a first end of the target polynucleotide via a splint-independent ligation reaction to produce a SAP; circularizing the SAP by intramolecular ligation of the SAP ends to produce a CSAP; and optionally hybridizing the primer to the CSAP and extending by the polymerase to produce the primer extension product comprising the sequence complementary to the target polynucleotide; and optionally amplifying the primer extension product to produce the amplified primer extension product comprising sequence(s) corresponding and/or complementary to the target polynucleotide; hybridizing a TSP to at least a portion of the CSAP, primer extension product, or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and detecting the released product, wherein the amount of the released product correlates with the amount of the target polynucleotide.

In some instances, methods disclosed herein comprise ligating a first adapter to a first end of the target polynucleotide via a splint-independent ligation reaction to produce a SAP; circularizing the SAP by intramolecular ligation of the SAP ends to produce a CSAP; hybridizing the primer to the CSAP and extending by the polymerase to produce the primer extension product comprising the sequence complementary to the target polynucleotide; optionally amplifying the primer extension product to produce the amplified primer extension product comprising sequence(s) corresponding and/or complementary to the target polynucleotide; hybridizing a TSP to at least a portion of the primer extension product, or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and detecting the released product, wherein the amount of the released product correlates with the amount of the target polynucleotide.

In some instances, methods disclosed herein comprise ligating a first adapter to a first end of the target polynucleotide via a splint-independent ligation reaction to produce a SAP; circularizing the SAP by intramolecular ligation of the SAP ends to produce a CSAP; and hybridizing the primer to the CSAP and extending by the polymerase to produce the primer extension product comprising the sequence complementary to the target polynucleotide; and amplifying the primer extension product to produce the amplified primer extension product comprising sequence(s) corresponding and/or complementary to the target polynucleotide; hybridizing a TSP to at least a portion of the amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and detecting the released product, wherein the amount of the released product correlates with the amount of the target polynucleotide.

In some instances, methods disclosed herein comprise ligating a first adapter to a first end of the target polynucleotide via a splint-independent ligation reaction to produce a SAP; circularizing the SAP by intramolecular ligation of the SAP ends to produce a CSAP; and optionally hybridizing the primer to the CSAP and extending by the polymerase to produce the primer extension product comprising the sequence complementary to the target polynucleotide; hybridizing a TSP to at least a portion of the C SAP, primer extension product, produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and detecting the released product, wherein the amount of the released product correlates with the amount of the target polynucleotide.

In some instances, methods disclosed herein comprise ligating a first adapter to a first end of the target polynucleotide via a splint-independent ligation reaction to produce a SAP; circularizing the SAP by intramolecular ligation of the SAP ends to produce a CSAP; hybridizing a TSP to at least a portion of the CSAP produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and detecting the released product, wherein the amount of the released product correlates with the amount of the target polynucleotide.

In some instances, methods disclosed herein comprise ligating a first adapter to a first end of the target polynucleotide via a splint-independent ligation reaction to produce a SAP; circularizing the SAP by intramolecular ligation of the SAP ends to produce a CSAP; and optionally hybridizing the primer to the CSAP and extending by the polymerase to produce the primer extension product comprising the sequence complementary to the target polynucleotide; and optionally amplifying the primer extension product to produce the amplified primer extension product comprising sequence(s) corresponding and/or complementary to the target polynucleotide; hybridizing a TSP to at least a portion of the CSAP, primer extension product, or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and amplifying the released product to produce an amplified released product; and detecting the amplified released product, wherein the amount of the amplified released product correlates with the amount of the target polynucleotide.

In some instances, methods disclosed herein comprise ligating a first adapter to a first end of the target polynucleotide via a splint-independent ligation reaction to produce a SAP; circularizing the SAP by intramolecular ligation of the SAP ends to produce a CSAP; hybridizing the primer to the CSAP and extending by the polymerase to produce the primer extension product comprising the sequence complementary to the target polynucleotide; optionally amplifying the primer extension product to produce the amplified primer extension product comprising sequence(s) corresponding and/or complementary to the target polynucleotide; hybridizing a TSP to at least a portion of the primer extension product, or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and amplifying the released product to produce an amplified released product; and detecting the amplified released product, wherein the amount of the amplified released product correlates with the amount of the target polynucleotide.

In some instances, methods disclosed herein comprise ligating a first adapter to a first end of the target polynucleotide via a splint-independent ligation reaction to produce a SAP; circularizing the SAP by intramolecular ligation of the SAP ends to produce a CSAP; and hybridizing the primer to the CSAP and extending by the polymerase to produce the primer extension product comprising the sequence complementary to the target polynucleotide; and amplifying the primer extension product to produce the amplified primer extension product comprising sequence(s) corresponding and/or complementary to the target polynucleotide; hybridizing a TSP to at least a portion of the amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and amplifying the released product to produce an amplified released product; and detecting the amplified released product, wherein the amount of the amplified released product correlates with the amount of the target polynucleotide.

In some instances, methods disclosed herein comprise ligating a first adapter to a first end of the target polynucleotide via a splint-independent ligation reaction to produce a SAP; circularizing the SAP by intramolecular ligation of the SAP ends to produce a CSAP; and optionally hybridizing the primer to the CSAP and extending by the polymerase to produce the primer extension product comprising the sequence complementary to the target polynucleotide; hybridizing a TSP to at least a portion of the CSAP, primer extension product, produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and amplifying the released product to produce an amplified released product; and detecting the amplified released product, wherein the amount of the amplified released product correlates with the amount of the target polynucleotide.

In some instances, methods disclosed herein comprise ligating a first adapter to a first end of the target polynucleotide via a splint-independent ligation reaction to produce a SAP; circularizing the SAP by intramolecular ligation of the SAP ends to produce a CSAP; hybridizing a TSP to at least a portion of the CSAP produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and amplifying the released product to produce an amplified released product; and detecting the amplified released product, wherein the amount of the amplified released product correlates with the amount of the target polynucleotide.

In some instances, methods disclosed herein comprise ligating a first adapter to a first end of the target polynucleotide via a splint-independent ligation reaction to produce a SAP; ligating a second adapter to a second end of the SAP to produce a DAP; and hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; hybridizing a TSP to at least a portion of the primer extension product or amplified primer extension product produced to produce a TSP-hybridized product, and capturing the TSP-hybridized product on a solid support to produce a captured TSP-hybridized product; removing a component from the sample that is not captured on the solid support; releasing the captured TSP-hybridized product into solution to produce a released product; and optionally amplifying the released product to produce an amplified released product; and detecting the released product or amplified released product, wherein the amount of the released product or amplified released product correlates with the amount of the target polynucleotide.

Provided herein are methods for detecting a target polynucleotide amongst a plurality of sample polynucleotides in a sample. In some embodiments, the methods comprise ligating a first adapter to a first end of a polynucleotide to produce a single-adapter-polynucleotide ligation product (SAP). In some embodiments, the methods comprise ligating a second adapter to a second end of the SAP to produce a double-adapter-polynucleotide ligation product (DAP). In some embodiments, the methods comprise circularizing the SAP by intramolecular ligation of the SAP ends to produce a circular adapter-polynucleotide ligation product (CSAP).

In some instances, certain splint-independent and/or splint-dependent (intermolecular and/or intramolecular) ligation reactions are selected to maximize the efficiency of ligation between the adapter and polynucleotide in each ligation step, which can vary depending on the target polynucleotide (RNA or DNA) and the present target polynucleotide. In some instances, splint-independent ligation provides a higher efficiency of ligation between adapter and polynucleotide. In some instances, splint-dependent ligation provides a higher efficiency of ligation between adapter and polynucleotide. In some instances, splint-independent ligation is used to ligate the polynucleotide with the first adapter and splint-dependent ligation is used to ligate the second adapter. In some instances, splint-independent (intermolecular) ligation is used to ligate the polynucleotide with the first adapter and splint-independent (intramolecular) ligation is used to circularize the product of ligation between the first adapter and the polynucleotide. In some instances, splint-independent (intermolecular) ligation is used to ligate the polynucleotide with the first adapter and splint-dependent (intramolecular) ligation is used to circularize the product of ligation between first adapter and the polynucleotide.

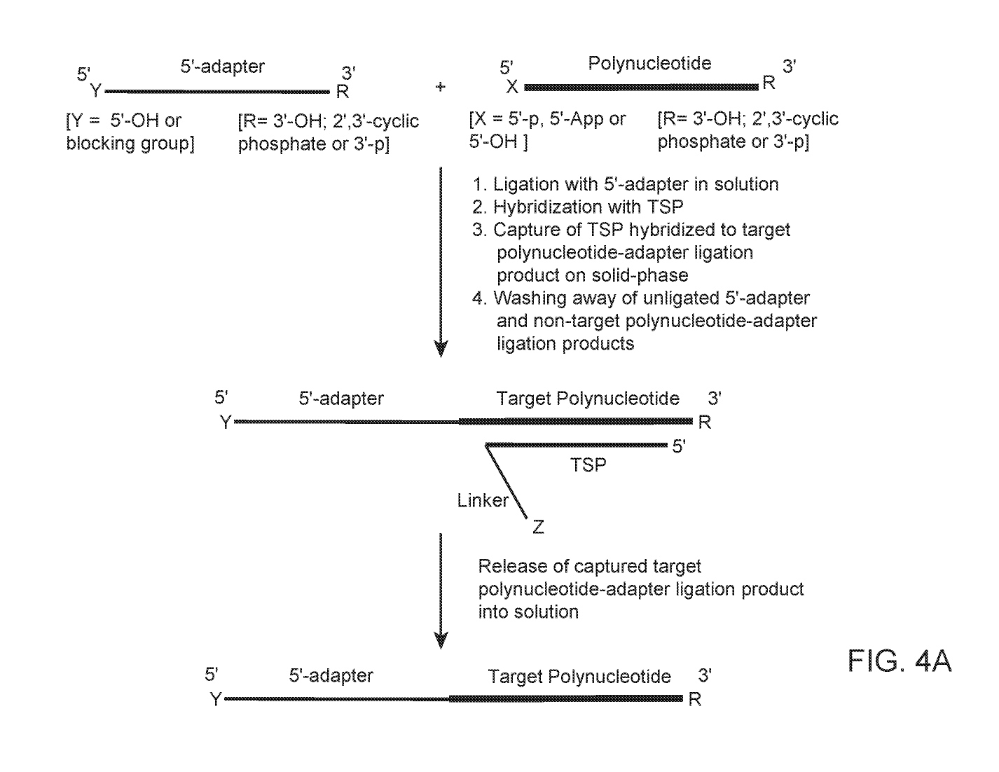

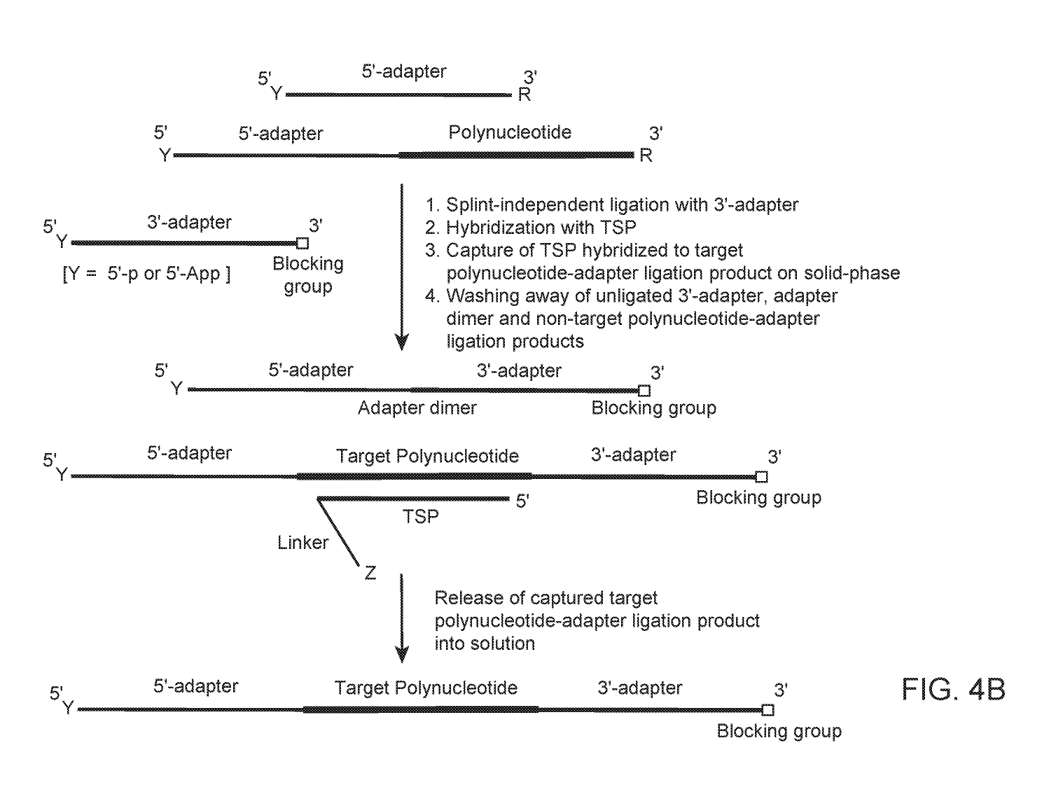

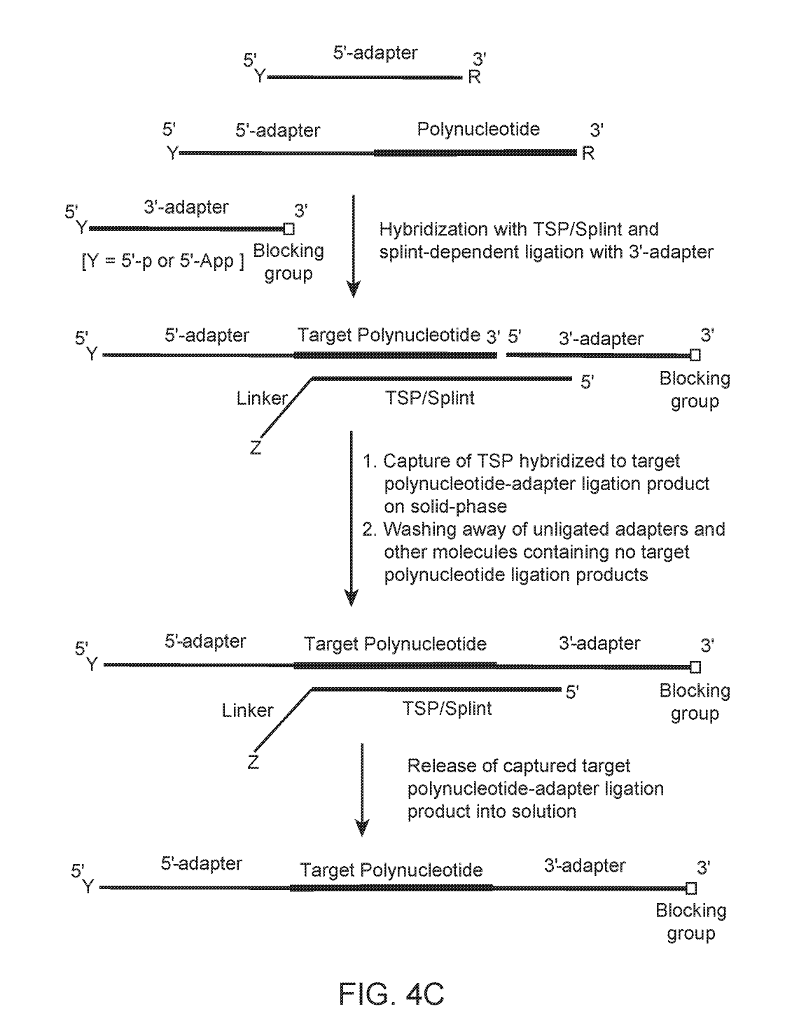

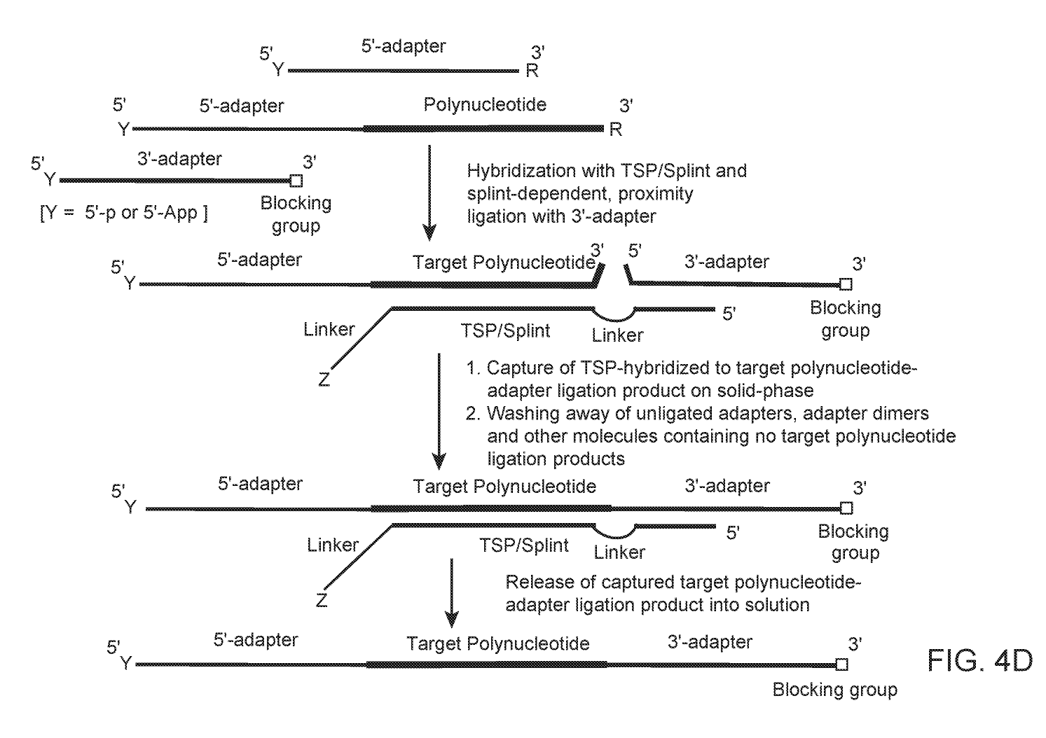

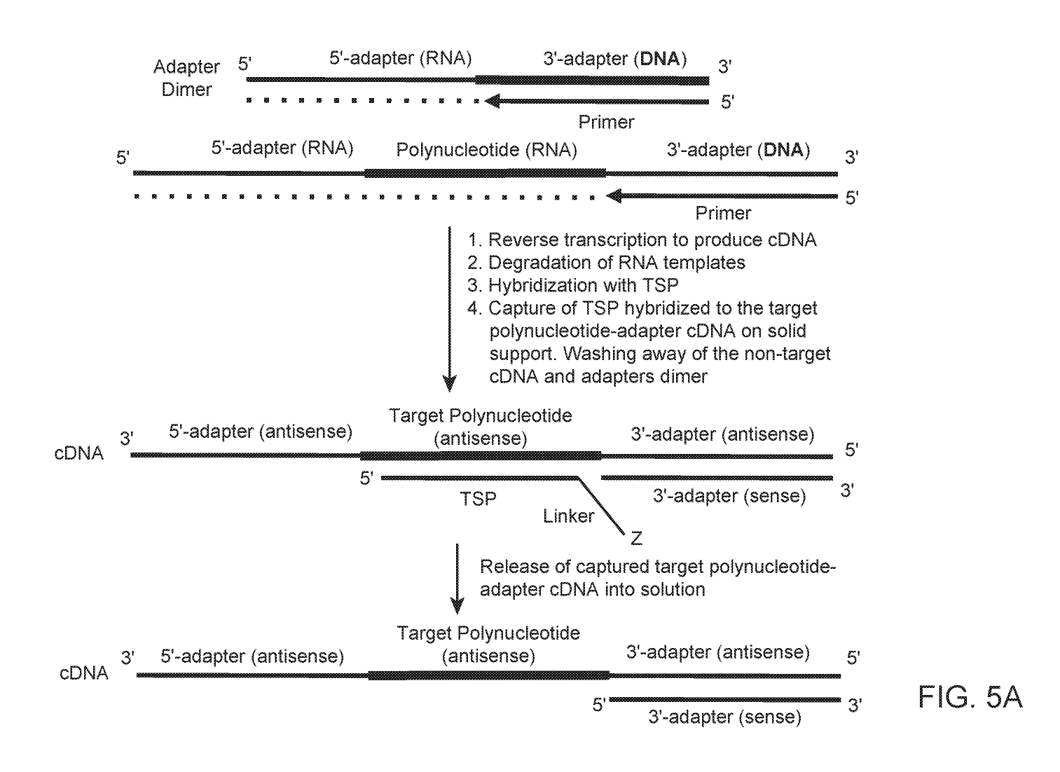

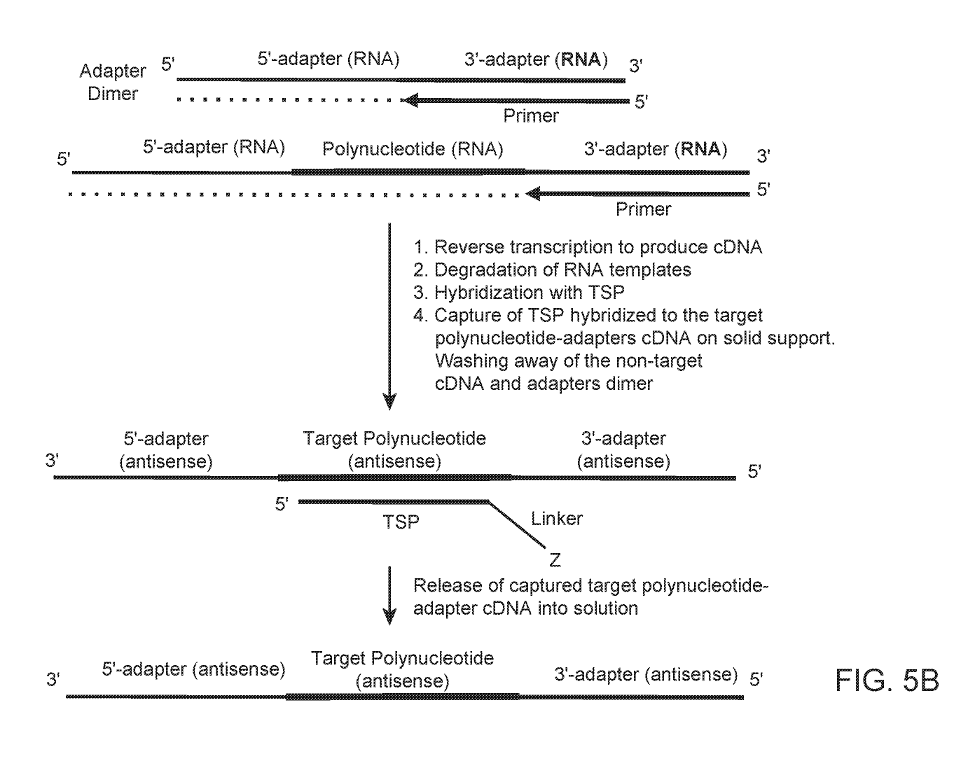

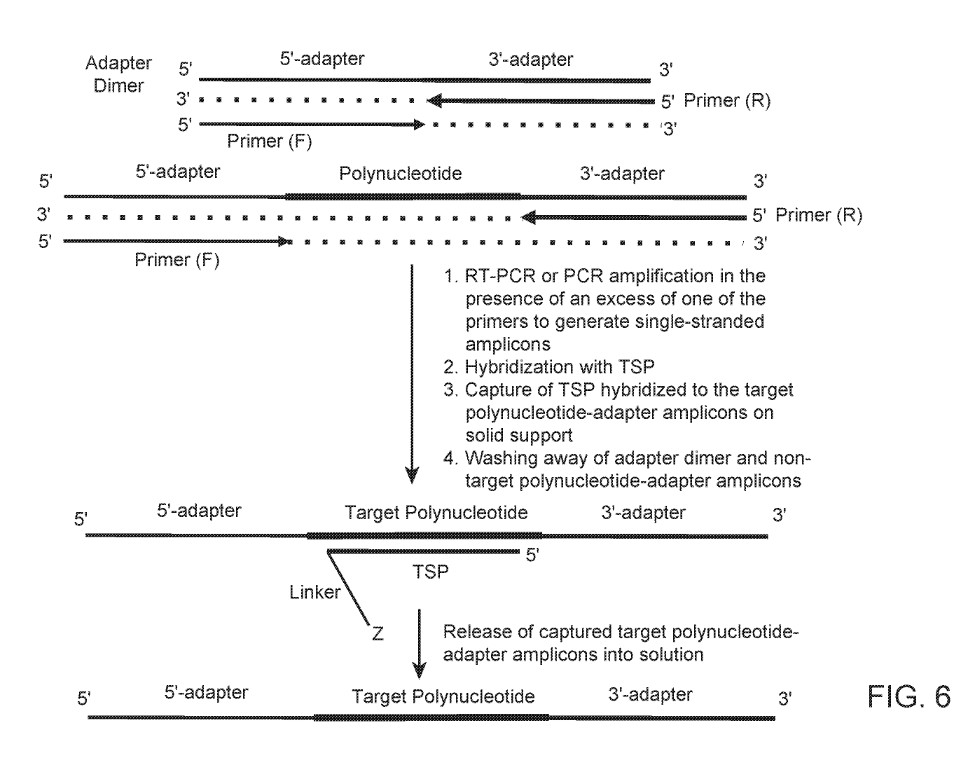

In some instances, the ligation of the second adapter is performed via splint-dependent ligation, wherein the TSP serves as both splint and capture probe. In some instances, the TSP serves as a splint, having sequences complementary to a 3′-end proximal segment of the target polynucleotide and to a 5′-proximal segment of the 3′-adapter, thereby aligning these ends head-to-tail within the duplex formed with the splint. In some instances, the TSP serving as a splint is complementary to a 5′-end proximal segment of the target polynucleotide and to a 3′-end proximal segment of the 5′-adapter, thereby aligning these ends head-to-tail within the duplex formed with the splint. In some instances, the TSP serving as a splint comprises: (i) a 3′-end proximal segment that is complementary to a 3′-end segment of the target polynucleotide; (ii) a 5′-end proximal segment that is complementary to a 5′-end proximal segment of the 3′-adapter; and (iii) a linker connecting the 3′-end proximal segment and the 5′-end segment of the TSP, wherein the linker is not complementary to one or more nucleotide(s) at the polynucleotide's 3′ end and/or at the 3′-adapter's 5′ end (See, e.g., FIG. 4D). In some instances, the TSP serving as a splint comprises: (i) a 5′-end proximal segment that is complementary to a 5′-end segment of the target polynucleotide; (ii) a 3′-end proximal segment that is complementary to a 3′-end proximal segment of the 5′-adapter; and (iii) a linker connecting the 5′-end proximal segment and the 3′-end segment of the TSP, wherein the linker is not complementary to one or more nucleotide(s) at the polynucleotide's 5′ end and at the 5′-adapter's 3′ end. The TSP's non-complementary linkers may comprise a sequence of defined nucleotides, or random nucleotide sequence, or abasic sites, or non-nucleotide residues, or combination thereafter.

In some instances, the first and/or second adapter is ligated to the polynucleotide via a splint-independent (or template-independent) intermolecular ligation using an RNA ligase selected from the group consisting of T4 RNA ligase 1 (Rnl1); T4 RNA ligase 2 (Rn12); and a T4 RNA ligase 2 (Rnl2) derivative; e.g., T4 RNA ligase 2 (1-249) truncated form or RNA ligase 2 (1-249) truncated form with the point mutation K227Q. In some instances, Rnl1 is used for ligation of both 3′- and 5′-adapters, wherein the 3′-adapter is used in 5′-adenylated (5′-App) form in the absence of ATP while the 5′-adapter is ligated in the presence of ATP. In some instances, Rnl2 or a Rnl2 derivative is used in ligation of the 3′-adapter, which is used in 5′-adenylated (5′-App) form in the absence of ATP. In contrast, Rnl1 is used in ligation of the 3′-adapter in the presence of ATP.

In some instances, only one ligase is used for ligation of an adapter. In some instances, multiple ligases are used simultaneously.

In some instances, the second adapter is attached to the polynucleotide via a splint-dependent (or template-dependent) intermolecular ligation.

In some instances, the first adapter is ligated to the 3′ end of the polynucleotide and the second adapter is ligated to the 5′ end the polynucleotide. In some instances, the first adapter is ligated to the 5′ end of the polynucleotide and the second adapter is ligated to the 3′ end of the polynucleotide.

In some instances, a single adapter is ligated to the polynucleotide's 3′-end or 5′-end via intermolecular ligation followed by circularization (intramolecular ligation) of the adapter-polynucleotide ligation product. In some instances, the circularization is performed via splint-independent (or template-independent) intramolecular ligation. In some instances, the circularization is performed via splint-dependent (or template-dependent) intramolecular ligation. In some instances, the TSP serves as a splint or template for such circularization reactions.

In some instances, the ligation of polynucleotide and adapter comprises the ligation between 5′-phosphate (5′-p) and 3′-hydroxyl (3′-OH) ends. In some instances, the polynucleotide has a 5′-hydroxyl (5′-OH) end that may be converted to 5′-p to allow ligation. A non-limiting example of the 5′-OH conversion to 5′-p is a reaction with polynucleotide kinase in the presence of ATP. In some instances, the polynucleotide has 3′-phosphate (3′-p) and/or 2′-phosphate (2′-p) ends or 2′,3′-cyclic phosphate (2′,3′>p) ends that may be converted to 3′-hydroxyl (3′-OH) and/or 2′-hydroxyl (2′-OH) ends to allow ligation. A non-limiting example of the 2′-p/3′-p conversion to 2′-OH/3′-OH is a reaction with an alkaline phosphatase or a polynucleotide kinase. A non-limiting example of the 2′,3′>p conversion to 2′-OH/3′-OH is a reaction with a polynucleotide kinase. In some instances, the ligation of polynucleotide and adapter comprises the ligation between 5′-OH and 3′-p (or 2′,3′>p) ends. In some instances, the polynucleotide has a 5′-p end that may be converted to 5′-OH to allow the ligation. A non-limiting example of 5′-p conversion to 5′-OH includes a reaction with polynucleotide kinase in the absence of ATP and/or in the presence of ADP. In some instances, 3′-phosphate (3′-p) and/or 2′-phosphate (2′-p) ends may be converted to 2′,3′-cyclic phosphate (2′,3′>p) to allow the ligation. A non-limiting example of 2′-p/3′-p conversion to 2′,3′>p is a reaction with Mth RNA ligase. The end-conversion and ligation steps may be performed in a manner selected from: a) simultaneously in a single reaction mixture; b) sequentially in a single reaction mixture; and c) sequentially in separate reaction mixtures.

In some instances, the ligating and/or circularizing by splint-independent ligation may be performed by a 3′-OH ligase (which ligates 3′-OH and 5′-phosphate ends), e.g.: T4 RNA ligase, T4 RNA ligase 1 (Rnl1), T4 RNA ligase 2 (Rnl2) or its derivatives (e.g., mutated and/or truncated versions), Mth RNA Ligase, CircLigase™ ssDNA ligase, CircLigase™ II ssDNA ligase, CircLigase™ RNA Ligase, or Thermostable RNA ligase. In some instances, the ligating and/or circularizing by splint-independent ligation may be performed by a 5′-OH ligase (which ligates 5′-OH and 3′-phosphate or 2′, 3′-cyclic phosphate ends) selected from: RNA-splicing ligase (RtcB), A. thaliana tRNA ligase (AtRNL), tRNA ligase enzyme (Trl1), and tRNA ligase (Rlg1).

In some instances, the ligating and/or circularizing by splint-dependent (or template-dependent) ligation is performed using duplex specific ligase or ligases, e.g. T4 DNA ligase, RNA ligase 2 or SplintR™ (PBCV-1) ligase. In some instances, the TSP serves as the splint or template. In some instances, an oligonucleotide other than the TSP serves as the splint or template.

In some instances, an optional, additional ligation and/or circularization step can be performed under different reaction conditions using the same or different ligase(s) if some adapter and/or polynucleotides cannot be efficiently ligated or circularized in a single step.

Ligating and/or circularizing may occur in the absence of ATP. Ligating and/or circularizing may occur in the presence of cofactors selected from: ATP, GTP, Mg²⁺, Mn²⁺, or a combination thereof.

Since circularization of the adapter-polynucleotide ligation product via intramolecular ligation is more efficient than intermolecular ligation of the 5′-adapter in standard two-adapter ligation methods, the circularization-based approach may provide effective ligation of a wider variety of polynucleotide sequences (i.e., reduced ligation bias).

In some instances, the methods comprise hybridizing the TSP to the SAP. In some instances, the methods comprise hybridizing the TSP to the target polynucleotide after the first adapter is ligated to the first end of the target polynucleotide. In some instances, the methods comprise hybridizing the TSP to the target polynucleotide directly after the first adapter is ligated to the first end of the target polynucleotide.

In some embodiments, the methods comprise ligating the second adapter to the second end of the SAP to produce the DAP. In some instances, the methods comprise hybridizing the TSP to the SAP before ligating the second adapter to the second end of the SAP. In some instances, the methods comprise hybridizing the TSP to the SAP directly before ligating the second adapter to the second end of the SAP. In some instances, the methods comprise hybridizing the TSP to the DAP. In some instances, the methods comprise hybridizing the TSP to the DAP after ligating the second adapter to the second end of the SAP. In some instances, the methods comprise hybridizing the TSP to the DAP directly after ligating the second adapter to the second end of the SAP.

In some embodiments, the methods comprise circularizing the SAP by intramolecular ligation of the SAP ends to produce the CSAP. In some instances, the methods comprise hybridizing the TSP to the SAP before circularizing the SAP to produce the CSAP. In some instances, the methods comprise hybridizing the TSP to the SAP directly before circularizing the SAP to produce the CSAP. In some instances, the methods comprise hybridizing the TSP to the CSAP. In some instances, the methods comprise hybridizing the TSP to the CSAP after circularizing the SAP. In some instances, the methods comprise hybridizing the TSP to the CSAP directly after circularizing the SAP.

In some embodiments, the methods comprise hybridizing a primer to the DAP or CSAP and extending by a polymerase to produce a complementary DNA (cDNA) or primer extension product. In some embodiments, the methods comprise amplifying the cDNA or primer extension product. For simplicity, the cDNA and amplified cDNA may be referred to as a primer extension product. In some embodiments, the methods comprise hybridizing a TSP to at least a portion of the primer extension product. In some embodiments, the methods comprise capturing the primer extension product via the TSP on a solid support to produce a captured target polynucleotide. In some embodiments, the methods comprise removing components from the sample that are not captured on the solid support. Non-limiting examples of components removed from the sample include non-target polynucleotides, solutes and inhibitors of certain enzymatic reactions that may be present in sample. In some embodiments, the methods comprise releasing the captured primer extension product into solution to produce a released primer extension product. In some embodiments, the methods comprise detecting the released primer extension product.

In some embodiments, the methods comprise hybridizing the TSP to the DAP or CSAP before hybridizing the primer. In some embodiments, the methods comprise hybridizing the TSP to the DAP or CSAP directly before hybridizing the primer. In some embodiments, the methods comprise hybridizing the TSP to the DAP or CSAP before extending the primer. In some embodiments, the methods comprise hybridizing the TSP to the DAP or CSAP directly before extending the primer. In some embodiments, the methods comprise hybridizing the TSP to the cDNA. In some embodiments, the methods comprise amplifying the cDNA. In some embodiments, the methods comprise hybridizing the TSP to the cDNA before amplifying. In some embodiments, the methods comprise hybridizing the TSP to the cDNA directly before amplifying. In some embodiments, the methods comprise hybridizing the TSP to the cDNA after amplifying. In some embodiments, the methods comprise hybridizing the TSP to the cDNA after amplifying. In some embodiments, the methods comprise hybridizing the TSP to the cDNA directly after amplifying.

Methods and compositions disclosed herein may be used for constructing or preparing libraries of polynucleotides of interest (target polynucleotides). The target polynucleotides may comprise RNA, DNA, modified RNA, modified DNA or a combination thereof. In certain embodiments, said libraries of target polynucleotide are sequencing libraries. Said sequencing libraries may be prepared using alternative approaches. In some embodiments, one approach uses a consecutive ligation of two sequencing adapters: 3′-adapter to 3′ end and 5′-adapter to 5′ end of RNA. In other embodiments, an alternative approach uses a ligation of a single combo adapter (CAD) comprising sequences of both 3′ and 5′ sequencing adapters to one end of the target RNA followed by circularization of the ligation product by intermolecular ligation of free RNA and combo adapter ends. The methods disclosed here-may further comprise depleting non-target polynucleotides and other sample components by capturing target polynucleotide-specific sequences on a solid support using target-specific probes (TSP) and washing away or removing the nontarget components that are not captured on the solid support.

The methods may comprise hybridizing a first primer comprising a sequence at least partially complementary to the 3′ or 5′-proximal segment of said first adapter or second adapter. The methods may further comprise extending the primer with a polymerase to produce a plurality of cDNAs, wherein each of the cDNAs is complementary to at least one target polynucleotide of the sample, and wherein the cDNAs are flanked by at least a portion of a sequence corresponding to or complementary to the sequencing adapter. The methods may further comprise extending the primer with a polymerase to produce a plurality of primer extension products, wherein each of the extension products is complementary to at least one target polynucleotide of the sample, and wherein the extension products are flanked by at least a portion of a sequence corresponding to or complementary to the sequencing adapter.

In some embodiments, said polymerase may be a reverse transcriptase (RNA-dependent DNA polymerase). In some embodiments, said reverse transcriptase may have or may lack an RNase H activity that cleaves an RNA template after the primer extension. In some embodiments, said reverse transcriptase may also have a DNA-dependent activity that allows primer extension on both RNA and DNA templates. In some embodiments, said reverse transcriptase may lack DNA-dependent activity and may therefore be unable to perform primer extension on a DNA template. By way of non-limiting example, the reverse transcriptase may be selected from: SuperScript® II, SuperScript® III, SuperScript® IV, ThermoScript™, Maxima™ RevertAid™; AMV, M-MuLV, PyroPhage RT, and ProtoScript® II. In some embodiments, said polymerase may be a DNA polymerase (DNA-dependent DNA polymerase). In some embodiments, said DNA polymerase may lack the RNA-dependent activity that disallows or prevents a primer extension on a RNA template. In some embodiments, said DNA polymerase may also have the RNA-dependent activity that allows a primer extension on both DNA and RNA template. By way of non-limiting example, the DNA polymerase may be selected from: DNA polymerase I, DNA polymerase I large fragment (Klenow fragment), Bst 3.0 DNA polymerase, Tth or rTth DNA polymerase, Taq and Platinum Taq polymerases.

In some embodiments, methods further comprise amplifying the plurality of cDNAs using a second primer and a third primer, wherein the sequence of the third primer is at least partially complementary to the 5′-proximal segment of said adapter, to produce amplicon(s) comprising a sequencing library.

After the library preparations comprising processed target polynucleotide sequences, the target polynucleotides may then be detected, identified and quantified by using known in art methods including (but not limited to): sequencing, microarrays, RT-qPCR, qPCR, PCR arrays, or digital PCR.

In the some embodiments, methods disclosed herein comprise detecting target polynucleotides by sequencing. If not eliminated, the non-target polynucleotide sequences may saturate the sequencing reads and, therefore, reduce the number of the sequencing reads related to target polynucleotides. The eliminating (depleting or reducing) the non-target polynucleotides from the sequencing libraries may increase the sensitivity and reduce a cost of target polynucleotide sequencing.

In some embodiments of the invention, the depleting unrelated (non-target) polynucleotides either before or in a process of the library preparation is performed by multiplex hybridization of target-specific probe (TSP) to each target polynucleotide and capture of TSP-polynucleotide duplexes on a solid support following by washing of non-target polynucleotides and other solutes (including those that may interfere with downstream reactions during the library preparation). The captured target polynucleotides or processed target polynucleotides (such as adapter-polynucleotide ligation products, products of circularizing of adapter-polynucleotide ligation products as well as products of RT and PCR) are then released into solution by dissociating from TSP before the next processing step. The TSP-assisted capture of the target polynucleotides or processed target polynucleotides may also be used for concentrating these polynucleotides from the diluted solutions.

In some embodiments, methods comprise ligating an additional nucleic acid fragment, such as an adapter, bar code or probe, to the target polynucleotide. In some embodiments, an adapter comprises one or more priming sites, barcodes, or sequencing linkers. In some embodiments, TSPs are ligated to haptens. Exemplary haptens may include, biotin, digoxigenin, peptide tags, or other chemical moiety for capture. In some embodiments, ligation occurs on a solid support. In some embodiments, the TSPs comprise modified nucleic acids. In some embodiments, ligation occurs in solution. In some embodiments, TSPs are captured on a solid support, such as a magnetic bead or other suitable surface. In some embodiments, adapters-polynucleotide constructs are circularized. In some embodiments, adapter-polynucleotide constructs are reverse-transcribed to generate a cDNA library. In some embodiments, cDNA libraries are further amplified. In some embodiments, adapter-polynucleotide constructs are detected with a method such as sequencing. In some embodiments, detection comprises identifying or quantifying adapter-polynucleotide constructs or their amplification products. In some embodiments, cDNA libraries are detected with a method such as sequencing. In some embodiments, the sequencing method is NGS.

### Samples

Provided herein are methods for detecting a target polynucleotide in a biological sample. In some instances, the methods comprise in vivo detection (e.g., detection directly in biological samples). In some instances, the methods comprise in vitro or ex vivo detection (e.g., detection of a target polynucleotide in a pool of isolated total nucleic acids). In some embodiments, the nucleic acid sample is DNA, messenger RNA, or miRNA, or a combination thereof.

Biological samples include biological tissues or fluids. Non-limiting exemplary biological samples are blood, plasma, urine, saliva, sweat, buccal cells, cerebrospinal fluid. In some embodiments, samples are processed prior to analysis. In some embodiments, biological samples are obtained from a single source, or from multiple sources. In some embodiments, samples are obtained at different time points. In some embodiments, the number of samples at least 1, 2, 3, 5, 10, 20, 50, 100, or more than 100 samples. In some embodiments, the number of samples is about 1 to about 10 samples, about 2 to about 20 samples, about 10 to about 25 samples, about 25 to about 75 samples, or about 10 to about 100 samples.

The biological samples may comprise a lysate of biological fluid (biofluid), cell fresh tissue biopsy, or formalin-fixed paraffin-embedded (FFPE) blocks. The target polynucleotide from the samples may be analyzed without prior isolation of total RNA and/or DNA.

Also provided herein are methods for detecting a target polynucleotide in artificial or man-made (synthetic) samples. Non-limiting examples of artificial samples include: pools of synthetic polynucleotides (e.g., miRXplore™ Universal pool, which contains equal amounts of 962 synthetic miRNAs, from Miltenyi Biotec); artificial pools of polynucleotides isolated from different biological samples (e.g., Universal miRNA Reference Kit, which contains miRNAs from different human tissues and cell types along with mRNAs, lncRNAs and piRNAs, from Agilent); and biological samples containing spiked-in synthetic polynucleotides as normalization and/or quantification controls. Also provided herein are methods for detecting target polynucleotides using very low inputs of sample polynucleotides using an addition of carrier polynucleotides (natural or synthetic). Non-limiting examples of very low inputs include: sample polynucleotides from single cells and cell-free circulating polynucleotides from biofluids (e.g., plasma, serum, urine or saliva).

### Target Polynucleotides

Provided herein are methods for detecting a target polynucleotide in a biological sample containing a plurality of polynucleotides. The target polynucleotide may comprise RNA. The target polynucleotide may consist essentially of RNA. The target polynucleotide may comprise naturally occurring RNA. The target polynucleotide may consist essentially of naturally occurring RNA. The target polynucleotide may comprise synthetic RNA. The target polynucleotide may consist essentially synthetic RNA. The target polynucleotide may comprise naturally occurring RNA and synthetic RNA. The target polynucleotide may consist essentially of naturally occurring RNA and synthetic RNA. The target polynucleotide may comprise small RNA. The target polynucleotide may consist essentially of small RNA. The target polynucleotide may comprise a small fragment of a large RNA. The target polynucleotide may consist essentially of a small fragment of a large RNA. The target polynucleotide may comprise circular RNA. The target polynucleotide may consist essentially of circular RNA, in which case it is cleaved or fragmented before the adapter ligation. The target polynucleotide may comprise single-stranded RNA. The target polynucleotide may consist essentially of single-stranded RNA. The target polynucleotide may comprise double-stranded RNA. The target polynucleotide may consist essentially of double-stranded RNA. In some instances, double-stranded RNA may be converted to single-stranded RNA before detecting.

The target polynucleotide may comprise DNA. The target polynucleotide may consist essentially of DNA. The target polynucleotide may comprise naturally occurring DNA. The target polynucleotide may consist essentially of naturally occurring DNA. The target polynucleotide may comprise synthetic DNA. The target polynucleotide may consist essentially of synthetic DNA. The target polynucleotide may comprise naturally occurring DNA and synthetic DNA. The target polynucleotide may consist essentially of naturally occurring DNA and synthetic DNA. The target polynucleotide may comprise small DNA or small fragments of large DNAs. The target polynucleotide may consist essentially of small DNA or small fragments of large DNAs. The target polynucleotide may comprise circular DNA. The target polynucleotide may consist essentially of circular DNA. The circular DNA may be cleaved or fragmented before the adapter ligation. The target polynucleotides may comprise single-stranded DNA. The target polynucleotides may consist essentially of single-stranded DNA. The target polynucleotide may comprise double-stranded DNA. The target polynucleotide may consist essentially of double-stranded DNA. In some instances, double-stranded DNA may be converted to single-stranded DNA before detecting.

The term “small RNA” generally refers to RNA or RNA fragments about 250 nucleotides or less. In some embodiments, the small RNA does not possess more than about 250 nucleotides. In some embodiments, the small RNA does not possess more than 250 nucleotides. In some embodiments, the small RNA does not comprise more than about 250 nucleotides. In some embodiments, the small RNA does not comprise more than 250 nucleotides. In some embodiments, the small RNA is not more than 250 nucleotides in length. In some embodiments, the small RNA is not more than about 250 nucleotides in length. In some embodiments, the small RNA does not consist of more than 250 nucleotides. In some embodiments, the small RNA does not consist of more than about 250 nucleotides. In some embodiments, the small RNA consists essentially of 250 nucleotides or less. In some embodiments, the small RNA consists essentially of about 250 nucleotides or less. In certain embodiments, the small RNA contains no more than 210 nucleotides, no more than 220 nucleotides, no more than 230 nucleotides, no more than 240 nucleotides, or no more than 250 nucleotides. In some embodiments, the small RNA contains about 1 nucleotide to about 250 nucleotides, about 10 nucleotides to about 250 nucleotides, about 50 nucleotides to about 250 nucleotides, about 100 nucleotides to about 200 nucleotides, or about 200 nucleotides to about 250 nucleotides. As used herein, nucleotides of the small RNA are generally ribonucleotides. In some embodiments, the ribonucleotides may be chemically modified ribonucleotides. In some embodiments, the target polynucleotides are small RNAs, RNA or DNA fragments of 250 or fewer nucleotides in length. Large polynucleotides (e.g., “large RNA) comprise more than 250 nucleotides. Accordingly, adapter-target ligation products may be as long as about 350 nucleotides. In some embodiments, the small DNA fragments are tumor-derived, cell-free single-stranded DNAs of ≤100 nt.

In some embodiments, the small RNAs are messenger RNAs (mRNA) or fragments thereof. The fragments may have a length of small RNAs described herein. In some embodiments, the said small RNAs are microRNAs (miRNAs). In some embodiments, the said RNA fragments are tRNA fragments.

In some embodiments, the target small RNAs and/or small fragments of large RNAs are converted into small RNA sequencing libraries and then detected by next-generation sequencing (NGS), a.k.a. small RNA-Seq.

The non-target RNAs commonly preset in biological samples may include fragments of ribosomal RNAs, tRNAs as well as over-represented small RNAs. Another class of non-target polynucleotides is represented by so-called “adapter dimers” that may be formed during the preparation of sequencing libraries.

In some instances, the target polynucleotide has 5′-OH end that may be converted to 5′-p to allow the ligation with an adapter by a 3′-OH ligase. Non-limiting examples of the 5′-OH conversion to 5′-p includes reaction with T4 polynucleotide kinase or thermostable polynucleotide kinase in the presence of ATP. In some instances, the target polynucleotide has 3′-phosphate (3′-p) and/or 2′-phosphate (2′-p) ends or 2′,3′-cyclic phosphate (2′,3′>p) ends that may be converted to 3′-hydroxyl (3′-OH) and/or 2′-hydroxyl (2′-OH) ends to allow the ligation with an adapter by a 3′-OH ligase. Non-limiting example of the 2′-p/3′-p conversion to 2′-OH/3′-OH includes a reaction with an alkaline phosphatase or a polynucleotide kinase. The said alkaline phosphatase may be selected from: Calf Intestinal Phosphatase (CIP), Shrimp Alkaline Phosphatase (rSAP), APex™ Heat-labile alkaline phosphatase and Antarctic Phosphatase. Non-limiting examples of the 2′,3′>p conversion to 2′-OH/3′-OH includes a reaction with a polynucleotide kinase. Non-limiting examples of the 3′-OH ligases (which ligate 3′-OH and 5′-p ends) include: T4 RNA ligase, T4 RNA ligase 1 (Rnl1), T4 RNA ligase 2 (Rnl2) or its derivatives (e.g., mutated and/or truncated versions), Mth RNA Ligase, CircLigase™ ssDNA ligase, CircLigase™ II ssDNA ligase, CircLigase™ RNA Ligase, or Thermostable RNA ligase.

In some instances, the target polynucleotide has 5′-p end that may be converted to 5′-OH to allow the ligation by a 5′-OH ligase. Non-limiting example of the 5′-p conversions to 5′-OH includes a reaction with polynucleotide kinase in the absence of ATP and/or in the presence of ADP. In some instances, the target polynucleotide has 3′-phosphate (3′-p) and/or 2′-phosphate (2′-p) ends that may be converted to 2′,3′-cyclic phosphate (2′,3′>p) to allow the ligation by a 5′-OH ligase. Non-limiting examples of the 2′-p/3′-p conversions to 2′,3′>p includes a reaction with Mth RNA ligase. Non-limiting examples of the 5′-OH ligases (which ligate 5′-OH and 3′-p or 2′,3′>p ends) include: RNA-splicing ligase (RtcB), A. thaliana tRNA ligase (AtRNL), tRNA ligase enzyme (Trl1), and tRNA ligase (Rlg1).

### Target Specific Probes

The methods, compositions, and kits disclosed herein comprise target-specific oligonucleotide probes (TSPs). As used herein, a “target-specific probe” (TSP) is an oligonucleotide that can hybridize to a target polynucleotide or both a target polynucleotide and adapter as disclosed herein. The TSPs disclosed herein may comprise non-specific linker(s), which is (are) neither complementary nor corresponding to the target polynucleotide and/or adapters. The non-specific linker disclosed herein may comprise a sequence of defined nucleotides, or random nucleotide sequence, or abasic sites, or non-nucleotide residues, or combination thereafter. In some instances, the non-specific linker disclosed herein may have a length equivalent to an oligonucleotide of 1 to 20 nucleotides. The TSPs and the non-specific linkers disclosed herein may comprise one or more nucleotide residues selected from: a deoxyribonucleic acid (DNA), a ribonucleic acid (RNA), and a chemically modified derivative of DNA or RNA. Non-limiting examples of chemically modified derivatives include 2′-OMe, 2′-methoxyethyl (2′-MOE) or 2′-fluoro (2′-F), locked nucleic acids (LNA), chemically modified nucleobase derivatives of DNA or RNA, abasic sites, a mimetic of DNA or RNA, peptide nucleic acid (PNA), morpholino-based nucleotides, non-nucleotide linkers and any combination thereof. In some instances, the TSPs further comprise one or more non-natural analogs.

In some instances, the TSP comprises a sequence that is complementary to the sequence of the target polynucleotide. The sequence of the TSP can be at least about 50% to about 100% complementary to the sequence of the target polynucleotide. In some instances, the sequence of the TSP is at least about 70% complementary to the sequence of the target RNA. In other instances, the sequence of the TSP is at least about 75% complementary to the sequence of the target polynucleotide. Alternatively, the sequence of the TSP is at least about 80% complementary to the sequence of the target polynucleotide. The sequence of the TSP can be at least about 85% complementary to the sequence of the target polynucleotide. In some instances, the sequence of the TSP is at least about 87% complementary to the sequence of the target polynucleotide. In other instances, the sequence of the TSP is at least about 90% complementary to the sequence of the target polynucleotide. Alternatively, the sequence of the TSP is at least about 95% complementary to the sequence of the target polynucleotide. The sequence of the TSP can be at least about 97% complementary to the sequence of the target polynucleotide. In some instances, the sequence of the TSP is at least about 98% complementary to the sequence of the target polynucleotide. In other instances, the sequence of the TSP is at least about 99% complementary to the sequence of the target polynucleotide. The TSPs for use in the methods, compositions, and kits disclosed herein may comprise one or more blocking groups at their 5′ end and/or 3′ end. In some instances, the blocking group on the TSP reduces and/or prevents ligation to the 5′ and/or 3′ end of the TSP. In some instances, the TSP comprises a blocking group at its 5′ end (e.g., 5′-blocking group). In other instances, the TSP comprises a blocking group at its 3′ end (e.g., 3′-blocking group). Alternatively, the TSP comprises a blocking group at its 5′ end and its 3′ end.

In some instances, the blocking group comprises a termination group that is a 3′-amino; a 2′,3′-dideoxy nucleoside (ddN); a 3′-inverted (3′-3′) deoxynucleoside (idN); a 3′-inverted abasic site; or a 3′-non-nucleoside linker (n-linker). In some embodiments, the TSP comprises a blocking group at its 5′ end that prevents its phosphorylation, e.g., a 5′-OMe or a non-nucleotide linker. In some embodiments, the TSP comprises one or more residues that cannot be replicated by DNA polymerase; e.g., an abasic site(s) or nucleoside(s) with 2′-OMe or 2′-F modifications.

In some instances, the 3′ blocking group on the TSP reduces and/or prevents extension of the 3′ end of TSP. In some instances, the 3′ blocking group on the TSP reduces and/or prevents extension of the 3′ end of TSP by a reverse transcriptase. In other instances, the 3′ blocking group on the TSP reduces and/or prevents extension of the 3′ end of TSP by a DNA polymerase.

In some embodiments, the TSP is hybridized to at least a part of target polynucleotide sequence (sense or antisense) but not to the adapter sequence. The latter allows simultaneously capturing and detecting both target polynucleotide sequences and their closely related sequence variants that differ by small number of nucleotides. In case of target miRNAs such variants may include isomiRs and isoforms.

In other embodiments, the TSP may be hybridized to at least a part of both target polynucleotide and adapter sequences, wherein the TSP serves as both a capture probe and a splint to allow the splint-dependent ligation of target polynucleotide with an adapter. In some instances, the TSP serving as a splint is complementary to a 3′-end-proximal segment of the target polynucleotide and to a 5′-end-proximal segment of the 3′-adapter, thereby aligning these ends head-to-tail within the duplex formed with the splint. In some instances, the TSP serving as a splint is complementary to a 5′-end-proximal segment of the target polynucleotide and to a 3′-end-proximal segment of the 5′-adapter, thereby aligning these ends head-to-tail within the duplex formed with the splint. Such TSP designs may allow the detection of specific sequence variants of the target polynucleotides with higher sequence specificity.

In some instances, the TSP serving as both a capture probe and a splint comprises: (i) a 3′-end proximal segment, which is complementary to a 3′-end segment of the target polynucleotide; (ii) a 5′-end proximal segment, which is complementary to a 5′-end-proximal segment of the 3′-adapter; and (iii) a linker connecting the 3′-end proximal segment and the 5′-end segment of the TSP, wherein the linker is not complementary to one or more nucleotide(s) at the 3′ end of the polynucleotide and at the 5′ end of the 3′-adapter. In some instances, the TSP serving as a splint comprises: (i) a 5′-end proximal segment, which is complementary to a 5′-end segment of the target polynucleotide; (ii) a 3′-end proximal segment, which is complementary to a 3′-end proximal segment of the 5′-adapter; and (iii) a linker connecting the 5′-end proximal segment and the 3′-end segment of the TSP, wherein the linker is not complementary to one or more nucleotide(s) at the 5′ end of the polynucleotide and at the 3′ end of the 5′-adapter. Such TSP designs may allow more efficient ligation between a defined adapter end and target polynucleotides having variable nucleotides at their ends. The latter allows the simultaneous capture and detection of both target polynucleotide sequences and their closely related sequence variants that differ by small number of nucleotides at the ends of the target polynucleotide. In some other instances, hybridizing of the TSP comprises hybridizing two or more TSP oligonucleotides to the same product produced in step (a) and/or (b).

In some embodiments, the TSP-assisted capture step(s) may be applied to purify one or more intermediate and/or final products of the library preparation for either the two-adapter ligation approach or to the single-adapter ligation and circularization approach.

In other embodiments, the TSPs may be also used to capture, purify and concentrate target polynucleotides from biological or artificial samples prior to the library construction. In some embodiments, libraries of TSPs may comprise at least 1, 2, 5, 10, 20, 50, 100, 200, 500, 1000, 2000, 5000, 10,000, or more than 10,000 TSPs. In some embodiments, libraries of TSPs may comprise about 1 to about 100 TSPs, about 10 to about 200 TSPs, about 100 to about 500 TSPs, about 200 to about 1000 TSPs, about 500 to about 500 TSPs, about 500 to about 5000 TSPs, or about 1000 to about 10,000 TSPs.

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Adapters

The methods, compositions, and kits disclosed herein may comprise one or more adapters. In some instances, the one or more adapters are ligated (or attached) to a plurality of sample polynucleotides, wherein the sample polynucleotides comprise target polynucleotides and non-target polynucleotides present in a sample. Alternatively, or additionally, the one or more adapters comprise a linker, hapten, tag, probe, label, or a combination thereof. The adapters disclosed herein may comprise one or more deoxyribonucleic acid (DNA), ribonucleic acid (RNA), modified nucleic acid and non-nucleic acid residues. Non-limiting examples of modified residues include a deoxyuridine (dU), an inosine (I), a deoxyinosine (dI), an Unlocked Nucleic Acid (UNA), a Locked Nucleic Acid (LNA) comprising a sugar modification, a Peptide Nucleic Acid (PNA), an abasic linkers (e.g., dSpacer) , and a nucleic acid residue with a modification selected from: a 5-nitroindole base modification, a 2′-phosphate (2′-p), a 2′-NH₂, a 2′-NHR, a 2′-OMe, a 2′-O-alkyl, a 2′-methoxyethoxy (MOE), a 2′-F, a 2′-halo, a phosphorothioate (PS), and a disulfide (S—S) internucleotide bond modification.

In some instances, the length of the adapter is between about 1 to about 100 nucleotides. In other instances, the length of the adapter is between about 10 to about 100 nucleotides. Alternatively, the length of the adapter is between about 20 to about 100 nucleotides. The length of the adapter can be between about 30 to about 100 nucleotides. In some instances, the length of the adapter is between about 40 to about 100 nucleotides. In other instances, the length of the adapter is between about 50 to about 100 nucleotides. Alternatively, the length of the adapter is between about 10 to about 90 nucleotides. The length of the adapter is between about 10 to about 80 nucleotides. In some instances, the length of the adapter is between about 10 to about 70 nucleotides. In other instances, the length of the adapter is between about 20 to about 80 nucleotides. Alternatively, the length of the adapter is between about 20 to about 70 nucleotides. The length of the adapter can be between about 20 to about 60 nucleotides. In some instances, the length of the adapter is between about 20 to about 50 nucleotides. In other instances, the length of the adapter is between about 20 to about 40 nucleotides. Alternatively, the length of the adapter is between about 30 to about 60 nucleotides. The length of the adapter is between about 30 to about 50 nucleotides.

In some instances, the length of the adapter is at least about 10 nucleotides. In other instances, the length of the adapter is at least about 20 nucleotides. Alternatively, the length of the adapter is at least about 30 nucleotides. The length of the adapter can be between about 40 nucleotides. In some instances, the length of the adapter is at least about 50 nucleotides. In other instances, the length of the adapter is at least about 60 nucleotides. Alternatively, the length of the adapter is at least about 70, 75, 80, 85, 90, 95, or 100 nucleotides.

In some instances, the length of the adapter is less than about 70 nucleotides. In other instances, the length of the adapter is less than about 60 nucleotides. Alternatively, the length of the adapter is less than about 55 nucleotides. The length of the adapter can be between about 50 nucleotides. In some instances, the length of the adapter is less than about 45 nucleotides. In other instances, the length of the adapter is less than about 30 nucleotides.

The adapters as disclosed herein can comprise a sequence that is not substantially complementary to the sequences of the target polynucleotides. In some instances, less than about 50% of the adapters can hybridize to the target polynucleotide or derivative thereof. In other instances, less than about 40% of the adapters can hybridize to the target polynucleotide or derivative thereof. Alternatively, less than about 30% of the adapters can hybridize to the target polynucleotide or derivative thereof. In other instances, less than about 20% of the adapters can hybridize to the target polynucleotide or derivative thereof. In some instances, less than about 10% of the adapters can hybridize to the target polynucleotide or derivative thereof. In other instances, less than about 5% of the adapters can hybridize to the target polynucleotide or derivative thereof. Alternatively, less than about 2% of the adapters can hybridize to the target polynucleotide or derivative thereof. In some instances, less than about 1% of the adapters can hybridize to the target polynucleotide or derivative thereof.

In some instances, the adapters disclosed herein can be single-stranded. In some instances, the adapters can be double-stranded and have terminal overhangs of about 3-to-12 nucleotides that are complementary to target polynucleotide ends, wherein said terminal overhangs comprise defined or randomized nucleotide sequences.

In some instances, the adapters disclosed herein further comprise a sequence for cloning. In some instances, the adapters disclosed herein further comprise a sequence for cloning, concatamerization and conventional Sanger sequencing.

In some instances, the adapters as disclosed herein are for next-generation sequencing methods and further comprise a primer sequence for reverse transcription (RT) by a reverse transcriptase and/or PCR amplification. In some instances, the primer sequence can be used for extension by a DNA polymerase. In other instances, the primer sequence can be used for PCR amplification. Alternatively, or additionally, the primer sequence can be used for sequencing.

In some embodiments, the adapters disclosed herein can further comprise a sequence that is compatible with a workflow for preparation of sequencing libraries and specific sequencing methods. In some instances, the said sequencing method may be selected from: Sanger sequencing, second- or next-generation sequencing (NGS), and third-generation sequencing or single-molecule sequencing. In some instances, the adapters further comprise a sequence that is compatible with an amplification reaction. Alternatively, or additionally, the adapter further comprises a sequence that is compatible with a reverse transcription reaction.

In some embodiments, the adapters disclosed herein can further comprise a sequence that is compatible with microarray- or bead-based detection of target polynucleotides. In some embodiments, the adapters disclosed herein can further comprise a sequence that is compatible with detection of target polynucleotides by RT-qPCR, qPCR, PCR arrays or digital PCR.

In some instances, a single adapter may be ligated to one end (5′ or 3′) of a polynucleotide. In some instances, a single adapter may be ligated to one end (5′ or 3′) of a polynucleotide when the TSP is hybridized before ligation. In some instances, two adapters may be ligated to a polynucleotide, wherein a first adapter is ligated to one end of the polynucleotide and a second adapter is ligated to the other end. The adapters can be attached to the 5′ end of a polynucleotide (i.e., 5′-adapter) to produce a 5′-end adapter-ligated polynucleotide. Alternatively, or additionally, the adapters are attached to the 3′ end of a polynucleotide (i.e., 3′-adapter) to produce a 3′-end adapter-ligated polynucleotide. In some instances, the adapters are added to the 5′ end and the 3′ end of a polynucleotide to produce a 5′-end and 3′-end adapter ligated a polynucleotide. The 5′-adapter and the 3′-adapter can be attached simultaneously. In other instances, the 5′-adapter and the 3′-adapter are attached sequentially. For example, the 5′-adapter is attached to a polynucleotide prior to attachment of the 3′-adapter to a polynucleotide. In another embodiment, the 5′-adapter is attached to a polynucleotide after attachment of the 3′-adapter to a polynucleotide. As used herein, the term “adapter-ligated target polynucleotide” refers to a target polynucleotide ligated to an adapter and can comprise 5′-end adapter-ligated target polynucleotides, 3′-end adapter-ligated target polynucleotides, and 5′-end and 3′-end adapter-ligated target polynucleotides.

The methods, compositions, and kits disclosed herein can comprise attachment of one or more adapters to a polynucleotide. Attachment of the one or more adapters to a polynucleotide can comprise conducting an enzymatic or chemical ligation reaction to attach the one or more adapters to a polynucleotide.

In some embodiments, the 5′-adapters comprise a 3′-end group that is a 3′-hydroxyl (3′-OH). In certain such embodiments, 5′-adapters comprise a 5′-end group that is a 5′-hydroxyl (5′-OH) or 5′-phosphate (5′-p). In certain such embodiments, 5′-adapters having the 5′-OH are ligated first to a polynucleotide and then are 5′-phosphorylated by polynucleotide kinase. In some embodiments, oligonucleotide adapters are ligated to the 3′ end of a polynucleotide to form 3′ end adapter-ligated polynucleotide. In some such embodiments, the 3′-adapters comprise a 5′-end group that is a 5′-phosphate (5′-p) or a 5′,5′-adenyl pyrophosphoryl cap (5′-rApp or App). The latter are also called pre-adenylated adapters.

In certain such embodiments, the 3′-adapter comprises an irreversibly blocked 3′-end. The irreversibly blocked 3′ end may comprise a termination group selected from: a 3′-amino; a 2′,3′-dideoxy nucleoside (ddN); a 3′-inverted (3′-3′) deoxynucleoside (idN); 3′-amino (3′-NH₂) a 3′-inverted abasic site; a 3′-non-nucleoside linker (n-linker), or 3′ Biotin-TEG linker. In certain such embodiments, the 5′-adapter comprises an irreversibly blocked 5′-end. The irreversibly blocked 3′ end may comprise a termination group selected from: 5′-O-Methyl (5′-OMe), 5′-amino (5′-NH₂). Blocking of an end prevents intramolecular self-ligation (circularization) and intermolecular self-ligation (multimerization or concatamerization) of the adapters as well as formation of 5′-adapter-3′-adapter ligation products (also referred as “adapter dimer”) containing no polynucleotide insert.

The terminal residues of the adapters may comprise a reversible blocking group. The reversible blocking group may be a 3′-end-blocking group. Said 3′-end-blocking group may be selected from: 3′-p, 2′,3′>p (or >p), 3′-O-(α-methoxyethyl) ether, and 3′-O-isovaleryl ester. The reversible blocking group may be a 5′-end-blocking group. Said 5′-end-blocking group may be selected from: 5′-OH, 5′-p, 5′-triphosphate (5′-ppp), 5′-diphosphate (5′-pp) and a 5′-cap structure. The reversible blocking groups perform similarly to the irreversible blocking groups in the ligation of the first (or single) adapter to a polynucleotide but then allow the circularization of such adapter-ligation product after activation. Said activation may be performed by enzymatic, chemical or photochemical conversion of the reversible blocking groups to ligatable groups. In some embodiments, the ligatable adapter groups are selected from: 5′-p and 3′-OH; 5′-OH and 3′-p (or 2′,3′>p).

In some embodiments, a single adapter is attached to the 3′ or 5′ end of a polynucleotide via intermolecular ligation to produce an adapter-polynucleotide ligation product. In some instances, the adapter-polynucleotide ligation product may then be circularized via intramolecular ligation. In some instances, the circularization is performed via splint-independent (or template-independent) intramolecular ligation. In some instances, the circularization is performed via splint-dependent (or template-dependent) intramolecular ligation. In some instances, the TSP serves as a splint or template for such circularization reactions.

In some embodiments, the 5′-adapter and/or 3′-adapter is attached to a polynucleotide via a splint-independent (or template-independent) ligation reaction. In some instances, the adapter ligation and/or circularization of the adapter-polynucleotide ligation product by splint-independent ligation may be performed by a 3′-OH ligase (which ligates 3′-OH and 5′-phosphate ends), e.g.: T4 RNA ligase, T4 RNA ligase 1 (Rnl1), T4 RNA ligase 2 (Rnl2) or Rnl2 derivatives (e.g., truncated and /or mutated versions: T4 Rnl2tr, T4 Rnl2tr K227Q, T4 Rnl2tr KQ or T4 Rnl2tr R55K), Mth RNA Ligase, CircLigase™ ssDNA ligase, CircLigase™ II ssDNA ligase, CircLigase™ RNA Ligase, or Thermostable RNA ligase. In some instances, the adapter ligation and/or circularization of the adapter-polynucleotide ligation product by splint-independent ligation may be performed by a 5′-OH ligase (which ligates 5′-OH and 3′-phosphate or 2′,3′-cyclic phosphate ends) selected from: RNA-splicing ligase (RtcB), A. thaliana tRNA ligase (AtRNL), tRNA ligase Trl1, and tRNA ligase Rlg1.

In some instances, the adapter ligation and/or circularization of the adapter-polynucleotide ligation product by splint-dependent (or template-dependent) ligation is performed using duplex specific ligase or ligases, e.g., T4 DNA ligase, RNA ligase 2 or SplintR™ (PBCV-1) ligase. In some instances, the TSP serves as the splint or template. In some instances, an oligonucleotide other than the TSP serves as the splint or template.

In some instances, an optional, additional ligation and/or circularization step can be performed under different reaction conditions using the same or different ligase(s) if some adapter and/or polynucleotides cannot be efficiently ligated or circularized in a single step.

Ligating and/or circularizing may occur in the absence of ATP. Ligating and/or circularizing may occur in the presence of cofactors selected from: ATP, GTP, Mg²⁺, Mn²⁺ or combinations thereof.

In some embodiments, the adapter is capable of being ligated to a single-stranded polynucleotide. In some embodiments, the adapter may be ligated to a single-stranded polynucleotide resulting from denaturation of a double stranded polynucleotide. In some embodiments, the adapter may be ligated to a double-stranded polynucleotide.

In some embodiments, a composition of 5′-adapter and/or 3′-adapter allows detection of the adapter-ligated target polynucleotides using microarray- or bead-based methods. In some embodiments, the 5′-adapter and/or 3′-adapter comprise one or more hapten(s) or ligand(s) that can be conjugated with signal or signal-generating moieties. In certain embodiments, the 5′-adapter and/or 3′-adapter directly comprise signal or signal-generating moieties. In some embodiments, the 5′-adapter and/or 3′-adapter comprise sequences complementary to oligonucleotides that can be amplified by a branched DNA (bDNA) process that may include signal or signal-generating oligonucleotide moieties.

In some instances, the methods, compositions, and kits disclosed herein comprise one or more adapters comprising one or more haptens. In some instances, the adapter comprises one or more haptens, wherein the one or more haptens comprise biotin or digoxigenin. In other instances, the haptens are selected from a list including, but not limited to: dinitrophenol (DNP), fluorescein, aniline, carboxyl derivatives of aniline (e.g., o-, m-, and p aminobenzoic acid), and urushiol. The 5′-adapter can further comprise one or more haptens. Alternatively, or additionally, the 3′-adapter further comprises one or more haptens. In some instances, the 5′-adapter and the 3′-adapter further comprise one or more haptens. In some instances, the 5′-adapter and the 3′-adapter comprise different haptens. For example, the 5′-adapter comprises a hapten comprising biotin and the 3′-adapter comprises a hapten comprising digoxigenin. In other instances, the 5′-adapter and the 3′-adapter comprise the same type of hapten. For example, both the 5′-adapter and the 3′-adapter comprise a hapten comprising biotin.

In other instances, the methods, compositions, and kits disclosed herein comprise one or more adapters comprising one or more signal moieties. For example, signal moieties include, but are not limited to, [5′-³²P]-labeled 5′-pNp-3′ (pNp); 5′-pN-3′-n-linker-detectable moiety; 5′-AppN-3′-n-linker-detectable moiety; and 5′-pNpN-n-linker-detectable moiety. The 5′-adapter can further comprise one or more signal moieties. Alternatively, or additionally, the 3′-adapter further comprises one or more signal moieties. In some instances, the 5′-adapter and the 3′-adapter further comprise one or more signal moieties. In some instances, the 5′-adapter and the 3′-adapter comprise different signal moieties. In other instances, the 5′-adapter and the 3′-adapter comprise the same type of signal moiety. Non-limiting examples of signal moieties include fluorescent species (e.g., fluorescein and rhodamine dyes and green fluorescent protein) and nanoparticles (e.g., nanogold as described in U.S. Pat. No. 7,824,863).

Alternatively, or additionally, the methods, compositions and kits disclosed herein comprise one or more adapters comprising one or more tags or probes. A non-limiting list of probes includes molecular probes such as Molecular Beacons, Scorpion probes and TaqMan probes. A non-limiting list of tags includes biotin and digoxigenin. In some instances, the tags or probes comprise sequences that can be used for sandwich hybridization. The 5′-adapter can further comprise one or more tags or probes. Alternatively, or additionally, the 3′-adapter further comprises one or more tags or probes. In some instances, the 5′-adapter and the 3′-adapter further comprise one or more tags or probes. In some instances, the 5′-adapter and the 3′-adapter comprise different tags or probes. In other instances, the 5′-adapter and the 3′-adapter comprise the same type of tag or probe.

The methods, compositions, and kits disclosed herein can further comprise one or more adapters further comprising a nucleotide linker sequence. A non-limiting list of linker sequences includes homopolynucleotide sequences such as (A)₄₀ (SEQ ID NO: 150) or repeats such as (ACA)₁₅ (SEQ ID NO: 151). The 5′-adapter can further comprise one or more linker sequences. Alternatively, or additionally, the 3′-adapter further comprises one or more linker sequences. In some instances, the 5′-adapter and the 3′-adapter further comprise one or more linker sequences. In some instances, the 5′-adapter and the 3′-adapter comprise different linker sequences. In other instances, the 5′-adapter and the 3′-adapter comprise the same type of linker sequence.

The haptens, signal moieties, tags, probes, and/or linker sequences disclosed herein can be located at the 5′ end of an adapter. For example, the haptens, signal moieties, tags, probes, and/or linker sequences disclosed herein are located at the 5′ end of a 5′-adapter. In another example, haptens, signal moieties, tags, probes, and/or linker sequences disclosed herein are located at the 5′ end of a 3′-adapter. Alternatively, or additionally, the haptens, signal moieties, tags, probes, and/or linker sequences disclosed herein are located at the 3′ end of an adapter. For example, the haptens, signal moieties, tags, probes, and/or linker sequences disclosed herein are located at the 3′ end of a 5′-adapter. In another example, haptens, signal moieties, tags, probes, and/or linker sequences disclosed herein are located at the 3′ end of a 3′-adapter. In some instances, the haptens, signal moieties, tags, probes, and/or linker sequences disclosed herein are located between the 5′ end and the 3′ end of an adapter. For example, the haptens, signal moieties, tags, probes, and/or linker sequences disclosed herein are located between the 5′ end and the 3′ end of a 5′-adapter. In another example, the haptens, signal moieties, tags, probes, and/or linker sequences disclosed herein are located between the 5′ end and the 3′ end of a 3′adapter.

The haptens, signal moieties, tags, probes, and/or linker sequences disclosed herein can be located within the sequence of an adapter. For example, the sequence at the 5′ end of a 3′-adapter can comprise a linker sequence. In another example, the sequence at the 3′ end of a 5′-adapter can comprise a probe sequence. In another example, the sequence in between the 3′ end and the 5′ end of an adapter sequence can comprise a linker sequence.

The haptens, signal moieties, tags, probes, and/or linker sequences disclosed herein can be attached to an adapter. For example, a hapten can be attached to the 5′ end of a 3′-adapter. In another example, a signal moiety can be attached to the 3′ end of a 5′-adapter. In another example, tag can be attached to the region between the 3′ end and the 5′ end of an adapter sequence.

In some embodiments, the disclosed adapter is a combo adapter (CAD). The combo adapter may comprise: a) nucleic acid residues, and, optionally, at least one modified nucleotide or non-nucleotide residue; b) a 5′-proximal segment and a 3′-proximal segment, wherein each proximal segment comprises at least one sequencing adapter, or primer binding site, or sequencing bar-code, or detection sequence, or a combination thereof; c) a 5′ end and a 3′ end that allow: i) intermolecular ligation of said combo adapter to a sample polynucleotide to produce an adapter-polynucleotide ligation product (also referred to as adapter-polynucleotide ligation product); and ii) circularization of the adapter-polynucleotide ligation product to produce a circularized adapter-polynucleotide ligation product.

In some embodiments, the disclosed adapter is a combo adapter (CAD). The combo adapter may comprise: a) nucleic acid residues, and, optionally, at least one modified nucleotide or non-nucleotide residue; b) a 5′-proximal segment and a 3′-proximal segment, wherein each proximal segment comprises at least one sequencing adapter; c) a 5′ end and a 3′ end that allow: i) intermolecular ligation of said combo adapter to a sample polynucleotide to produce an adapter-polynucleotide ligation product (also referred to as adapter-polynucleotide ligation product); and ii) circularization of the adapter-polynucleotide ligation product to produce a circularized adapter-polynucleotide ligation product.

In some embodiments, the disclosed adapter is a combo adapter (CAD). The combo adapter may comprise: a) nucleic acid residues, and, optionally, at least one modified nucleotide or non-nucleotide residue; b) a 5′-proximal segment and a 3′-proximal segment, wherein each proximal segment comprises at least one primer binding site; c) a 5′ end and a 3′ end that allow: i) intermolecular ligation of said combo adapter to a sample polynucleotide to produce an adapter-polynucleotide ligation product (also referred to as adapter-polynucleotide ligation product); and ii) circularization of the adapter-polynucleotide ligation product to produce a circularized adapter-polynucleotide ligation product.

In some embodiments, the disclosed adapter is a combo adapter (CAD). The combo adapter may comprise: a) nucleic acid residues, and, optionally, at least one modified nucleotide or non-nucleotide residue; b) a 5′-proximal segment and a 3′-proximal segment, wherein each proximal segment comprises at least sequencing bar-code; c) a 5′ end and a 3′ end that allow: i) intermolecular ligation of said combo adapter to a sample polynucleotide to produce an adapter-polynucleotide ligation product (also referred to as adapter-polynucleotide ligation product); and ii) circularization of the adapter-polynucleotide ligation product to produce a circularized adapter-polynucleotide ligation product.

In some embodiments, the disclosed adapter is a combo adapter (CAD). The combo adapter may comprise: a) nucleic acid residues, and, optionally, at least one modified nucleotide or non-nucleotide residue; b) a 5′-proximal segment and a 3′-proximal segment, wherein each proximal segment comprises at least one detection sequence; c) a 5′ end and a 3′ end that allow: i) intermolecular ligation of said combo adapter to a sample polynucleotide to produce an adapter-polynucleotide ligation product (also referred to as adapter-polynucleotide ligation product); and ii) circularization of the adapter-polynucleotide ligation product to produce a circularized adapter-polynucleotide ligation product.

In some embodiments, the combo adapter may be a 5′-adapter (also referred as 5′-CAD), which can be ligated to the 5′ end of the sample polynucleotide. In some embodiments, the combo adapter may be a 3′-adapter (also referred as 3′-CAD), which can be ligated to the 3′ end of the sample polynucleotide.

The CAD may comprise at least one sequence selected from: a sequencing adapter, a primer binding site, a detection sequence, a probe hybridization sequence, a capture oligonucleotide binding site, a polymerase binding site, an endonuclease restriction site, a sequencing bar-code, an indexing sequence, a Zip-code, one or more random nucleotides, a unique molecular identifier (UMI), sequencing flow-cell binding sites and combinations thereof. The 5′-proximal segment or the 3′-proximal segment of said combo adapter may comprise at least one sequencing adapter. The 5′-proximal segment and the 3′-proximal segment of said combo adapter may each comprise at least one sequencing adapter. The sequencing adapters may enable sequencing of the adapter-polynucleotide ligation product or complement thereof.

In some embodiments, the CAD comprises a template-deficient segment containing modified residues that can stop or inhibit a primer extension by a polymerase. The template-deficient segment may lie between the 5′-proximal segment and the 3′-proximal segments of the combo adapter. The template-deficient segment may lie between the 3′ proximal segment and the 5′ proximal segment of the combo adapter.

The template-deficient segment of CAD may contain at least one ribonucleotide (RNA), deoxyribonucleotide (DNA), or modified nucleic acid residue. Non-limiting examples of modified residues include a deoxyuridine (dU), an inosine (I), a deoxyinosine (dI), an Unlocked Nucleic Acid (UNA), a Locked Nucleic Acid (LNA) comprising a sugar modification, a Peptide Nucleic Acid (PNA), an abasic site, and a nucleic acid residue with a modification selected from: a 5-nitroindole base modification, a 2′-phosphate (2′-p), a 2′-NH₂, a 2′-NHR, a 2′-OMe, a 2′-O-alkyl, a 2′-F, a 2′-halo, a phosphorothioate (PS), and a disulfide (S—S) internucleotide bond modification.

In some embodiments, the 5′ proximal segment of the CAD comprises DNA. In some embodiments, the 5′ proximal segment comprises RNA. In some embodiments, the 5′ proximal segment of the CAD comprises a combination of RNA and DNA. In some embodiments, the 3′ proximal segment of the CAD comprises DNA. In some embodiments, the 3′ proximal segment of the CAD comprises RNA. In some embodiments, the 3′ proximal segment of the CAD comprises a combination of RNA and DNA. In some embodiments, the 3′ proximal segment of the CAD comprises one or more 2′-OMe modifications.

The CAD may comprise at least one cleavage (or cleavable) site(s). Said cleavage sites or cleavage sequences may be positioned within the proximal and 3′-proximal segments, or between its 5′-proximal and 3′-proximal segments of the combo adapter. In some embodiments, the cleavage site may be formed by internal secondary structure of the combo adapter. Said secondary structure may be stabilized by circularization of the combo adapter. In some embodiments, cleavage sites are substrates for nucleotide-specific or sequence-specific nucleases selected from: Uracil-DNA glycosylase (UDG), which cleaves at deoxyuridine (dU) residues; Endonuclease V, which cleaves DNA at deoxyinosine (dI) and RNA at inosine (i) residues; a restriction endonuclease, a ribozyme, a deoxyribozyme, artificial chemical nuclease, RNase H, RNase H II, Duplex-specific Nuclease, and Cas9 nuclease.

In some embodiments, the CAD template-deficient segment or the CAD cleavage at the cleavage site restricts rolling-circle amplification (RCA), but enables production of a monomeric nucleic acid (as opposed to multimeric products of RCA). Generally, the methods and compositions herein prevent/restrict rolling circle amplification. However, rolling circle amplification, as used herein, may be substituted with unrestricted primer extension by polymerase. In some embodiments, the template-deficient segment for primer extension by the polymerase inhibits RCA, but enables production of a monomeric nucleic acid. In some embodiments, the template-deficient segment for primer extension by the polymerase prohibits RCA, but enables production of a monomeric nucleic acid. In some embodiments, RCA does not occur at all.

In some embodiments, the methods disclosed herein comprise producing at least one monomeric nucleic acid that is specific to the target polynucleotide or portions thereof. By way of non-limiting example, the monomeric nucleic acid may comprise a sequence complementary to the sample polynucleotide, flanked by sequences that are complementary to at least a portion of the 5′-proximal segment and 3′-proximal segment of the CAD. In some embodiments, the monomeric nucleic acid may comprise a sequence corresponding to the target polynucleotide, flanked by sequences that correspond to at least a portion of the 5′-proximal segment and 3′-proximal segment of the CAD.

In some embodiments, the CAD comprises sequences selected from: primer binding; restriction sites, sequencing bar-code and indexing sequences, Zip-codes, at least one random nucleotide, and combination thereof.

In some embodiments, the CAD may comprise a probe binding site. The probe binding site or complement thereof may enable detection or purification of the polynucleotide-CAD ligation product and/or the circularized polynucleotide-CAD ligation product.

The 5′ end and/or 3′ end of the CAD may comprise a reversible blocking group. The reversible blocking group may prevent circularization and/or multimerization (or concatamerization) of the CAD during the first ligation step between the CAD and a polynucleotide. An activation (repair or unblocking) of said reversible blocking group by its conversion to an active (ligatable) group may allow circularization of the CAD-polynucleotide ligation product in the second ligation step. In some embodiments, said activation may be performed using an enzymatic, or chemical, or photochemical reaction, converting the reversible blocking groups to ligatable groups at the ends of the adapter. In some embodiments, the CAD comprises a 3′-end-blocking group. Non-limiting examples of 3′-end reversible blocking groups are: 3′-p, 2′-p, 2′,3′>p, 3′-O-(3-methoxyethyl) ether, and 3′-O-isovaleryl ester. In some embodiments, the CAD comprises a 5′-end-blocking group. Non-limiting examples of 5′-end reversible blocking groups are: 5′-ppp, 5′-5′-pp, 5′-p and 5′-OH. Non-limiting examples of active (ligatable) groups at the 5′ end are: 5′-App, 5′-p and 5′-OH. Non-limiting examples of active (ligatable) groups at the 3′ end are: 2′-OH/3′-OH, 2′-OH/3′-p and 2′,3′>p. A chemical group may be an active group or a reversible blocking group depending on the ligase used. For example, 3′-OH may be an active group for 3′-OH ligase and a blocking group for 5′-OH ligase; 3′-p may be an active group for 5′-OH ligase and a blocking group for 3′-OH ligase; 5′-OH may be an active group for 5′-OH ligase and a blocking group for 3′-OH ligase; and 5′-p or 5′-App may be an active group for 3′-OH ligase and a blocking group for 5′-OH ligase.

In some embodiments, the 3′-p, 2′-p and 2′,3′>p groups at the CAD 3′ end may be converted to 2′-OH/3′-OH by a polynucleotide kinase (PNK) either in the absence or presence of ATP. The 3′-p and 2′-p groups (but not 2′,3′>p) end groups may be converted to 2′-OH/3′-OH by an alkaline phosphatase. The said alkaline phosphatase may be selected from: Calf Intestinal phosphatase (CIP), Shrimp Alkaline Phosphatase (rSAP), APex™ Heat-labile alkaline phosphatase and Antarctic Phosphatase. In some embodiments, the 5′-OH group may be converted to 5′-p by polynucleotide kinase in the presence of ATP. The 5′-OH group at the CAD 5′-end may be converted to 5′-p in the presence of ATP by a polynucleotide kinase that also simultaneously removes 3′-p, 2′-p and 2′,3′>p. In some embodiments, the 5′-p group may be converted to 5′-OH without removal of 3′-p, 2′-p and 2′,3′>p (which groups in some cases may be required by a 5′-OH ligase) by a modified polynucleotide kinase derivative lacking 3′-end phosphatase activity in the absence of ATP and optional presence of ADP. In some embodiments, the 5′-ppp group may be converted to 5′-p by a pyrophosphatase or by RNA 5′ polyphosphatase.

In some embodiments, the CAD comprises at least one terminal residue that contains a reversible blocking group which requires chemical, photochemical or enzymatic modification to convert it into an active group prior to ligating and/or circularizing.

In some embodiments, the CAD is a 5′-CAD. The 5′-CAD may be ligated to the 5′ end of the sample polynucleotide. In some embodiments, the CAD comprises a nucleoside residue at its 3′ end selected from: uridine (U or rU), deoxyuridine (dU), deoxythymidine (dT), ribothymidine (rT), cytosine (C or rC), deoxycytosine (dC), adenosine (A or rA), deoxyadenosine (dA), guanosine (G or rG), deoxyguanosine (dG), inosine (I or rI), and deoxyinosine (dI). In some embodiments, 5′-CAD comprises 5′-OH and 3′-OH end groups wherein, after ligation of the CAD to the 5′-end of the sample polynucleotide, the 5′-OH group of the polynucleotide-CAD ligation product is converted to 5′-phosphate before the circularization step.

In some embodiments, the CAD is a 3′-CAD. In some embodiments, the 3′-CAD comprises a 5′-phosphate (5′-p) or 5′-adenylated (5′-App) group and a reversible 3′-end-blocking group that is converted into a 3′-OH group before circularizing. In some embodiments, the reversible 3′-end-blocking group is selected from: 3′-phosphate (3′-p), 2′,3′-cyclic phosphate (2′,3′>p), 3′-O-(α-methoxyethyl) ether, and 3′-O-isovaleryl ester.

### EXAMPLES

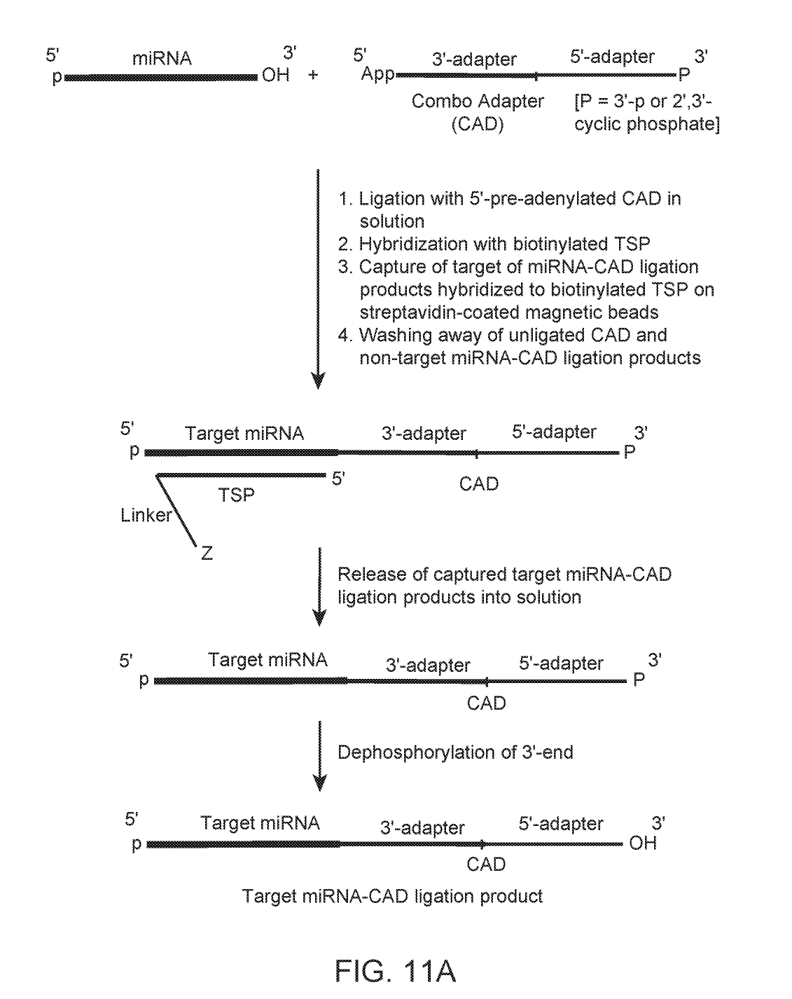

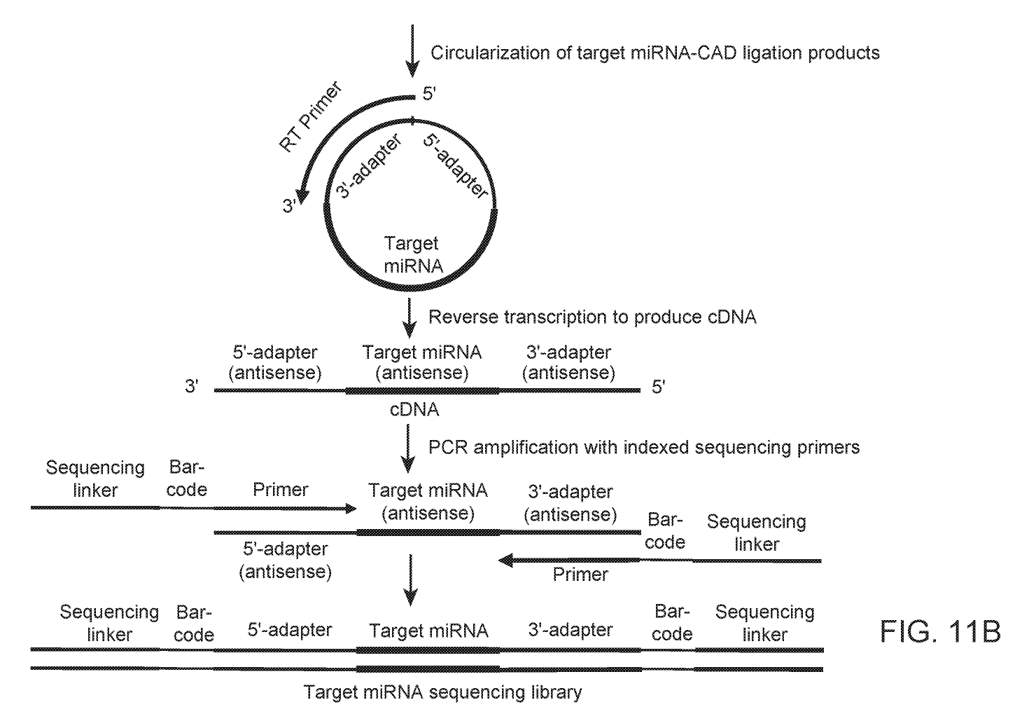

### Example 1. Targeted Sequencing of Selected miRNAs Isolated from Plasma Samples (FIG. 11A and 11B)

Total RNA was purified from 200 μl of each of 10 human plasma samples from healthy volunteers (Innovative Research) by using the miRNeasy Serum/Plasma kit (Qiagen) following the manufacturer's recommendations. RNA purified from each plasma sample was incubated with a pre-adenylated combo adapter (CAD), 5′-AppTGGAATTCTCGGGTGCCAAGG-idSp/idSp-r(GUUCAGAGUUCUACAGUCCGACGAUC)>p-3′ (DNA and RNA segments disclosed as SEQ ID NOS 1 and 152, respectively) (where App is 5′,5′-adenyl pyrophosphoryl group; >p is 2′,3′ cyclic phosphate; and idSp is a stretch of two abasic 1′,2′-dideoxyribose residues, dSpacers). CAD is comprised of segments of the standard TruSeq 3′-adapter, TGGAATTCTCGGGTGCCAAGG (SEQ ID. NO. 2) and 5′-adapter, GUUCAGAGUUCUACAGUCCGACGAUC (SEQ ID. NO. 3). The 10-μl ligation reactions contained 2.5 ng of CAD, 20 U/μl T4 RNA ligase 2 truncated K227Q-mutant (NEB), 1×T4 RNA ligase buffer (NEB), 10% PEG 8000, and 4 U/μl RNaseOUT (Invitrogen/ThermoFisher) and were incubated at 25° C. for 1 hour. Before adding to the ligation reaction mixture, both CAD and purified RNA were heated to 70° C. for 2 min and then cooled down (miRNAs were immediately placed on ice while CAD was cooled to 25° C. at the rate of 0.1° C./sec).

From a list of miRNAs previously found in plasma, 63 target miRNAs were selected (see Table 1) and biotinylated target-specific oligonucleotide probes (TSPs) were prepared that are substantially complementary to these target miRNAs and form duplexes with Tm ˜40° C. (TSP sequences are shown in Table 2). The TSPs were immobilized on magnetic beads and used to capture, concentrate and purify target miRNAs after their ligation to CAD. For this purpose, 80 μg of Streptavidin Magnetic Beads (NEB) were prepared by applying a magnet to the side of a tube containing the beads for approximately 30 sec, and the supernatant was removed. The beads were resuspended in 25 μl of a 10 μM (total concentration) mix of all 63 TSPs (0.16 μM each) in RNase-free water. The beads were washed twice with 100 μl of binding buffer (500 mM NaCl, 20 mM Tris-HCl pH 7.5, 1 mM EDTA), then resuspended in 50 μl of binding buffer and heated to 37° C. (Mix 1). The entire ligation reaction products were mixed with 50 μl of binding buffer and heated to 70° C. for 2 min (Mix 2), then combined with Mix 1 and further incubated at 25° C. for 10 minutes with occasional agitation. Then, the beads were washed four times to remove non-target RNA and unligated CAD molecules. The target miRNAs-CAD ligation products captured on the beads were eluted with 13.5 μl of pre-warmed (70° C.) RNase-free water.

To allow circularization of the eluted target miRNAs-CAD ligation products having miRNA 5′-p and CAD 2,3′-cyclic phosphate ends, the 3′ end of CAD was dephosphorylated by T4 polynucleotide kinase (PNK). The dephosphorylation by PNK and circularization by intramolecular ligation between the 5′-p and 3′-OH ends with T4 RNA ligase 1 (Rnl1) were run simultaneously in the same 22-μl reaction mixture containing target miRNA-CAD ligation products (13.5 μl), 1×T4 RNA ligase buffer, 7.5% PEG 8000, 10 U/μl PNK (NEB), 0.5 U/μl (NEB), and 2 U/μl RNaseOUT (Invitrogen/ThermoFisher) at 37° C. for 1 hour. The circularized miRNA-CAD products were then reverse transcribed in 40-μl RT reactions by incubating in the presence of 400 Units of SuperScript IV reverse transcriptase (Invitrogen/ThermoFisher), 500 μM dNTPs, 2.5 mM DTT, 1×SSIV buffer and 1.25 μM RT primer, CCTTGGCACCCGAGAATTCCA (SEQ ID NO. 130), at 50° C. for 30 minutes and then at 80° C. for 10 minutes. The RT primer has the same sequence as the TruSeq RTP-1 primer (Illumina) but with 1 nt deleted from its 5′ end. The RT reaction stops at the abasic site of the CAD, which prevents rolling-circle amplification and results in synthesis of nearly uniformly-sized cDNA products as shown in FIG. 11A and 11B.

The cDNA products of reverse transcription were then amplified by PCR to generate sequencing libraries of the target miRNAs. The PCR reactions were performed in the presence of 0.1 U/μl of LongAmp Taq DNA Polymerase (NEB), 1×LongAmp Taq Reaction Buffer, and 300 μM dNTPs using pairs of standard TruSeq PCR primers at 0.7 μM each: a universal forward primer RP1 (AATGATACGGCGACCACCGAGATCTACACGTTCAGAGTTCTACAGTCCG) (SEQ ID NO. 131) and different reverse, indexed RPI primers (see Table 3) to distinguish the sequencing libraries prepared for RNA isolated from different plasma samples.

The target miRNA sequencing libraries prepared with the different indexed primers were mixed and sequenced simultaneously on a MiSeq instrument (Illumina). Sequencing reads were trimmed of adaptor sequences by using Cutadapt (Martin, M. et al. 2011. EMBnet.journal 17: 10-12) and trimmed reads were aligned to a custom miRNA reference file using Bowtie2 (Langmead, B., Salzberg, S. L. 2012. Nat. Methods 9: 357-9). Reads mapping to miRNAs were counted using a custom script. Despite very low concentrations of miRNAs in plasma and significant variation of miRNA levels among different plasma samples, we were able to reliably quantify (with 10 or more sequencing reads per million) 61 miRNAs in each of the 10 tested plasma samples out of the selected 63 miRNAs.

TABLE 1
List of the selected (target) miRNAs

| | miRNA sequence (5′ to 3′) | |
|---|---|---|
| | (Note: all miRNAs are | |
| | phosphorylated at their | SEQ |
| miRNA name | 5′ end (5′-p) | ID NO. |
| hsa-let-7a-5p | UGAGGUAGUAGGUUGUAUAGUU | 4 |
| hsa-miR-100-5p | AACCCGUAGAUCCGAACUUGUG | 5 |
| hsa-miR-101-3p | UACAGUACUGUGAUAACUGAA | 6 |
| hsa-miR-103a-3p | AGCAGCAUUGUACAGGGCUAUGA | 7 |
| hsa-miR-106b-3p | CCGCACUGUGGGUACUUGCUGC | 8 |
| hsa-miR-107 | AGCAGCAUUGUACAGGGCUAUCA | 9 |
| hsa-miR-10b-5p | UACCCUGUAGAACCGAAUUUGUG | 10 |
| hsa-miR-122-5p | UGGAGUGUGACAAUGGUGUUUG | 11 |
| hsa-miR-125a-5p | UCCCUGAGACCCUUUAACCUGUGA | 12 |
| hsa-miR-125b-2-3p | UCACAAGUCAGGCUCUUGGGAC | 13 |
| hsa-miR-125b-5p | UCCCUGAGACCCUAACUUGUGA | 14 |
| hsa-miR-127-3p | UCGGAUCCGUCUGAGCUUGGCU | 15 |
| hsa-miR-1298-5p | UUCAUUCGGCUGUCCAGAUGUA | 16 |
| hsa-miR-1307-3p | ACUCGGCGUGGCGUCGGUCGUG | 17 |
| hsa-miR-140-3p | UACCACAGGGUAGAACCACGG | 18 |
| hsa-miR-141-3p | UAACACUGUCUGGUAAAGAUGG | 19 |
| hsa-miR-143-3p | UGAGAUGAAGCACUGUAGCUC | 20 |
| hsa-miR-145-5p | GUCCAGUUUUCCCAGGAAUCCCU | 21 |
| hsa-miR-146a-5p | UGAGAACUGAAUUCCAUGGGUU | 22 |
| hsa-miR-148a-3p | UCAGUGCACUACAGAACUUUGU | 23 |
| hsa-miR-151a-3p | CUAGACUGAAGCUCCUUGAGG | 24 |
| hsa-miR-16-5p | UAGCAGCACGUAAAUAUUGGCG | 25 |
| hsa-miR-17-5p | CAAAGUGCUUACAGUGCAGGUAG | 26 |
| hsa-miR-181a-5p | AACAUUCAACGCUGUCGGUGAGU | 27 |
| hsa-miR-182-5p | UUUGGCAAUGGUAGAACUCACACU | 28 |
| hsa-miR-186-5p | CAAAGAAUUCUCCUUUUGGGCU | 29 |
| hsa-miR-191-5p | CAACGGAAUCCCAAAAGCAGCUG | 30 |
| hsa-miR-192-5p | CUGACCUAUGAAUUGACAGCC | 31 |
| hsa-miR-19a-3p | UGUGCAAAUCUAUGCAAAACUGA | 32 |
| hsa-miR-204-5p | UUCCCUUUGUCAUCCUAUGCCU | 33 |
| hsa-miR-210-3p | CUGUGCGUGUGACAGCGGCUGA | 34 |
| hsa-miR-21-3p | CAACACCAGUCGAUGGGCUGU | 35 |
| hsa-miR-215-5p | AUGACCUAUGAAUUGACAGAC | 36 |
| hsa-miR-21-5p | UAGCUUAUCAGACUGAUGUUGA | 37 |
| hsa-miR-221-3p | AGCUACAUUGUCUGCUGGGUUUC | 38 |
| hsa-miR-22-3p | AAGCUGCCAGUUGAAGAACUGU | 39 |
| hsa-miR-24-3p | UGGCUCAGUUCAGCAGGAACAG | 40 |
| hsa-miR-25-3p | CAUUGCACUUGUCUCGGUCUGA | 41 |
| hsa-miR-26a-5p | UUCAAGUAAUCCAGGAUAGGCU | 42 |
| hsa-miR-27a-3p | UUCACAGUGGCUAAGUUCCGC | 43 |
| hsa-miR-28-3p | CACUAGAUUGUGAGCUCCUGGA | 44 |
| hsa-miR-30a-5p | UGUAAACAUCCUCGACUGGAAG | 45 |
| hsa-miR-31-5p | AGGCAAGAUGCUGGCAUAGCU | 46 |
| hsa-miR-320a | AAAAGCUGGGUUGAGAGGGCGA | 47 |
| hsa-miR-345-5p | GCUGACUCCUAGUCCAGGGCUC | 48 |
| hsa-miR-34c-5p | AGGCAGUGUAGUUAGCUGAUUGC | 49 |
| hsa-miR-363-3p | AAUUGCACGGUAUCCAUCUGUA | 50 |
| hsa-miR-375 | UUUGUUCGUUCGGCUCGCGUGA | 51 |
| hsa-miR-378a-3p | ACUGGACUUGGAGUCAGAAGGC | 52 |
| hsa-miR-423-3p | AGCUCGGUCUGAGGCCCCUCAGU | 53 |
| hsa-miR-423-5p | UGAGGGGCAGAGAGCGAGACUUU | 54 |
| hsa-miR-425-5p | AAUGACACGAUCACUCCCGUUGA | 55 |
| hsa-miR-451a | AAACCGUUACCAUUACUGAGUU | 56 |
| hsa-miR-483-5p | AAGACGGGAGGAAAGAAGGGAG | 57 |
| hsa-miR-486-3p | CGGGGCAGCUCAGUACAGGAU | 58 |
| hsa-miR-486-5p | UCCUGUACUGAGCUGCCCCGAG | 59 |
| hsa-miR-501-3p | AAUGCACCCGGGCAAGGAUUCU | 60 |
| hsa-miR-520d-5p | CUACAAAGGGAAGCCCUUUC | 61 |
| hsa-miR-524-5p | CUACAAAGGGAAGCACUUUCUC | 62 |
| hsa-miR-769-5p | UGAGACCUCUGGGUUCUGAGCU | 63 |
| hsa-miR-92a-3p | UAUUGCACUUGUCCCGGCCUGU | 64 |
| hsa-miR-93-5p | CAAAGUGCUGUUCGUGCAGGUAG | 65 |
| hsa-miR-99a-5p | AACCCGUAGAUCCGAUCUUGUG | 66 |

TABLE 2
Target-specific oligonucleotide probes (TSPs)

| | TSP sequence (5′ to 3′) | |
|---|---|---|
| | (Note: All TSPs are | SEQ |
| | biotinylated at 3′-end | ID |
| TSP name | via a /3BioTEG/ linker | NO. |
| TSP_hsa-let-7a-5p | CTATACAACCTACTACC/3BioTEG/ | 67 |
| TSP_hsa-miR-100-5p | AAGTTCGGATCTACG/3BioTEG/ | 68 |
| TSP_hsa-miR-101-3p | CAGTTATCACAGTACTG/3BioTEG/ | 69 |
| TSP_hsa-miR-103a-3p | ATAGCCCTGTACAAT/3BioTEG/ | 70 |
| TSP_hsa-miR-106b-3p | CAAGTACCCACAGTG/3BioTEG/ | 71 |
| TSP_hsa-miR-107 | TAGCCCTGTACAATG/3BioTEG/ | 72 |
| TSP_hsa-miR-10b-5p | CAAATTCGGTTCTACA/3BioTEG/ | 73 |
| TSP_hsa-miR-122-5p | AACACCATTGTCACA/3BioTEG/ | 74 |
| TSP_hsa-miR-125a-5p | ACAGGTTAAAGGGTC/3BioTEG/ | 75 |
| TSP_hsa-miR-125b-2-3p | CAAGAGCCTGACTTG/3BioTEG/ | 76 |
| TSP_hsa-miR-125b-5p | CAAGTTAGGGTCTCA/3BioTEG/ | 77 |
| TSP_hsa-miR-127-3p | AAGCTCAGACGGAT/3BioTEG/ | 78 |
| TSP_hsa-miR-1298-5p | TGGACAGCCGAAT/3BioTEG/ | 79 |
| TSP_hsa-miR-1307-3p | ACCGACGCCAC/3BioTEG/ | 80 |
| TSP_hsa-miR-140-3p | GTGGTTCTACCCTGT/3BioTEG/ | 81 |
| TSP_hsa-miR-141-3p | TCTTTACCAGACAGTG/3BioTEG/ | 82 |
| TSP_hsa-miR-143-3p | GCTACAGTGCTTCAT/3BioTEG/ | 83 |
| TSP_hsa-miR-145-5p | TTCCTGGGAAAAC/3BioTEG/ | 84 |
| TSP_hsa-miR-146a-5p | CCCATGGAATTCAGT/3BioTEG/ | 85 |
| TSP_hsa-miR-148a-3p | AGTTCTGTAGTGCAC/3BioTEG/ | 86 |
| TSP_hsa-miR-151a-3p | AAGGAGCTTCAGTCT/3BioTEG/ | 87 |
| TSP_hsa-miR-16-5p | CAATATTTACGTGCT/3BioTEG/ | 88 |
| TSP_hsa-miR-17-5p | CTGCACTGTAAGCA/3BioTEG/ | 89 |
| TSP_hsa-miR-181a-5p | CGACAGCGTTGAA/3BioTEG/ | 90 |
| TSP_hsa-miR-182-5p | GTGAGTTCTACCATTG/3BioTEG/ | 91 |
| TSP_hsa-miR-186-5p | CCCAAAAGGAGAATTC/3BioTEG/ | 92 |
| TSP_hsa-miR-191-5p | GCTTTTGGGATTC/3BioTEG/ | 93 |
| TSP_hsa-miR-192-5p | CTGTCAATTCATAGGTC/3BioTEG/ | 94 |
| TSP_hsa-miR-19a-3p | AGTTTTGCATAGATTTG/3BioTEG/ | 95 |
| TSP_hsa-miR-204-5p | GCATAGGATGACAAAG/3BioTEG/ | 96 |
| TSP_hsa-miR-210-3p | CGCTGTCACACG/3BioTEG/ | 97 |
| TSP_hsa-miR-21-3p | CCCATCGACTGGT/3BioTEG/ | 98 |
| TSP_hsa-miR-215-5p | CTGTCAATTCATAGGTC/3BioTEG/ | 99 |
| TSP_hsa-miR-21-5p | CATCAGTCTGATAAGC/3BioTEG/ | 100 |
| TSP_hsa-miR-221-3p | CCAGCAGACAATGT/3BioTEG/ | 101 |
| TSP_hsa-miR-22-3p | AGTTCTTCAACTGGC/3BioTEG/ | 102 |
| TSP_hsa-miR-24-3p | TCCTGCTGAACTGA/3BioTEG/ | 103 |
| TSP_hsa-miR-25-3p | AGACCGAGACAAGT/3BioTEG/ | 104 |
| TSP_hsa-miR-26a-5p | TATCCTGGATTACTTG/3BioTEG/ | 105 |
| TSP_hsa-miR-27a-3p | GAACTTAGCCACTGT/3BioTEG/ | 106 |
| TSP_hsa-miR-28-3p | AGGAGCTCACAATCT/3BioTEG/ | 107 |
| TSP_hsa-miR-30a-5p | CAGTCGAGGATGTTT/3BioTEG/ | 108 |
| TSP_hsa-miR-31-5p | ATGCCAGCATCTT/3BioTEG/ | 109 |
| TSP_hsa-miR-320a | CCTCTCAACCCAG/3BioTEG/ | 110 |
| TSP_hsa-miR-345-5p | CCCTGGACTAGGAG/3BioTEG/ | 111 |
| TSP_hsa-miR-34c-5p | ATCAGCTAACTACACT/3BioTEG/ | 112 |
| TSP_hsa-miR-363-3p | CAGATGGATACCGTG/3BioTEG/ | 113 |
| TSP_hsa-miR-375 | GAGCCGAACGAAC/3BioTEG/ | 114 |
| TSP_hsa-miR-378a-3p | TTCTGACTCCAAGTC/3BioTEG/ | 115 |
| TSP_hsa-miR-423-3p | GGGCCTCAGACC/3BioTEG/ | 116 |
| TSP_hsa-miR-423-5p | AGTCTCGCTCTCTG/3BioTEG/ | 117 |
| TSP_hsa-miR-425-5p | CGGGAGTGATCGT/3BioTEG/ | 118 |
| TSP_hsa-miR-451a | CAGTAATGGTAACGG/3BioTEG/ | 119 |
| TSP_hsa-miR-483-5p | TTCTTTCCTCCCGT/3BioTEG/ | 120 |
| TSP_hsa-miR-486-3p | CTGTACTGAGCTGC/3BioTEG/ | 121 |
| TSP_hsa-miR-486-5p | GGGCAGCTCAGTA/3BioTEG/ | 122 |
| TSP_hsa-miR-501-3p | AATCCTTGCCCGG/3BioTEG/ | 123 |
| TSP_hsa-miR-520d-5p | GGCTTCCCTTTG/3BioTEG/ | 124 |
| TSP_hsa-miR-524-5p | AAGTGCTTCCCTT/3BioTEG/ | 125 |
| TSP_hsa-miR-769-5p | AGAACCCAGAGGTC/3BioTEG/ | 126 |
| TSP_hsa-miR-92a-3p | GGGACAAGTGCAA/3BioTEG/ | 127 |
| TSP_hsa-miR-93-5p | CCTGCACGAACAG/3BioTEG/ | 128 |
| TSP_hsa-miR-99a-5p | AAGATCGGATCTACG/3BioTEG/ | 129 |

TABLE 3
Indexed sequencing primers

| Primer | | SEQ |
|---|---|---|
| name | Primer sequence (5′ to 3′) | ID NO. |
| RPI34 | CAAGCAGAAGACGGCATACGAGATGCCA | 132 |
| | TGGTGACTGGAGTTCCTTGGCACCCGAGA | |
| | ATTCC*A | |
| RPI35 | CAAGCAGAAGACGGCATACGAGATAAAA | 133 |
| | TGGTGACTGGAGTTCCTTGGCACCCGAGA | |
| | ATTCC*A | |
| RPI36 | CAAGCAGAAGACGGCATACGAGATTGTT | 134 |
| | GGGTGACTGGAGTTCCTTGGCACCCGAGA | |
| | ATTCC*A | |
| RPI37 | CAAGCAGAAGACGGCATACGAGATATTC | 135 |
| | CGGTGACTGGAGTTCCTTGGCACCCGAGA | |
| | ATTCC*A | |
| RPI38 | CAAGCAGAAGACGGCATACGAGATAGCT | 136 |
| | AGGTGACTGGAGTTCCTTGGCACCCGAGA | |
| | ATTCC*A | |
| RPI39 | CAAGCAGAAGACGGCATACGAGATGTAT | 137 |
| | AGGTGACTGGAGTTCCTTGGCACCCGAGA | |
| | ATTCC*A | |
| RPI40 | CAAGCAGAAGACGGCATACGAGATTCTG | 138 |
| | AGGTGACTGGAGTTCCTTGGCACCCGAGA | |
| | ATTCC*A | |
| RPI41 | CAAGCAGAAGACGGCATACGAGATGTCG | 139 |
| | TCGTGACTGGAGTTCCTTGGCACCCGAGA | |
| | ATTCC*A | |
| RPI42 | CAAGCAGAAGACGGCATACGAGATCGAT | 140 |
| | TAGTGACTGGAGTTCCTTGGCACCCGAGA | |
| | ATTCC*A | |
| RPI43 | CAAGCAGAAGACGGCATACGAGATGCTG | 141 |
| | TAGTGACTGGAGTTCCTTGGCACCCGAGA | |
| | ATTCC*A | |

Note:
* = Phosphorothioate bond

The following Examples 2 through 5 describe (in general) capture of target RNAs and their products of processing at different Steps by hybridization with the TSP. Steps described in these examples may be combined in a method disclosed herein.

### Example 2. Capture of Target Polynucleotides from a Pool of Sample Polynucleotides

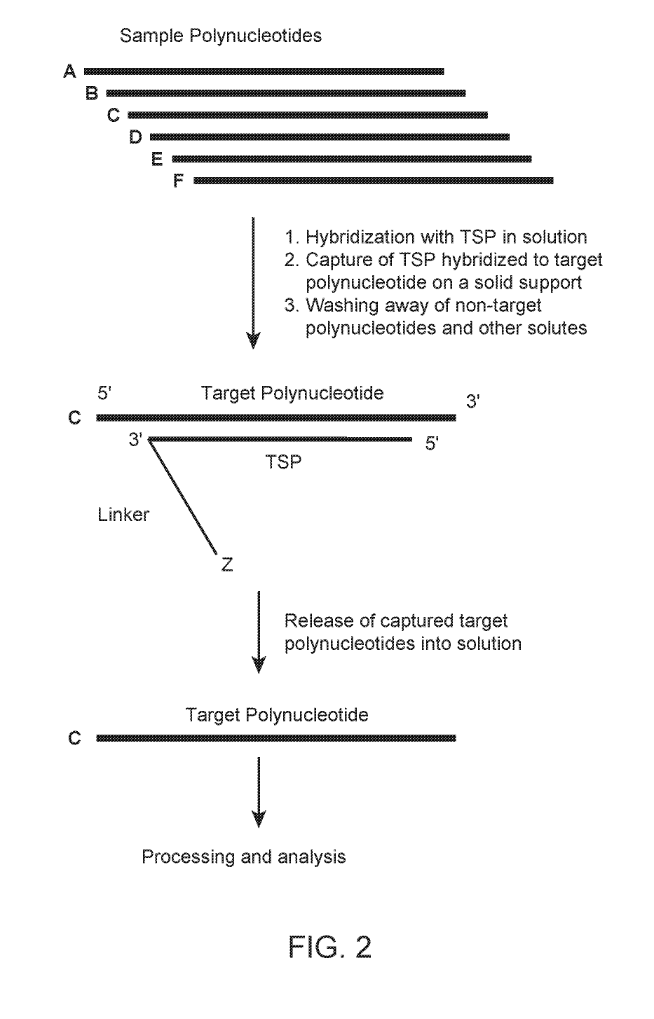

Single-stranded (or denatured double-stranded) RNA and/or DNA polynucleotides are hybridized with TSPs that are specific to target polynucleotides. The number of target polynucleotides (and target-specific probes) may vary from one to several thousand. Capture of TSP-polynucleotide hybrids on a solid support (e.g., magnetic beads) allows concentration of the target polynucleotides from dilute samples and/or washing away of non-target polynucleotides and other solutes, including inhibitors of certain enzymatic reactions that may be present in the samples. The concentrated and purified target polynucleotides are then released into solution for further procedures such as ligation of adapter(s), circularization, and analysis. (See, e.g., FIG. 2).

### Example. 3. Sequential Ligation of 3′-Adapter and 5′-Adapter to the Ends of Sample Polynucleotides and Capture of Target Polynucleotide-Adapter Ligation Products

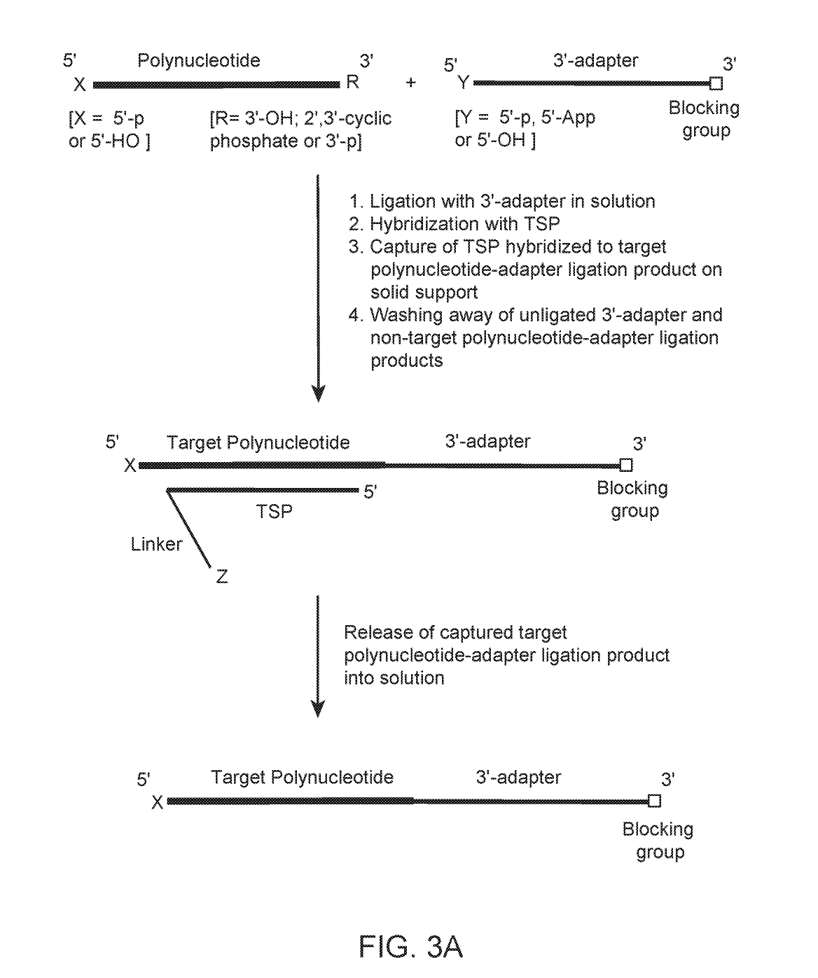

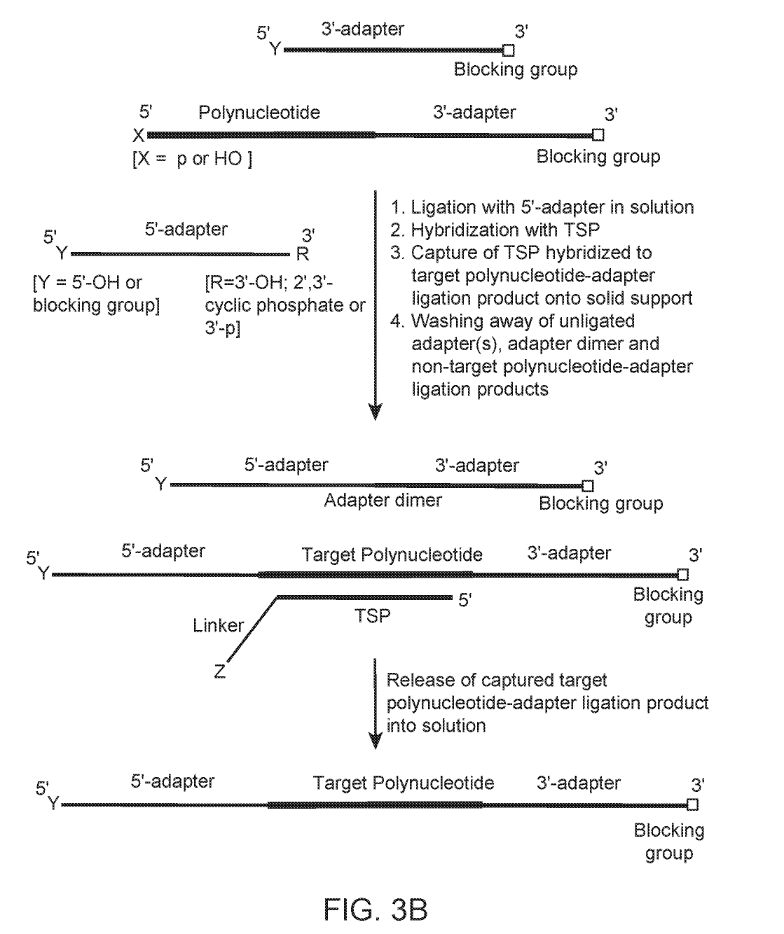

Target polynucleotides ligated to 3′-adapter (FIG. 3A) 5′-adapter (FIG. 4A) are captured to separate the ligation product from the unligated adapter and avoid the formation of adapter dimers in the subsequent adapter ligation step. Alternatively, capture of target polynucleotides ligated to both 3′-adapter and 5′-adapter (FIG. 3B and FIGS. 4B, 4C and 4D), and separation of the ligation products from the unligated adapter(s) as well as adapter dimers is performed. In contrast to FIG. 4 B, wherein 3′-adapter ligating is performed via intermolecular (splint-independent) reaction, the ligating of 3′-adapter in FIGS. 4C-D is performed via splint-dependent (or template-dependent) reactions, wherein TSP serves as both as splint (or template) and capture probes. In FIG. 4C, the TSP is complementary to a 3′-end proximal segment of the target polynucleotide and to a 5′-end proximal segment of the 3′-adapter, thereby aligning these ends head-to-tail within the duplex formed with the splint. In FIG. 4D, the TSP comprises: (i) a 3′-end proximal segment, which is complementary to a 3-end segment of the target polynucleotide; (ii) a 5′-end proximal segment, which is complementary to a 5′-end proximal segment of the 3′ adapter; and (iii) a linker connecting the 3′-end proximal segment and the 5′-segment of the TSP, wherein the linker is not complementary to one or more nucleotide(s) at the polynucleotide's 3′ end and at the 3′-adapter's 5′ end.

### Example 4. Capture of Target-Specific cDNAs (Complementary DNAs) After Reverse Transcription of Polynucleotide-Adapter Ligation Products

Polynucleotides with a ligated 5′-adapter comprising RNA nucleotides and a 3′-adapter comprising either DNA (FIG. 5A) or RNA nucleotides (FIG. 5B) is subjected to reverse transcription. After reverse transcription and degradation of RNA templates (e.g. by RNase H), the cDNAs comprising antisense sequences of target polynucleotides are captured and separated from cDNA products from non-target polynucleotides and adapter dimers.

### Example 5. Capture of Target-Specific cDNAs after RT-PCR or PCR Amplification of Polynucleotide-Adapter Ligation Products

The reverse transcription and optional degradation of RNA templates may be required if target polynucleotides and/or one or both adapters comprise RNA nucleotides. PCR amplification of polynucleotides ligated with two (5′- and 3′-) adapters in the presence of an excess of one of the primers generates single-stranded amplicons that are captured and separated from the amplification products related to non-target polynucleotides and adapter dimers.

### Example 6. Preparation of Strand-Specific Sequencing Libraries from cDNAs Comprising Sequences of 5′-Adapter, Target Polynucleotides and 3′-Adapter

Adapters comprising sequences that are compatible with PCR primers specific for the NGS method are used for sequencing FIG. 6.

### Example 7. Ligation of a Single Combo Adapter (CAD) to the Ends of Sample Polynucleotides and Capture of Target Polynucleotide-CAD Ligation Products

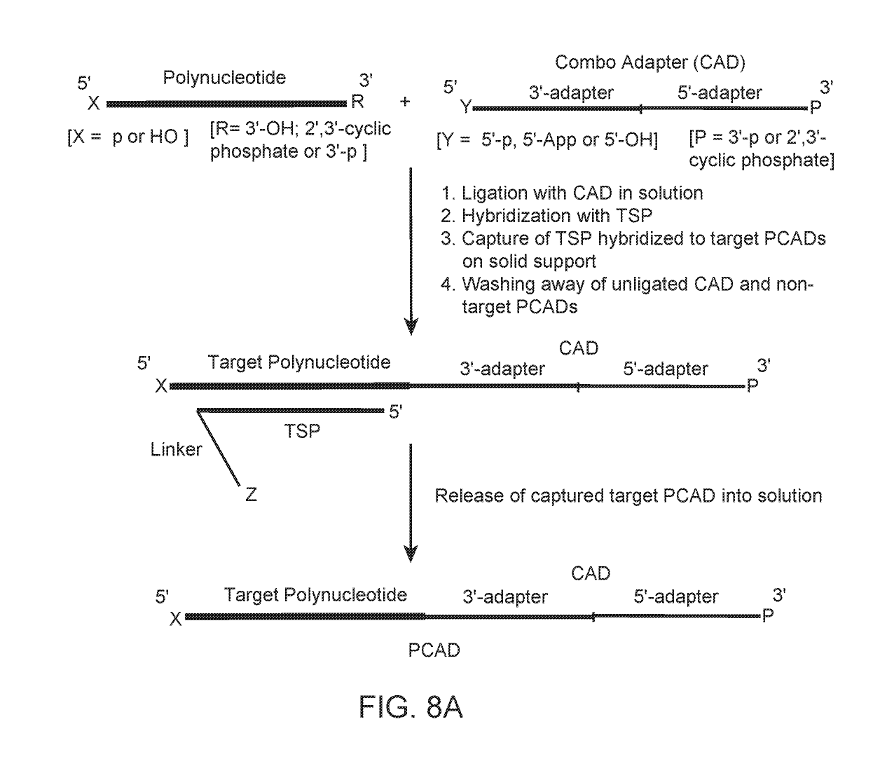

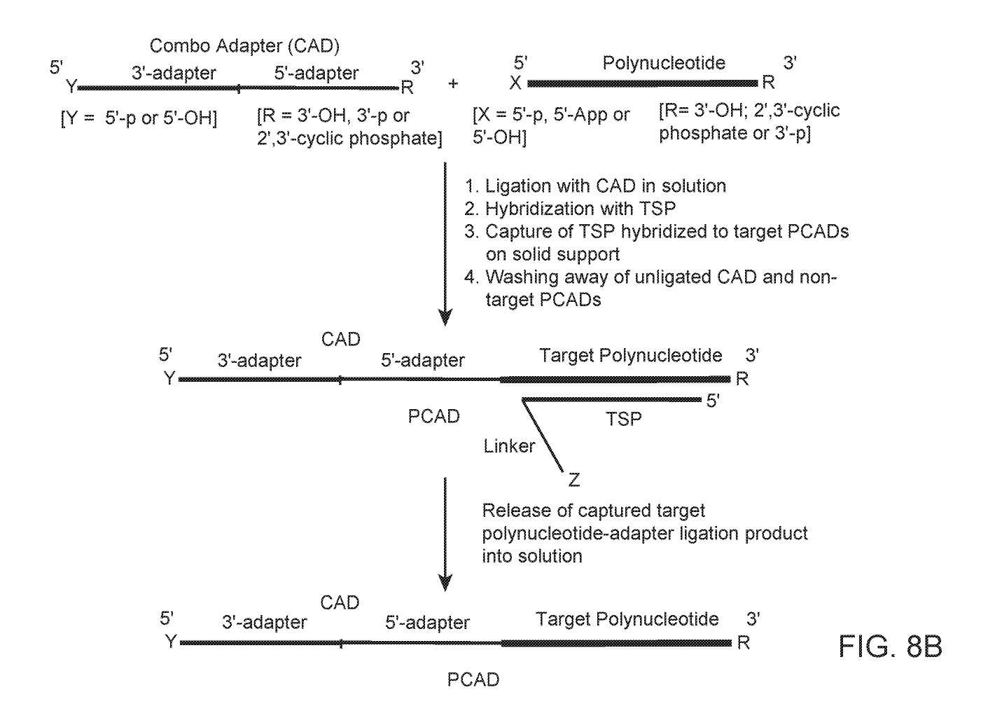

A CAD comprising sequences of a 3′-adapter and 5′-adapter presented in FIGS. 3-7, but in opposite order from that of the adapter dimer (compare with FIG. 4B) is used. Optionally, these 3′- and 5′-adapter sequences within the CAD can be separated by one or more template-deficient modifications that stop primer extension by a polymerase. The CAD can be ligated either to the 3′-end (FIG. 8A) or 5′-end (FIG. 8B) of the polynucleotide to form polynucleotide-CAD ligation products (PCADs). Different combinations of terminal groups at the polynucleotide and CAD ends allow different enzymatic ligation steps. Some terminal groups also can serve as reversible blocking groups to prevent circularization (and multimerization) of the polynucleotide and/or CAD that may compete with ligation of polynucleotide with CAD. Capture of target polynucleotides ligated to the CAD allows separation of the PCADs from the unligated CAD.

### Example 8. Circularization of Polynucleotide-CAD Ligation Products and Capture of Circularized Target Polynucleotide-CAD Ligation Products

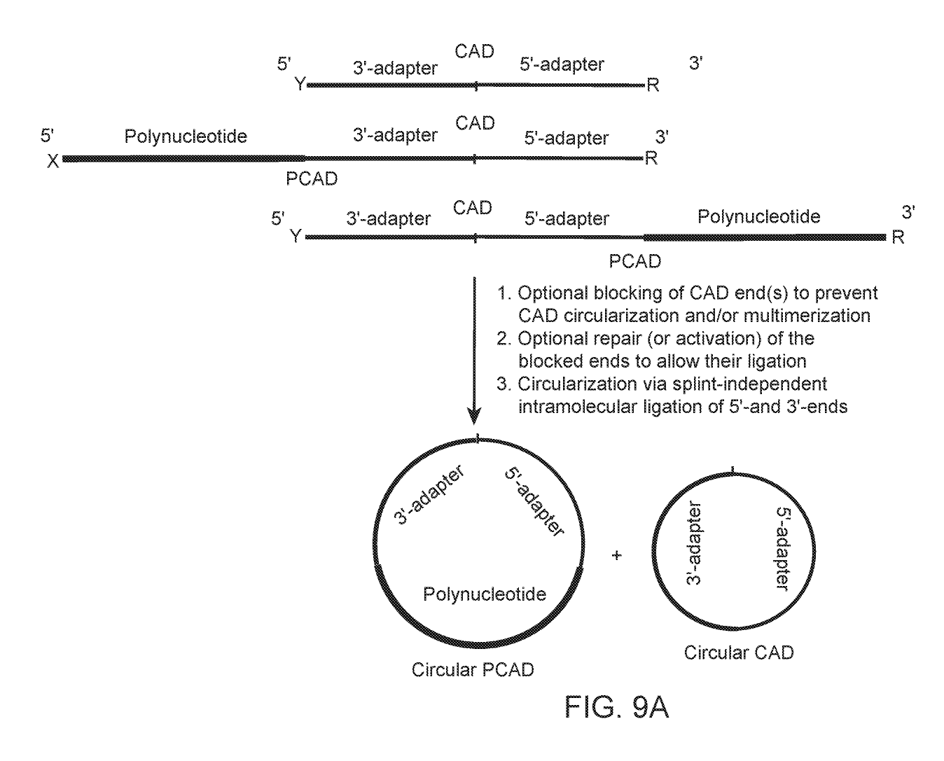

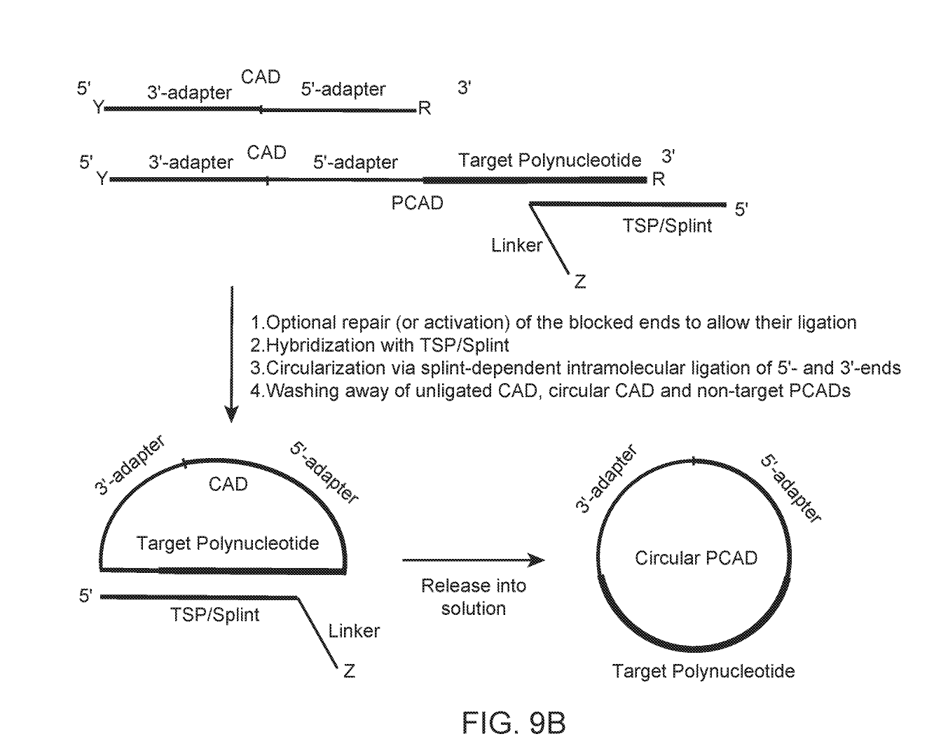

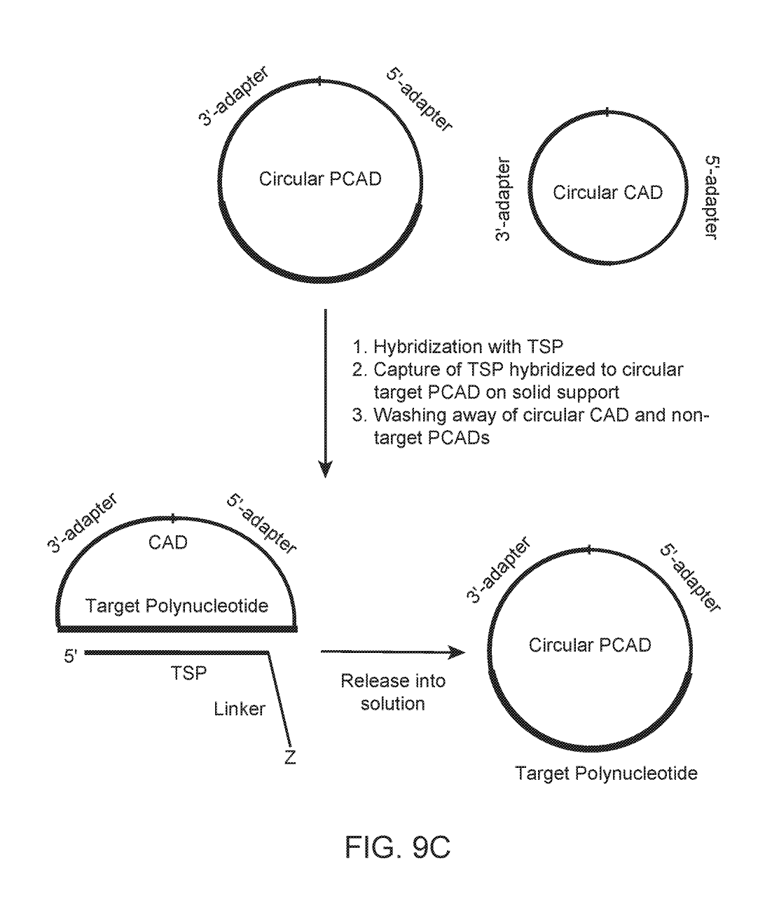

Circularization (FIG. 9A) of the polynucleotide-CAD ligation products (PCADs) and unligated CAD creates templates with the same order of 5′- and 3′-adapters relative to polynucleotide insert as the two-adapter ligation approach (see FIG. 3B). To allow the circularization of the PCADs, the reversible blocking groups at the available ends of polynucleotide and CAD segments is repaired (e.g., by phosphorylation or de-phosphorylation). Such repair also may allow circularization and multimerization of CADs that may be present in access relative to polynucleotide-CAD ligation products. To prevent the circularization of unligated CAD, the CAD end that participates in ligation to the polynucleotide can be enzymatically or chemically blocked. The circularized polynucleotide-CAD ligation products can be captured and purified from circular non-target polynucleotide-CAD ligation products and circular CAD (FIG. 9B) similar to their linear counterparts (see FIG. 3B).

### Example 9. Capture of Target-Specific cDNAs after Reverse Transcription of the Circular Polynucleotide-Combo Adapter Ligation Products (PCADs)

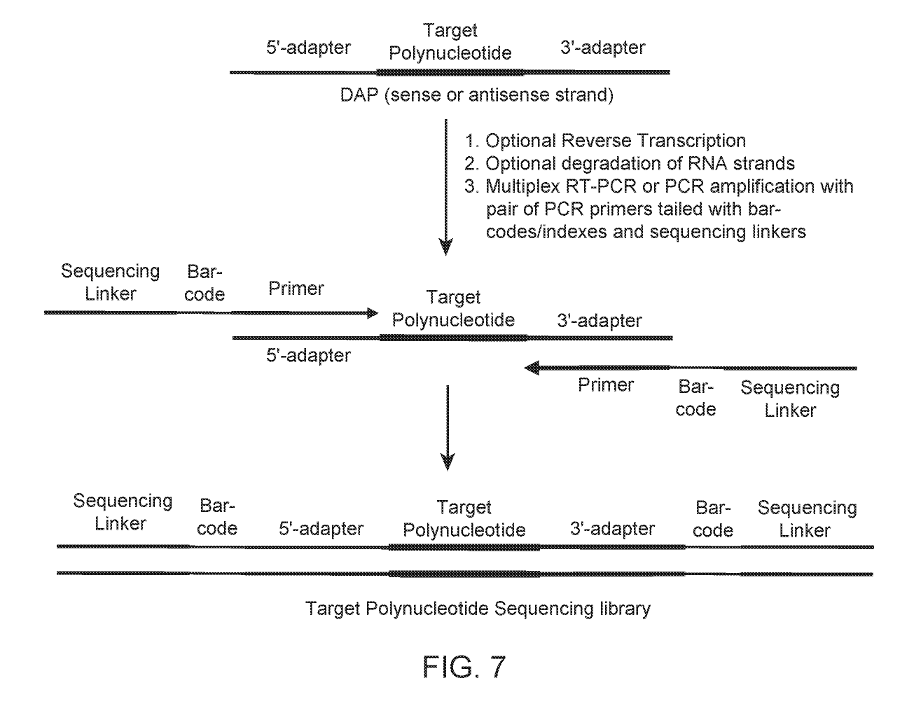

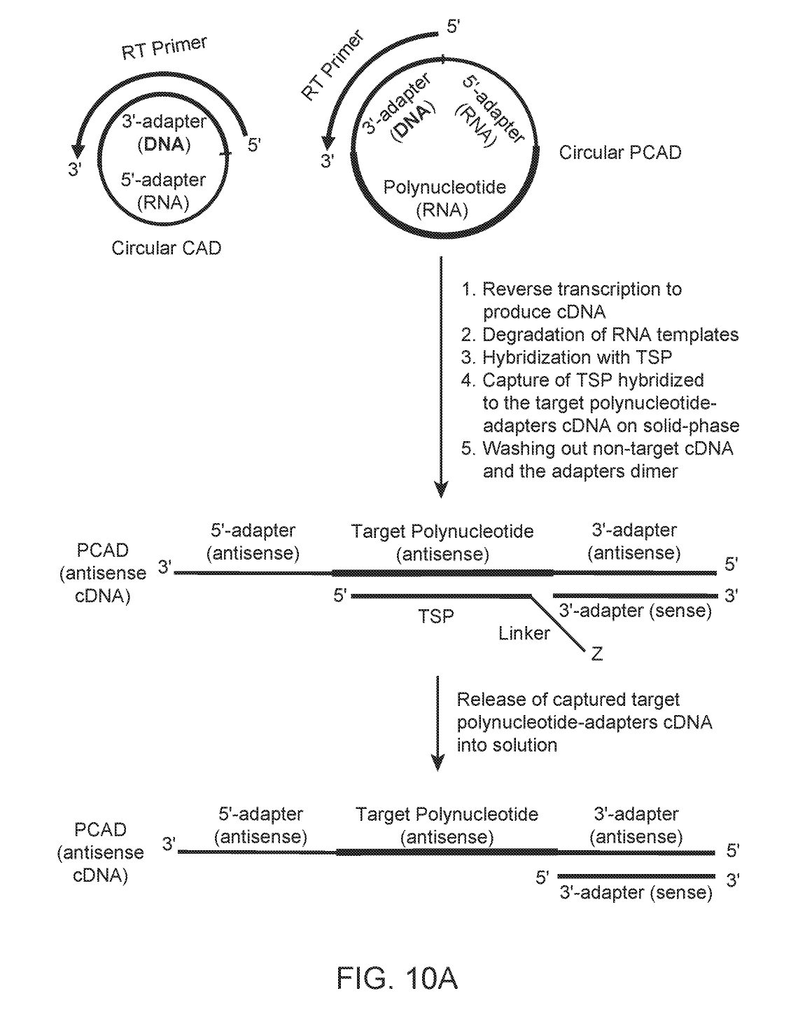

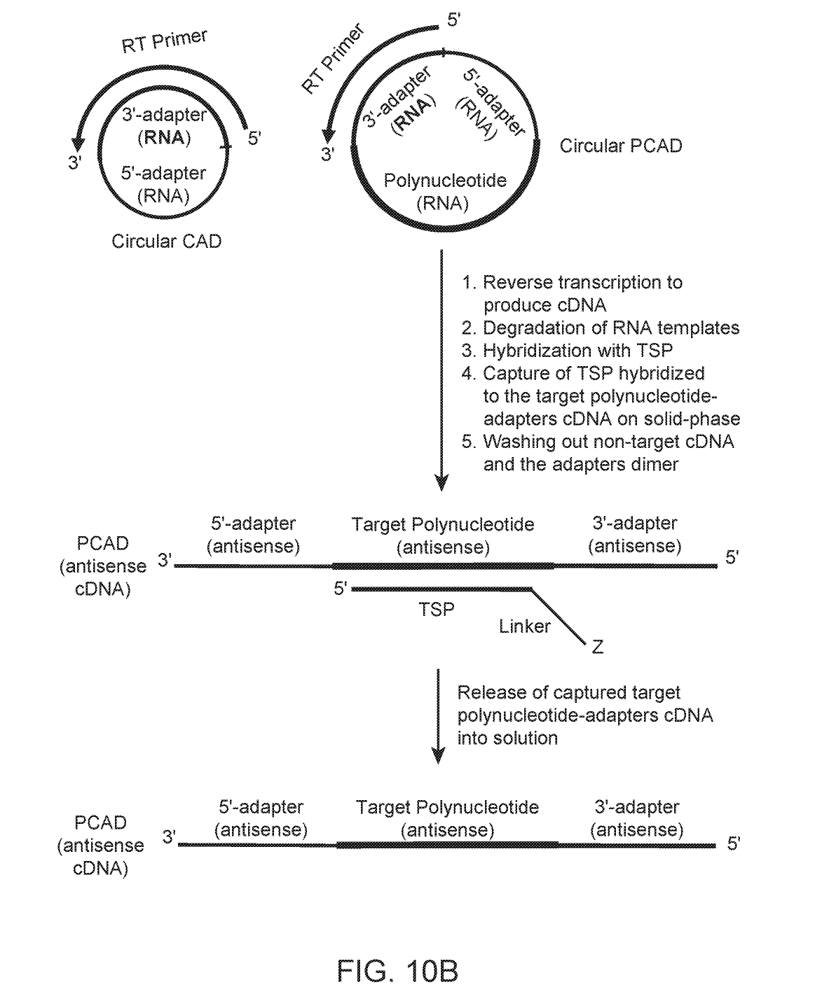

Both polynucleotides and 5′-adapter comprise RNA nucleotides while the 3′-adapter comprises either DNA (FIG. 10A) or RNA nucleotides (FIG. 10B). Unrestricted primer extension on the circular PCAD template can result in synthesis by rolling-circle amplification (RCA) of multimeric cDNAs comprising multiple repeats of the adapter and polynucleotide sequences. Alternatively (as shown in these figures), the PCAD may comprise a CAD with template-deficient modification(s) as described in FIG. 8. In the latter case, primer extension on the circular PCAD template stops at the template-deficient modification(s) after one round, thus preventing RCA. This product of primer extension (cDNA) comprises sequences complementary to the PCAD and contains sequences of a single polynucleotide inserted between the sequencing adapters exactly in the same order as they appear in conventional methods of sequencing library preparation using ligation of two separate adapters to each polynucleotide (see FIG. 5). After reverse transcription and degradation of RNA templates (e.g. by RNase H), the cDNAs comprising antisense sequences of target polynucleotides are captured and separated from cDNA products from non-target polynucleotides and adapter dimers similar to what is shown in FIG. 5. By limiting the method to a single round of primer extension, the methods disclosed herein provide several advantages. One advantage is the generation of standard-length PCR amplicons directly compatible with next generation sequencing (see FIG. 7). Another advantage is reduced sequencing bias for sample polynucleotides varying in sequence and length since these various polynucleotides can be amplified by RCA with different efficiency.

### Example 10. Targeted Sequencing of Selected Nucleic Acids

A pool of sample nucleic acids is purified from at least one source and in some embodiments, ligated to an adapter, such as a CAD on the 3′ or 5′ terminus using a ligase, a buffer, and optionally a ribonuclease inhibitor. In some embodiments, TSPs comprising a hapten and targeting at least one target nucleic acid are prepared, and optionally immobilized on a solid support. After hybridization of the TSPs to the nucleic acids, the target nucleic acids bound to the TSPs are enriched by washing away unbound nucleic acids and unbound adapters, and then released from the TSPs. In some embodiments, TSP hybridization/purification occurs before adapter ligation. In some embodiments, the polynucleotide-adapter constructs are dephosphorylated, exposed to ligase, a buffer, and optionally a ribonuclease inhibitor to generate a circularized product. The target nucleic acids are reverse transcribed to generate a complementary DNA library. The DNA library is amplified to generate sequencing libraries of target nucleic acids, and the libraries are sequenced.

### Example 11. Preparation of Sequencing Library and Targeted Sequencing of Selected miRNAs

Specific miRNAs (Table 4) were purified from 200 μl of a single human plasma sample from a healthy volunteer (Innovative Research) by using a custom lysis solution and subsequent extraction with streptavidin-coupled magnetic beads and biotinylated target specific probes (TSPs) (Table 5). Additionally, RNA was purified from 200 μl of the same single human plasma sample from a healthy volunteer (Innovative Research) using the Zymo Quick-cfRNA Serum & Plasma kit (Catalog No. R1059).

Total RNA or specific miRNAs purified from each plasma sample were used as input for library preparation by ligating them to a Combo Adapter (CAD) (Seq ID NOS 1 and 152). Ligation of the pre-adenylated CAD to the 3′ end of the miRNAs was performed with truncated RNA ligase 2 [Rnl2(tr)] (NEB). The reaction included the RNA samples (total RNAs or specific miRNAs), 1×T4 RNA ligase buffer, 200 units of Rnl2(tr), 40 units of RNase OUT (Life Technologies), 15% PEG 8000, and 75 ng of single-adapter, in a 20 μl reaction volume. The reaction mix was incubated in a thermocycler for 1 hour at 25° C. followed by 10 minutes at 65° C. To inhibit the amplification of unligated single-adapter, a blocking oligo was ligated to the remaining unligated single-adapters after the ligation of miRNAs was completed. A blocking reaction mix was prepared with 20 μl of the adapter-miRNA ligation reaction, 2 μl of a 10-μM mix of blocking oligo and blocking splint, 400 units of T4 DNA ligase, and 1 unit of T4 polynucleotide kinase (NEB) in 1×T4 RNA ligase buffer in a 22-μl total volume. This reaction mix was incubated for 1 hour at 37° C. and 20 minutes at 65° C. To circularize the miRNA-adapter products, 10 units of T4 RNA ligase 1 and 450 μM ATP (sodium salt at pH 7.0 from NEB) were added to the 22 μl reaction mixture from the adapter blocking step for a final reaction volume of 22 μl. This reaction mix was incubated at 37° C. for 1 hour. Reverse transcription of the circular miRNA-adapter templates was performed with SuperScript IV (Invitrogen). The reaction mix included 24 μl from the circularization reaction, 1×SSIV Buffer (Invitrogen), 40 units of RNase OUT (Life Technologies), 1.25 μM RT primer, 5 mM dNTPs, and 200 units of SuperScript IV in a 40 μl total reaction volume. The reaction mix was incubated for 30 min at 50° C. followed by 10 minutes at 80° C. PCR was performed with LongAmp® Hot Start Taq DNA polymerase (NEB). The reaction included 40 μl from the RT reaction, 1×LongAmp® Taq Reaction Buffer (NEB), 3 mM dNTPs, 0.7 μM Forward PCR Primer, 0.7 μM Reverse Index Primer, and 10 units of LongAmp® Hot Start Taq DNA polymerase in a 100 μl reaction volume. The PCR reaction was performed for 17 cycles. PCR included a first step at 94° C. for 30 seconds, and 17 cycles of 94° C. for 15 seconds, 62° C. for 30 seconds, and 70° C. for 15 seconds, with a final step at 70° C. for 5 minutes.

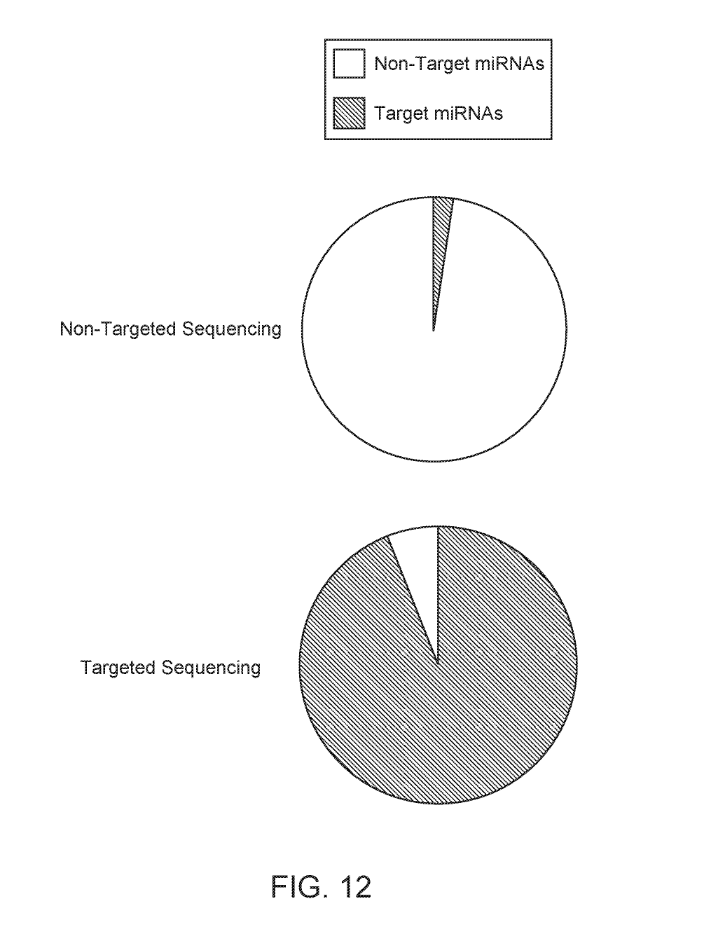

Sequencing libraries were pooled at equimolar concentrations and sequenced in an Illumina MiniSeq instrument with single-end reads of 50 nt following the manufacturer's recommendations. Libraries were mixed with 5% PhiX. The sequencing reads in FastQ files were trimmed of adapter sequences by using Cutadapt [http://dx.doi.org/10.14806/ej.17.1.200)] with the following software parameters: <cutadapt-a TGGAATTCTCGGGTGCCAAGG-m 15> (SEQ ID NO: 2). Trimmed reads were aligned to an index containing all miRNAs on miRBase 21 by using Bowtie2 [Langmead B., Salzberg S. L. (2012) Fast gapped-read alignment with Bowtie 2. Nat. Methods. 9: 357-9]. Analysis of the sequencing results show that for a sample sequenced with a standard non-targeted approach the panel of eight miRNAs represents 2.4% of the miRNA reads detected (FIG. 12, upper panel), while with the targeted approach reads for the panel of eight miRNAs of interest represent 94.1% of the total miRNA reads (FIG. 12, lower panel). This increase in read coverage for a panel of miRNAs of interest allows for better quantification, and for the reduction in the coverage of sequencing required, minimizing in this way the cost per sample.

TABLE 4
List of the selected (target) miRNAs

| | miRNA sequence (5′ to 3′) | |
|---|---|---|
| | (miRNAs are phosphorylated | SEQ |
| miRNA name | at their 5′ end (5′-p) | ID NO. |
| hsa-miR-16-5p | UAGCAGCACGUAAAUAUUGGCG | 25 |
| hsa-miR-17-5p | CAAAGUGCUUACAGUGCAGGUAG | 26 |
| hsa-miR-31-5p | AGGCAAGAUGCUGGCAUAGCU | 46 |
| hsa-miR-125b | UCCCUGAGACCCUAACUUGUGA | 14 |
| hsa-miR-141-3p | UAACACUGUCUGGUAAAGAUGG | 19 |
| hsa-miR-145-5p | GUCCAGUUUUCCCAGGAAUCCCU | 21 |
| hsa-miR-191-5p | CAACGGAAUCCCAAAAGCAGCUG | 30 |
| hsa-miR-524-5p | CUACAAAGGGAAGCACUUUCUC | 62 |

TABLE 5
Target-specific oligonucleotide probes (TSPs)

| | TSP sequence (5′ to 3′) | | |
|---|---|---|---|
| | (Note: All TSPs are | SEQ | Over- |
| | biotinylated at their 3′- | ID | hang |
| TSP name | end via a /3BioTEG/ linker | NO. | design |
| TSP_miR- | CCAATATTTACGTGCTG/3BioTEG/ | 142 | [3 + 2] |
| 16-5p | | | |
| TSP_miR- | ACCTGCACTGTAAGCACT/3BioTEG/ | 143 | [3 + 2] |
| 17-5p | | | |
| TSP_miR- | CTATGCCAGCATCTTG/3BioTEG/ | 144 | [3 + 2] |
| 31-5p | | | |
| TSP_miR- | ACAAGTTAGGGTCTCA/3BioTEG/ | 145 | [4 + 2] |
| 125b | | | |
| TSP_miR- | ATCTTTACCAGACAGT/3BioTEG/ | 146 | [4 + 2] |
| 141-3p | | | |
| TSP_hsa- | GGTTCCTGGGAAAACT/3BioTEG/ | 147 | [4 + 2] |
| miR-145-5p | | | |
| TSP_hsa- | CTGCTTTTGGGATTCC/3BioTEG/ | 148 | [4 + 2] |
| miR-191-5p | | | |
| TSP_hsa- | GAAAGTGCTTCCCTTTG/3BioTEG/ | 149 | [3 + 2] |
| miR-524-5p | | | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for detecting a target polynucleotide amongst a plurality of sample polynucleotides in a sample, comprising:
a) ligating a first adapter to a first end of the target polynucleotide via a splint-independent ligation reaction to produce a single-adapter-polynucleotide ligation product (SAP);
b) either:
i) ligating a second adapter to a second end of the SAP to produce a double-adapter-polynucleotide ligation product (DAP); and optionally hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; or
ii) circularizing the SAP by intramolecular ligation of the SAP ends to produce a circular single adapter-polynucleotide ligation product (CSAP); and optionally hybridizing the primer to the CSAP and extending by the polymerase to produce the primer extension product comprising the sequence complementary to the target polynucleotide; and optionally amplifying the primer extension product to produce the amplified primer extension product comprising sequence(s) corresponding and/or complementary to the target polynucleotide;
c) hybridizing a target-specific oligonucleotide probe (TSP) to at least a portion of the DAP, CSAP, primer extension product, or amplified primer extension product to produce a TSP-hybridized product, and capturing the TSP-hybridized product on a solid support to produce a captured TSP-hybridized product;
d) removing a component from the sample that is not captured on the solid support;
e) releasing the captured TSP-hybridized product into solution to produce a released product; and optionally amplifying the released product to produce an amplified released product; and
f) detecting the released product or amplified released product, wherein the amount of the released product or amplified released product correlates with the amount of the target polynucleotide.

2. The method of claim 1, wherein the target polynucleotide is DNA and the released product or amplified released product comprises a sequence that corresponds and/or is complementary to a sequence of the target polynucleotide.

3. (canceled)

4. The method of claim 1, wherein the target polynucleotide is RNA and the released product or amplified released product comprises a sequence that corresponds and/or is complementary to a sequence of the target polynucleotide.

5. (canceled)

6. (canceled)

7. The method of claim 1, wherein hybridizing the TSP occurs before ligating of the second adapter.

8. (canceled)

9. The method of claim 1, wherein ligating of the second adapter occurs before hybridizing the TSP.

10. (canceled)

11. The method of claim 1, wherein hybridizing the TSP occurs before circularizing.

12. (canceled)

13. The method of claim 1, wherein circularizing occurs before hybridizing the TSP.

14. (canceled)

15. (canceled)

16. The method of claim 1, wherein hybridizing the TSP comprises hybridizing of one TSP oligonucleotide for each product produced in step (a) and/or (b).

17. (canceled)

18. The method of claim 1, comprising ligating the second adapter in step (i) and/or circularizing in step (ii) via a splint-independent reaction.

19. (canceled)

20. (canceled)

21. The method of claim 1, comprising amplifying the released product.

22. The method of claim 1, wherein detecting comprises sequencing the released product.

23. The method of claim 1, wherein detecting comprises performing a microarray detection of the released product.

24-29. (canceled)

30. The method of claim 1, wherein said first adapter is ligated to the 3′ end of the target polynucleotide.

31. The method of claim 1, wherein said adapter comprises a 5′-proximal segment and a 3′-proximal segment, and wherein at least one of the 5′ proximal segment or the 3′ proximal segment comprises a sequencing adapter.

32. The method of claim 1, wherein hybridizing with the TSP occurs in solution followed by capture of the hybridized TSP on a solid support in a later step or steps.

33. (canceled)

34. (canceled)

35. The method of claim 1, wherein said TSP hybridizes to at least a portion of both target polynucleotide and at least a portion of the first or second adapter of the SAP.

36-52. (canceled)

53. The method of claim 1, comprising:
a) ligating a first adapter to a first end of the target polynucleotide via a splint-independent ligation reaction to produce a single-adapter-polynucleotide ligation product (SAP);
b) circularizing the SAP by intramolecular ligation of the SAP ends to produce a circular single adapter-polynucleotide ligation product (CSAP); and hybridizing the primer to the CSAP and extending by the polymerase to produce the primer extension product comprising the sequence complementary to the target polynucleotide;
c) hybridizing a target-specific oligonucleotide probe (TSP) to at least a portion of the primer extension product produced to produce a TSP-hybridized product, and capturing the TSP-hybridized product on a solid support to produce a captured TSP-hybridized product;
d) removing a component from the sample that is not captured on the solid support;
e) releasing the captured TSP-hybridized product into solution to produce a released product; and amplifying the released product to produce an amplified released product; and
f) detecting the amplified released product, wherein the amount of the released product or amplified released product correlates with the amount of the target polynucleotide.

54. The method of claim 1, comprising:
a. ligating a first adapter to a first end of the target polynucleotide via a splint-independent ligation reaction to produce a single-adapter-polynucleotide ligation product (SAP);
b. circularizing the SAP by intramolecular ligation of the SAP ends to produce a circular single adapter-polynucleotide ligation product (CSAP); and
c. hybridizing a target-specific oligonucleotide probe (TSP) to at least a portion of the CSAP to produce a TSP-hybridized product, and capturing the TSP-hybridized product on a solid support to produce a captured TSP-hybridized product;
d. removing a component from the sample that is not captured on the solid support;
e. releasing the captured TSP-hybridized product into solution to produce a released product and amplifying the released product to produce an amplified product wherein the amplifying comprises hybridizing the primer to the released product and extending by the polymerase to produce the primer extension product comprising the sequence complementary to the target polynucleotide; and
f. detecting the amplified product, wherein the amount of the amplified product correlates with the amount of the target polynucleotide.

55. A method for detecting a target polynucleotide amongst a plurality of sample polynucleotides in a sample, comprising:
a) ligating a first adapter to a first end of the target polynucleotide via a splint-independent ligation reaction to produce a single-adapter-polynucleotide ligation product (SAP);
b) hybridizing a target-specific oligonucleotide probe (TSP) to at least a portion of the SAP to produce a TSP-hybridized product, and capturing the TSP-hybridized product on a solid support to produce a captured TSP-hybridized product;
c) removing a component from the sample that is not captured on the solid support;
d) releasing the captured TSP-hybridized product into solution to produce a released product; and optionally amplifying the released product to produce an amplified released product; and
e) either:
i) ligating a second adapter to a second end of the SAP to produce a double-adapter-polynucleotide ligation product (DAP); and optionally hybridizing a primer to the DAP and extending by a polymerase to produce a primer extension product comprising a sequence complementary to the target polynucleotide; and optionally amplifying the primer extension product to produce an amplified primer extension product comprising sequence(s) corresponding and/or complementary to the target polynucleotide; or
ii) circularizing the SAP by intramolecular ligation of the SAP ends to produce a circular single adapter-polynucleotide ligation product (CSAP); and optionally hybridizing the primer to the CSAP and extending by the polymerase to produce the primer extension product comprising the sequence complementary to the target polynucleotide; and optionally amplifying the primer extension product to produce the amplified primer extension product comprising sequence(s) corresponding and/or complementary to the target polynucleotide;
f) detecting the released product or amplified released product, wherein the amount of the released product or amplified released product correlates with the amount of the target polynucleotide.

56. (canceled)

57. A method for detecting a target polynucleotide amongst a plurality of sample polynucleotides in a sample, comprising:
a) ligating a first adapter to a first end of the target polynucleotide via a splint-independent ligation reaction to produce a single-adapter-polynucleotide ligation product (SAP);
b) either:
i) ligating a second adapter to a second end of the SAP to produce a double-adapter-polynucleotide ligation product (DAP); or
ii) circularizing the SAP by intramolecular ligation of the SAP ends to produce a circular single adapter-polynucleotide ligation product (CSAP);
c) hybridizing a target-specific oligonucleotide probe (TSP) to at least a portion of the DAP or CSAP to produce a TSP-hybridized product, and capturing the TSP-hybridized product on a solid support to produce a captured TSP-hybridized product;
d) removing a component from the sample that is not captured on the solid support;
e) releasing the captured TSP-hybridized product into solution to produce a released product; and hybridizing a primer to the released product and extending by the polymerase to produce the primer extension product comprising the sequence complementary to the target polynucleotide; and optionally amplifying the primer extension product to produce an amplified released product; and
f) detecting the primer extension product or amplified released product, wherein the amount of the primer extension product or amplified released product correlates with the amount of the target polynucleotide.


SEQUENCE LISTING (152 sequences)

SEQ ID NO: 1 - DNA, 21 bp, Artificial Sequence
  Description of Artificial Sequence Synthetic oligonucleotide
tggaattctcgggtgccaagg

SEQ ID NO: 2 - DNA, 21 bp, Artificial Sequence
  Description of Artificial Sequence Synthetic oligonucleotide
tggaattctcgggtgccaagg

SEQ ID NO: 3 - RNA, 26 nt, Artificial Sequence
  Description of Artificial Sequence Synthetic oligonucleotide
guucagaguucuacaguccgacgauc

SEQ ID NO: 4 - RNA, 22 nt, Homo sapiens
ugagguaguagguuguauaguu

SEQ ID NO: 5 - RNA, 22 nt, Homo sapiens
aacccguagauccgaacuugug

SEQ ID NO: 6 - RNA, 21 nt, Homo sapiens
uacaguacugugauaacugaa

SEQ ID NO: 7 - RNA, 23 nt, Homo sapiens
agcagcauuguacagggcuauga

SEQ ID NO: 8 - RNA, 22 nt, Homo sapiens
ccgcacuguggguacuugcugc

SEQ ID NO: 9 - RNA, 23 nt, Homo sapiens
agcagcauuguacagggcuauca

SEQ ID NO: 10 - RNA, 23 nt, Homo sapiens
uacccuguagaaccgaauuugug

SEQ ID NO: 11 - RNA, 22 nt, Homo sapiens
uggagugugacaaugguguuug

SEQ ID NO: 12 - RNA, 24 nt, Homo sapiens
ucccugagacccuuuaaccuguga

SEQ ID NO: 13 - RNA, 22 nt, Homo sapiens
ucacaagucaggcucuugggac

SEQ ID NO: 14 - RNA, 22 nt, Homo sapiens
ucccugagacccuaacuuguga

SEQ ID NO: 15 - RNA, 22 nt, Homo sapiens
ucggauccgucugagcuuggcu

SEQ ID NO: 16 - RNA, 22 nt, Homo sapiens
uucauucggcuguccagaugua

SEQ ID NO: 17 - RNA, 22 nt, Homo sapiens
acucggcguggcgucggucgug

SEQ ID NO: 18 - RNA, 21 nt, Homo sapiens
uaccacaggguagaaccacgg

SEQ ID NO: 19 - RNA, 22 nt, Homo sapiens
uaacacugucugguaaagaugg

SEQ ID NO: 20 - RNA, 21 nt, Homo sapiens
ugagaugaagcacuguagcuc

SEQ ID NO: 21 - RNA, 23 nt, Homo sapiens
guccaguuuucccaggaaucccu

SEQ ID NO: 22 - RNA, 22 nt, Homo sapiens
ugagaacugaauuccauggguu

SEQ ID NO: 23 - RNA, 22 nt, Homo sapiens
ucagugcacuacagaacuuugu

SEQ ID NO: 24 - RNA, 21 nt, Homo sapiens
cuagacugaagcuccuugagg

SEQ ID NO: 25 - RNA, 22 nt, Homo sapiens
uagcagcacguaaauauuggcg

SEQ ID NO: 26 - RNA, 23 nt, Homo sapiens
caaagugcuuacagugcagguag

SEQ ID NO: 27 - RNA, 23 nt, Homo sapiens
aacauucaacgcugucggugagu

SEQ ID NO: 28 - RNA, 24 nt, Homo sapiens
uuuggcaaugguagaacucacacu

SEQ ID NO: 29 - RNA, 22 nt, Homo sapiens
caaagaauucuccuuuugggcu

SEQ ID NO: 30 - RNA, 23 nt, Homo sapiens
caacggaaucccaaaagcagcug

SEQ ID NO: 31 - RNA, 21 nt, Homo sapiens
cugaccuaugaauugacagcc

SEQ ID NO: 32 - RNA, 23 nt, Homo sapiens
ugugcaaaucuaugcaaaacuga

SEQ ID NO: 33 - RNA, 22 nt, Homo sapiens
uucccuuugucauccuaugccu

SEQ ID NO: 34 - RNA, 22 nt, Homo sapiens
cugugcgugugacagcggcuga

SEQ ID NO: 35 - RNA, 21 nt, Homo sapiens
caacaccagucgaugggcugu

SEQ ID NO: 36 - RNA, 21 nt, Homo sapiens
augaccuaugaauugacagac

SEQ ID NO: 37 - RNA, 22 nt, Homo sapiens
uagcuuaucagacugauguuga

SEQ ID NO: 38 - RNA, 23 nt, Homo sapiens
agcuacauugucugcuggguuuc

SEQ ID NO: 39 - RNA, 22 nt, Homo sapiens
aagcugccaguugaagaacugu

SEQ ID NO: 40 - RNA, 22 nt, Homo sapiens
uggcucaguucagcaggaacag

SEQ ID NO: 41 - RNA, 22 nt, Homo sapiens
cauugcacuugucucggucuga

SEQ ID NO: 42 - RNA, 22 nt, Homo sapiens
uucaaguaauccaggauaggcu

SEQ ID NO: 43 - RNA, 21 nt, Homo sapiens
uucacaguggcuaaguuccgc

SEQ ID NO: 44 - RNA, 22 nt, Homo sapiens
cacuagauugugagcuccugga

SEQ ID NO: 45 - RNA, 22 nt, Homo sapiens
uguaaacauccucgacuggaag

SEQ ID NO: 46 - RNA, 21 nt, Homo sapiens
aggcaagaugcuggcauagcu

SEQ ID NO: 47 - RNA, 22 nt, Homo sapiens
aaaagcuggguugagagggcga

SEQ ID NO: 48 - RNA, 22 nt, Homo sapiens
gcugacuccuaguccagggcuc

SEQ ID NO: 49 - RNA, 23 nt, Homo sapiens
aggcaguguaguuagcugauugc

SEQ ID NO: 50 - RNA, 22 nt, Homo sapiens
aauugcacgguauccaucugua

SEQ ID NO: 51 - RNA, 22 nt, Homo sapiens
uuuguucguucggcucgcguga

SEQ ID NO: 52 - RNA, 22 nt, Homo sapiens
acuggacuuggagucagaaggc

SEQ ID NO: 53 - RNA, 23 nt, Homo sapiens
agcucggucugaggccccucagu

SEQ ID NO: 54 - RNA, 23 nt, Homo sapiens
ugaggggcagagagcgagacuuu

SEQ ID NO: 55 - RNA, 23 nt, Homo sapiens
aaugacacgaucacucccguuga

SEQ ID NO: 56 - RNA, 22 nt, Homo sapiens
aaaccguuaccauuacugaguu

SEQ ID NO: 57 - RNA, 22 nt, Homo sapiens
aagacgggaggaaagaagggag

SEQ ID NO: 58 - RNA, 21 nt, Homo sapiens
cggggcagcucaguacaggau

SEQ ID NO: 59 - RNA, 22 nt, Homo sapiens
uccuguacugagcugccccgag

SEQ ID NO: 60 - RNA, 22 nt, Homo sapiens
aaugcacccgggcaaggauucu

SEQ ID NO: 61 - RNA, 20 nt, Homo sapiens
cuacaaagggaagcccuuuc

SEQ ID NO: 62 - RNA, 22 nt, Homo sapiens
cuacaaagggaagcacuuucuc

SEQ ID NO: 63 - RNA, 22 nt, Homo sapiens
ugagaccucuggguucugagcu

SEQ ID NO: 64 - RNA, 22 nt, Homo sapiens
uauugcacuugucccggccugu

SEQ ID NO: 65 - RNA, 23 nt, Homo sapiens
caaagugcuguucgugcagguag

SEQ ID NO: 66 - RNA, 22 nt, Homo sapiens
aacccguagauccgaucuugug

SEQ ID NO: 67 - DNA, 17 bp, Artificial Sequence
  Description of Artificial Sequence Synthetic probe
ctatacaacctactacc

SEQ ID NO: 68 - DNA, 15 bp, Artificial Sequence
  Description of Artificial Sequence Synthetic probe
aagttcggatctacg

SEQ ID NO: 69 - DNA, 17 bp, Artificial Sequence
  Description of Artificial Sequence Synthetic probe
cagttatcacagtactg

SEQ ID NO: 70 - DNA, 15 bp, Artificial Sequence
  Description of Artificial Sequence Synthetic probe
atagccctgtacaat

SEQ ID NO: 71 - DNA, 15 bp, Artificial Sequence
  Description of Artificial Sequence Synthetic probe
caagtacccacagtg

SEQ ID NO: 72 - DNA, 15 bp, Artificial Sequence
  Description of Artificial Sequence Synthetic probe
tagccctgtacaatg

SEQ ID NO: 73 - DNA, 16 bp, Artificial Sequence
  Description of Artificial Sequence Synthetic probe
caaattcggttctaca

SEQ ID NO: 74 - DNA, 15 bp, Artificial Sequence
  Description of Artificial Sequence Synthetic probe
aacaccattgtcaca

SEQ ID NO: 75 - DNA, 15 bp, Artificial Sequence
  Description of Artificial Sequence Synthetic probe
acaggttaaagggtc

SEQ ID NO: 76 - DNA, 15 bp, Artificial Sequence
  Description of Artificial Sequence Synthetic probe
caagagcctgacttg

SEQ ID NO: 77 - DNA, 15 bp, Artificial Sequence
  Description of Artificial Sequence Synthetic probe
caagttagggtctca

SEQ ID NO: 78 - DNA, 14 bp, Artificial Sequence
  Description of Artificial Sequence Synthetic probe
aagctcagacggat

SEQ ID NO: 79 - DNA, 13 bp, Artificial Sequence
  Description of Artificial Sequence Synthetic probe
tggacagccgaat

SEQ ID NO: 80 - DNA, 11 bp, Artificial Sequence
  Description of Artificial Sequence Synthetic probe
accgacgccac

SEQ ID NO: 81 - DNA, 15 bp, Artificial Sequence
  Description of Artificial Sequence Synthetic probe
gtggttctaccctgt

SEQ ID NO: 82 - DNA, 16 bp, Artificial Sequence
  Description of Artificial Sequence Synthetic probe
tctttaccagacagtg

SEQ ID NO: 83 - DNA, 15 bp, Artificial Sequence
  Description of Artificial Sequence Synthetic probe
gctacagtgcttcat

SEQ ID NO: 84 - DNA, 13 bp, Artificial Sequence
  Description of Artificial Sequence Synthetic probe
ttcctgggaaaac

SEQ ID NO: 85 - DNA, 15 bp, Artificial Sequence
  Description of Artificial Sequence Synthetic probe
cccatggaattcagt

SEQ ID NO: 86 - DNA, 15 bp, Artificial Sequence
  Description of Artificial Sequence Synthetic probe
agttctgtagtgcac

SEQ ID NO: 87 - DNA, 15 bp, Artificial Sequence
  Description of Artificial Sequence Synthetic probe
aaggagcttcagtct

SEQ ID NO: 88 - DNA, 15 bp, Artificial Sequence
  Description of Artificial Sequence Synthetic probe
caatatttacgtgct

SEQ ID NO: 89 - DNA, 14 bp, Artificial Sequence
  Description of Artificial Sequence Synthetic probe
ctgcactgtaagca

SEQ ID NO: 90 - DNA, 13 bp, Artificial Sequence
  Description of Artificial Sequence Synthetic probe
cgacagcgttgaa

SEQ ID NO: 91 - DNA, 16 bp, Artificial Sequence
  Description of Artificial Sequence Synthetic probe
gtgagttctaccattg

SEQ ID NO: 92 - DNA, 16 bp, Artificial Sequence
  Description of Artificial Sequence Synthetic probe
cccaaaaggagaattc

SEQ ID NO: 93 - DNA, 13 bp, Artificial Sequence
  Description of Artificial Sequence Synthetic probe
gcttttgggattc

SEQ ID NO: 94 - DNA, 17 bp, Artificial Sequence
  Description of Artificial Sequence Synthetic probe
ctgtcaattcataggtc

SEQ ID NO: 95 - DNA, 17 bp, Artificial Sequence
  Description of Artificial Sequence Synthetic probe
agttttgcatagatttg

SEQ ID NO: 96 - DNA, 16 bp, Artificial Sequence
  Description of Artificial Sequence Synthetic probe
gcataggatgacaaag

SEQ ID NO: 97 - DNA, 12 bp, Artificial Sequence
  Description of Artificial Sequence Synthetic probe
cgctgtcacacg

SEQ ID NO: 98 - DNA, 13 bp, Artificial Sequence
  Description of Artificial Sequence Synthetic probe
cccatcgactggt

SEQ ID NO: 99 - DNA, 17 bp, Artificial Sequence
  Description of Artificial Sequence Synthetic probe
ctgtcaattcataggtc

SEQ ID NO: 100 - DNA, 16 bp, Artificial Sequence
  Description of Artificial Sequence Synthetic probe
catcagtctgataagc

SEQ ID NO: 101 - DNA, 14 bp, Artificial Sequence
  Description of Artificial Sequence Synthetic probe
ccagcagacaatgt

SEQ ID NO: 102 - DNA, 15 bp, Artificial Sequence
  Description of Artificial Sequence Synthetic probe
agttcttcaactggc

SEQ ID NO: 103 - DNA, 14 bp, Artificial Sequence
  Description of Artificial Sequence Synthetic probe
tcctgctgaactga

SEQ ID NO: 104 - DNA, 14 bp, Artificial Sequence
  Description of Artificial Sequence Synthetic probe
agaccgagacaagt

SEQ ID NO: 105 - DNA, 16 bp, Artificial Sequence
  Description of Artificial Sequence Synthetic probe
tatcctggattacttg

SEQ ID NO: 106 - DNA, 15 bp, Artificial Sequence
  Description of Artificial Sequence Synthetic probe
gaacttagccactgt

SEQ ID NO: 107 - DNA, 15 bp, Artificial Sequence
  Description of Artificial Sequence Synthetic probe
aggagctcacaatct

SEQ ID NO: 108 - DNA, 15 bp, Artificial Sequence
  Description of Artificial Sequence Synthetic probe
cagtcgaggatgttt

SEQ ID NO: 109 - DNA, 13 bp, Artificial Sequence
  Description of Artificial Sequence Synthetic probe
atgccagcatctt

SEQ ID NO: 110 - DNA, 13 bp, Artificial Sequence
  Description of Artificial Sequence Synthetic probe
cctctcaacccag

SEQ ID NO: 111 - DNA, 14 bp, Artificial Sequence
  Description of Artificial Sequence Synthetic probe
ccctggactaggag

SEQ ID NO: 112 - DNA, 16 bp, Artificial Sequence
  Description of Artificial Sequence Synthetic probe
atcagctaactacact

SEQ ID NO: 113 - DNA, 15 bp, Artificial Sequence
  Description of Artificial Sequence Synthetic probe
cagatggataccgtg

SEQ ID NO: 114 - DNA, 13 bp, Artificial Sequence
  Description of Artificial Sequence Synthetic probe
gagccgaacgaac

SEQ ID NO: 115 - DNA, 15 bp, Artificial Sequence
  Description of Artificial Sequence Synthetic probe
ttctgactccaagtc

SEQ ID NO: 116 - DNA, 12 bp, Artificial Sequence
  Description of Artificial Sequence Synthetic probe
gggcctcagacc

SEQ ID NO: 117 - DNA, 14 bp, Artificial Sequence
  Description of Artificial Sequence Synthetic probe
agtctcgctctctg

SEQ ID NO: 118 - DNA, 13 bp, Artificial Sequence
  Description of Artificial Sequence Synthetic probe
cgggagtgatcgt

SEQ ID NO: 119 - DNA, 15 bp, Artificial Sequence
  Description of Artificial Sequence Synthetic probe
cagtaatggtaacgg

SEQ ID NO: 120 - DNA, 14 bp, Artificial Sequence
  Description of Artificial Sequence Synthetic probe
ttctttcctcccgt

SEQ ID NO: 121 - DNA, 14 bp, Artificial Sequence
  Description of Artificial Sequence Synthetic probe
ctgtactgagctgc

SEQ ID NO: 122 - DNA, 13 bp, Artificial Sequence
  Description of Artificial Sequence Synthetic probe
gggcagctcagta

SEQ ID NO: 123 - DNA, 13 bp, Artificial Sequence
  Description of Artificial Sequence Synthetic probe
aatccttgcccgg

SEQ ID NO: 124 - DNA, 12 bp, Artificial Sequence
  Description of Artificial Sequence Synthetic probe
ggcttccctttg

SEQ ID NO: 125 - DNA, 13 bp, Artificial Sequence
  Description of Artificial Sequence Synthetic probe
aagtgcttccctt

SEQ ID NO: 126 - DNA, 14 bp, Artificial Sequence
  Description of Artificial Sequence Synthetic probe
agaacccagaggtc

SEQ ID NO: 127 - DNA, 13 bp, Artificial Sequence
  Description of Artificial Sequence Synthetic probe
gggacaagtgcaa

SEQ ID NO: 128 - DNA, 13 bp, Artificial Sequence
  Description of Artificial Sequence Synthetic probe
cctgcacgaacag

SEQ ID NO: 129 - DNA, 15 bp, Artificial Sequence
  Description of Artificial Sequence Synthetic probe
aagatcggatctacg

SEQ ID NO: 130 - DNA, 21 bp, Artificial Sequence
  Description of Artificial Sequence Synthetic primer
ccttggcacccgagaattcca

SEQ ID NO: 131 - DNA, 49 bp, Artificial Sequence
  Description of Artificial Sequence Synthetic primer
aatgatacggcgaccaccgagatctacacgttcagagttctacagtccg

SEQ ID NO: 132 - DNA, 63 bp, Artificial Sequence
  Description of Artificial Sequence Synthetic primer
caagcagaagacggcatacgagatgccatggtgactggagttccttggcacccgagaattcca

SEQ ID NO: 133 - DNA, 63 bp, Artificial Sequence
  Description of Artificial Sequence Synthetic primer
caagcagaagacggcatacgagataaaatggtgactggagttccttggcacccgagaattcca

SEQ ID NO: 134 - DNA, 63 bp, Artificial Sequence
  Description of Artificial Sequence Synthetic primer
caagcagaagacggcatacgagattgttgggtgactggagttccttggcacccgagaattcca

SEQ ID NO: 135 - DNA, 63 bp, Artificial Sequence
  Description of Artificial Sequence Synthetic primer
caagcagaagacggcatacgagatattccggtgactggagttccttggcacccgagaattcca

SEQ ID NO: 136 - DNA, 63 bp, Artificial Sequence
  Description of Artificial Sequence Synthetic primer
caagcagaagacggcatacgagatagctaggtgactggagttccttggcacccgagaattcca

SEQ ID NO: 137 - DNA, 63 bp, Artificial Sequence
  Description of Artificial Sequence Synthetic primer
caagcagaagacggcatacgagatgtataggtgactggagttccttggcacccgagaattcca

SEQ ID NO: 138 - DNA, 63 bp, Artificial Sequence
  Description of Artificial Sequence Synthetic primer
caagcagaagacggcatacgagattctgaggtgactggagttccttggcacccgagaattcca

SEQ ID NO: 139 - DNA, 63 bp, Artificial Sequence
  Description of Artificial Sequence Synthetic primer
caagcagaagacggcatacgagatgtcgtcgtgactggagttccttggcacccgagaattcca

SEQ ID NO: 140 - DNA, 63 bp, Artificial Sequence
  Description of Artificial Sequence Synthetic primer
caagcagaagacggcatacgagatcgattagtgactggagttccttggcacccgagaattcca

SEQ ID NO: 141 - DNA, 63 bp, Artificial Sequence
  Description of Artificial Sequence Synthetic primer
caagcagaagacggcatacgagatgctgtagtgactggagttccttggcacccgagaattcca

SEQ ID NO: 142 - DNA, 17 bp, Artificial Sequence
  Description of Artificial Sequence Synthetic probe
ccaatatttacgtgctg

SEQ ID NO: 143 - DNA, 18 bp, Artificial Sequence
  Description of Artificial Sequence Synthetic probe
acctgcactgtaagcact

SEQ ID NO: 144 - DNA, 16 bp, Artificial Sequence
  Description of Artificial Sequence Synthetic probe
ctatgccagcatcttg

SEQ ID NO: 145 - DNA, 16 bp, Artificial Sequence
  Description of Artificial Sequence Synthetic probe
acaagttagggtctca

SEQ ID NO: 146 - DNA, 16 bp, Artificial Sequence
  Description of Artificial Sequence Synthetic probe
atctttaccagacagt

SEQ ID NO: 147 - DNA, 16 bp, Artificial Sequence
  Description of Artificial Sequence Synthetic probe
ggttcctgggaaaact

SEQ ID NO: 148 - DNA, 16 bp, Artificial Sequence
  Description of Artificial Sequence Synthetic probe
ctgcttttgggattcc

SEQ ID NO: 149 - DNA, 17 bp, Artificial Sequence
  Description of Artificial Sequence Synthetic probe
gaaagtgcttccctttg

SEQ ID NO: 150 - DNA, 40 bp, Artificial Sequence
  Description of Artificial Sequence Synthetic oligonucleotide
aaaaaaaaaaaaaaaaaaaaaaaaaaaaaaaaaaaaaaaa

SEQ ID NO: 151 - DNA, 45 bp, Artificial Sequence
  Description of Artificial Sequence Synthetic oligonucleotide
acaacaacaacaacaacaacaacaacaacaacaacaacaacaaca

SEQ ID NO: 152 - RNA, 26 nt, Artificial Sequence
  Description of Artificial Sequence Synthetic oligonucleotide
guucagaguucuacaguccgacgauc